(19) United States
(12) Patent Application Publication
(10) Pub. No.: US 2002/0013458 A1
(43) Pub. Date: Jan. 31, 2002

(54) Enzymatic nucleic acid treatment of disases or conditions related to hepatitis c virus infection

(76) Inventors: Lawrence Blatt, Boulder, CO (US); James A. McSwiggen, Boulder, CO (US); Elisabeth Roberts, Federal Heights, CO (US); Pamela A. Pavo, Lafayette, CO (US); Dennis Macejack, Arvada, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/504,231

(22) Filed: Feb. 15, 2000

Related U.S. Application Data
(63) Continuation-in-part of application No. 09/274,553, filed on Mar. 23, 1999.

(51) Int. Cl. C12Q 1/68; C07H 21/02; C07H 21/04; C12P 19/34

(52) U.S. Cl. 536/24.5; 536/23.1; 536/24.1; 435/6; 435/91.31; 435/325; 435/375; 514/44

(57) ABSTRACT

Enzymatic nucleic acid molecules which modulate the expression and/or replication of hepatitis C virus (HCV).

This patent application is a continuation-in-part of Blatt et al., U.S. Ser. No. 09/274,553, filed Mar. 22, 1999 and Blatt et al., U.S. Ser. No. 09/257,608, filed Feb. 24, 1999, which both claim the benefit of Blatt et al., U.S. Ser. No. 60/100,842, filed Sep. 18, 1998, and McSwiggen et al., U.S. Ser. No. 60/083,217 filed Apr. 27, 1998, all of these earlier applications are entitled “ENZYMATIC NUCLEIC ACID TREATMENT OF DISEASES OR CONDITIONS RELATED TO HEPATITIS C VIRUS INFECTION”. Each of these applications are hereby incorporated by reference herein in their entirety including the drawings.

### BACKGROUND OF THE INVENTION

This invention relates to methods and reagents for the treatment of diseases or conditions relating to the hepatitis C virus (HCV) infection.

The following is a discussion of relevant art, none of which is admitted to be prior art to the present invention.

In 1989, the HCV was determined to be an RNA virus and was identified as the causative agent of most non-A non-B viral Hepatitis (Choo et al., Science. 1989; 244:359-362). Unlike retroviruses such as HIV, HCV does not go though a DNA replication phase and no integrated forms of the viral genome into the host chromosome have been detected (Houghton et al., Hepatology 1991;14:381-388). Rather, replication of the coding (plus) strand is mediated by the production of a replicative (minus) strand leading to the generation of several copies of plus strand HCV RNA. The genome consists of a single, large, open-reading frame that is translated into a polyprotein (Kato et al., FEBS Letters. 1991; 280: 325-328). This polyprotein subsequently undergoes post-translational cleavage, producing several viral proteins (Leinbach et al., Virology. 1994: 204:163-169).

Examination of the 9.5-kilobase genome of HCV has demonstrated that the viral nucleic acid can mutate at a high rate (Smith et al., Mol. Evol. 1997 45:238-246). This rate of mutation has led to the evolution of several distinct genotypes of HCV that share approximately 70% sequence identity (Simmonds et al., J. Gen. Virol 1994; 75:1053-1061). It is important to note that these sequences are evolutionarily quite distant. For example, the genetic identity between humans and primates such as the chimpanzee is approximately 98%. In addition, it has been demonstrated that an HCV infection in an individual patient is composed of several distinct and evolving quasi-species that have 98% identity at the RNA level. Thus, the HCV genome is hypervariable and continuously changing. Although the HCV genome is hypervariable, there are 3 regions of the genome that are highly conserved. These conserved sequences occur in the 5′ and 3′ non-coding regions as well as the 5′-end of the core protein coding region and are thought to be vital for HCV RNA replication as well as translation of the HCV polyprotein. Thus, therapeutic agents that target these conserved HCV genomic regions may have a significant impact over a wide range of HCV genotypes. Moreover, it is unlikely that drug resistance will occur with ribozymes specific to conserved regions of the HCV genome. In contrast, therapeutic modalities that target inhibition of enzymes such as the viral proteases or helicase are likely to result in the selection for drug resistant strains since the RNA for these viral encoded enzymes is located in the hypervariable portion of the HCV genome.

After initial exposure to HCV, the patient will experience a transient rise in liver enzymes, which indicates that inflammatory processes are occurring (Alter et al., IN: Seeff L B, Lewis J H, eds. Current Perspectives in Hepatology. New York: Plenum Medical Book Co; 1989:83-89). This elevation in liver enzymes will occur at least 4 weeks after the initial exposure and may last for up to two months (Farci et al., New England Journal of medicine. 1991:325:98-104). Prior to the rise in liver enzymes, it is possible to detect HCV RNA in the patient's serum using RT-PCR analysis (Takahashi et al., American Journal of Gastroenterology. 1993:88:2:240-243). This stage of the disease is called the acute stage and usually goes undetected since 75% of patients with acute viral hepatitis from HCV infection are asymptomatic. The remaining 25% of these patients develop jaundice or other symptoms of hepatitis.

Acute HCV infection is a benign disease, however, and as many as 80% of acute HCV patients progress to chronic liver disease as evidenced by persistent elevation of serum alanine aminotransferase (ALT) levels and by continual presence of circulating HCV RNA (Sherlock, Lancet 1992; 339:802). The natural progression of chronic HCV infection over a 10 to 20 year period leads to cirrhosis in 20 to 50% of patients (Davis et al., Infectious Agents and Disease 1993;2:150:154) and progression of HCV infection to hepatocellular carcinoma has been well documented (Liang et al., Hepatology. 1993; 18:1326-1333; Tong et al, Western Journal of Medicine, 1994; Vol. 160, No. 2: 133-138). There have been no studies that have determined sub-populations that are most likely to progress to cirrhosis and/or hepatocellular carcinoma, thus all patients have an equal risk of progression.

It is important to note that the survival for patients diagnosed with hepatocellular carcinoma is only 0.9 to 12.8 months from initial diagnosis (Takahashi et aL, American Journal of Gastroenterology. 1993:88:2:240-243). Treatment of hepatocellular carcinoma with chemotherapeutic agents has not proven effective and only 10% of patients will benefit from surgery due to extensive tumor invasion of the liver (Trinchet et al., Presse Medicine. 1994:23:831-833). Given the aggressive nature of primary hepatocellular carcinoma, the only viable treatment alternative to surgery is liver transplantation (Pichlmayr et al., Hepatology. 1994:20:33S-40S).

Upon progression to cirrhosis, patients with chronic HCV infection present with clinical features, which are common to clinical cirrhosis regardless of the initial cause (D'Amico et al., Digestive Diseases and Sciences. 1986;31:5: 468-475). These clinical features may include: bleeding esophageal varices, ascites, jaundice, and encephalopathy (Zakim D, Boyer TD. Hepatology a textbook of liver disease. Second Edition Volume 1. 1990 W. B. Saunders Company. Philadelphia). In the early stages of cirrhosis, patients are classified as compensated meaning that although liver tissue damage has occurred, the patient's liver is still able to detoxify metabolites in the bloodstream. In addition, most patients with compensated liver disease are asymptomatic and the minority with symptoms report only minor symptoms such as dyspepsia and weakness. In the later stages of cirrhosis, patients are classified as decompensated meaning that their ability to detoxify metabolites in the bloodstream is diminished and it is at this stage that the clinical features described above will present.

In 1986, D'Amico et al. described the clinical manifestations and survival rates in 1155 patients with both alcoholic and viral associated cirrhosis (D'Amico supra). Of the 1155 patients, 435 (37%) had compensated disease although 70% were asymptomatic at the beginning of the study. The remaining 720 patients (63%) had decompensated liver disease with 78% presenting with a history of ascites, 31% with jaundice, 17% had bleeding and 16% had encephalopathy. Hepatocellular carcinoma was observed in six (0.5%) patients with compensated disease and in 30 (2.6%) patients with decompensated disease.

Over the course of six years, the patients with compensated cirrhosis developed clinical features of decompensated disease at a rate of 10% per year. In most cases, ascites was the first presentation of decompensation. In addition, hepatocellular carcinoma developed in 59 patients who initially presented with compensated disease by the end of the six-year study.

With respect to survival, the D'Amico study indicated that the five-year survival rate for all patients on the study was only 40%. The six-year survival rate for the patients who initially had compensated cirrhosis was 54% while the six-year survival rate for patients who initially presented with decompensated disease was only 21%. There were no significant differences in the survival rates between the patients who had alcoholic cirrhosis and the patients with viral related cirrhosis. The major causes of death for the patients in the D'Amico study were liver failure in 49%; hepatocellular carcinoma in 22%; and, bleeding in 13% (D'Amico supra).

Chronic Hepatitis C is a slowly progressing inflammatory disease of the liver, mediated by a virus (HCV) that can lead to cirrhosis, liver failure and/or hepatocellular carcinoma over a period of 10 to 20 years. In the US, it is estimated that infection with HCV accounts for 50,000 new cases of acute hepatitis in the United States each year (NIH Consensus Development Conference Statement on Management of Hepatitis C March 1997). The prevalence of HCV in the United States is estimated at 1.8% and the CDC places the number of chronically infected Americans at approximately 4.5 million people. The CDC also estimates that up to 10,000 deaths per year are caused by chronic HCV infection. The prevalence of HCV in the United States is estimated at 1.8% and the CDC places the number of chronically infected Americans at approximately 4.5 million people. The CDC also estimates that up to 10,000 deaths per year are caused by chronic HCV infection.

Numerous well controlled clinical trials using interferon (IFN-alpha) in the treatment of chronic HCV infection have demonstrated that treatment three times a week results in lowering of serum ALT values in approximately 50% (range 40% to 70%) of patients by the end of 6 months of therapy (Davis et al., New England Journal of Medicine 1989; 321:1501-1506; Marcellin et al., Hepatology. 1991; 13:393-397; Tong et al., Hepatology 1997:26:747-754; Tong et al., Hepatology 1997 26(6): 1640-1645). However, following cessation of interferon treatment, approximately 50% of the responding patients relapsed, resulting in a “durable” response rate as assessed by normalization of serum ALT concentrations of approximately 20 to 25%.

In recent years, direct measurement of the HCV RNA has become possible through use of either the branched-DNA or Reverse Transcriptase Polymerase Chain Reaction (RT-PCR) analysis. In general, the RT-PCR methodology is more sensitive and leads to more accurate assessment of the clinical course (Tong et al., supra). Studies that have examined six months of type 1 interferon therapy using changes in HCV RNA values as a clinical endpoint have demonstrated that up to 35% of patients will have a loss of HCV RNA by the end of therapy (Marcellin et al., supra). However, as with the ALT endpoint, about 50% of the patients relapse six months following cessation of therapy resulting in a durable virologic response of only 12% (Marcellin et al., supra). Studies that have examined 48 weeks of therapy have demonstrated that the sustained virological response is up to 25% (NIH consensus statement: 1997). Thus, standard of care for treatment of chronic HCV infection with type 1 interferon is now 48 weeks of therapy using changes in HCV RNA concentrations as the primary assessment of efficacy (Hooftiagle et al., New England Journal of Medicine 1997; 336(5) 347-356).

Side effects resulting from treatment with type 1 interferons can be divided into four general categories, which include 1. Influenza-like symptoms; 2. Neuropsychiatric; 3. Laboratory abnormalities; and, 4. Miscellaneous (Dusheiko et al., Journal of Viral Hepatitis, 1994:1:3-5). Examples of influenza-like symptoms include: fatigue, fever; myalgia; malaise; appetite loss; tachycardia; rigors; headache and arthralgias. The influenza-like symptoms are usually short-lived and tend to abate after the first four weeks of dosing (Dushieko et al., supra). Neuropsychiatric side effects include: irritability, apathy; mood changes; insomnia; cognitive changes and depression. The most important of these neuropsychiatric side effects is depression and patients who have a history of depression should not be given type 1 interferon. Laboratory abnormalities include; reduction in myeloid cells including granulocytes, platelets and to a lesser extent red blood cells. These changes in blood cell counts rarely lead to any significant clinical sequellae (Dushieko et al., supra). In addition, increases in triglyceride concentrations and elevations in serum alanine and aspartate aminotransferase concentration have been observed. Finally, thyroid abnormalities have been reported. These thyroid abnormalities are usually reversible after cessation of interferon therapy and can be controlled with appropriate medication while on therapy. Miscellaneous side effects include nausea; diarrhea; abdominal and back pain; pruritus; alopecia; and rhinorrhea. In general, most side effects will abate after 4 to 8 weeks of therapy (Dushieko et aL, supra).

Welch et al., Gene Therapy 1996 3(11): 994-1001 describe in vitro an in vivo studies with two vector expressed hairpin ribozymes targeted against hepatitis C virus.

Sakamoto et al., J. Clinical Investigation 1996 98(12): 2720-2728 describe intracellular cleavage of hepatitis C virus RNA and inhibition of viral protein translation by certain vector expressed hammerhead ribozymes.

Lieber et al., J. Virology 1996 70(12): 8782-8791 describe elimination of hepatitis C virus RNA in infected human hepatocytes by adenovirus-mediated expression of certain hammerhead ribozymes.

Ohkawa et al., 1997, J. Hepatology, 27; 78-84, describe in vitro cleavage of HCV RNA and inhibition of viral protein translation using certain in vitro transcribed hammerhead ribozymes.

Barber et al., International PCT Publication No. WO 97/32018, describe the use of an adenovirus vector to express certain anti-hepatitis C virus hairpin ribozymes.

Kay et al., International PCT Publication No. WO 96/18419, describe certain recombinant adenovirus vectors to express anti-HCV hammerhead ribozyme.

Yamada et al., Japanese Patent Application No. JP 07231784 describe a specific poly-(L)-lysine conjugated hammerhead ribozyme targeted against HCV.

Draper, U.S. Pat. Nos. 5,610,054 and 5,869,253, describe enzymatic nucleic acid molecules capable of inhibiting replication of HCV.

### SUMMARY OF THE INVENTION

This invention relates to ribozymes, or enzymatic nucleic acid molecules, directed to cleave RNA species of hepatitis C virus (HCV) and/or encoded by the HCV. In particular, applicant describes the selection and function of ribozymes capable of specifically cleaving HCV RNA. Such ribozymes may be used to treat diseases associated with HCV infection.

Due to the high sequence variability of the HCV genome, selection of ribozymes for broad therapeutic applications would likely involve the conserved regions of the HCV genome. Specifically, the present invention describes hammerhead ribozymes that would cleave in the conserved regions of the HCV genome. A list of the thirty hammerhead ribozymes derived from the conserved regions (5′-Non Coding Region (NCR), 5′-end of core protein coding region, and 3′-NCR) of the HCV genome is shown in Table IV. In general, Applicant has found that enzymatic nucleic acid molecules that cleave sites located in the 5′ end of the HCV genome would block translation while ribozymes that cleave sites located in the 3′ end of the genome would block RNA replication. Approximately 50 HCV isolates have been identified and a sequence alignment of these isolates from genotypes 1a, 1b, 2a, 2b, 2c, 3a, 3b, 4a, 5a, and 6 was performed. These alignments were used by the Applicant to identify 30 hammerhead ribozymes sites within regions highly conserved between genotypes. Twenty-three ribozyme sites were identified in regions of greatest homology within the conserved region. Therefore, one ribozyme can be designed to cleave all the different isolates of HCV. According to the Applicant, ribozymes designed against conserved regions of various HCV isolates will enable efficient inhibition of HCV replication in diverse patient populations and may ensure the effectiveness of the ribozymes against HCV quasi species which evolve due to mutations in the non-conserved regions of the HCV genome.

In another preferred embodiment, the invention features the use of an enzymatic nucleic acid molecule, preferably in the hammerhead, NCH motif (Inozyme), G-cleaver, amberzyme, zinzyme and/or DNAzyme motif, to inhibit the expression of HCV RNA.

In yet another preferred embodiment, the invention features the use of an enzymatic nucleic acid molecule, preferably in the hammerhead, Inozyme, G-cleaver, amberzyme, zinzyme and/or DNAzyme motif, to inhibit the expression of HCV minus strand RNA.

By “inhibit” it is meant that the activity of HCV or level of RNAs or equivalent RNAs encoding one or more protein subunits of HCV is reduced below that observed in the absence of the nucleic acid molecules of the invention. In one embodiment, inhibition with enzymatic nucleic acid molecule preferably is below that level observed in the presence of an enzymatically inactive or attenuated molecule that is able to bind to the same site on the target RNA, but is unable to cleave that RNA. In another embodiment, inhibition of HCV genes with the nucleic acid molecule of the instant invention is greater than in the presence of the nucleic acid molecule than in its absence.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% may also be useful in this invention. The nucleic acids may be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not meant to be limiting and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA).

By “nucleic acid molecule” as used herein is meant a molecule having nucleotides. The nucleic acid can be single, double, or multiple stranded and may comprise modified or unmodified nucleotides or non-nucleotides or various mixtures and combinations thereof.

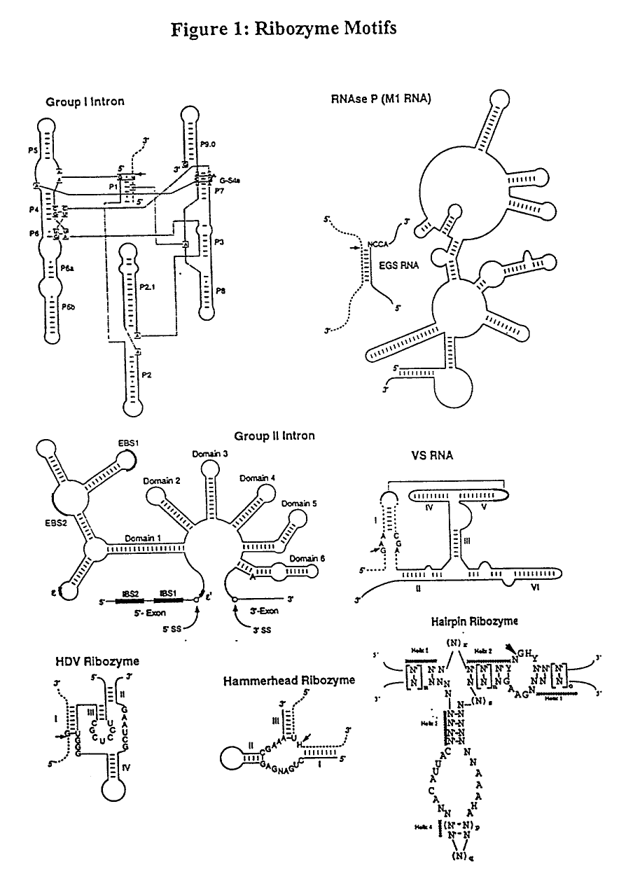

By “enzymatic portion” or “catalytic domain” is meant that portion/region of the ribozyme essential for cleavage of a nucleic acid substrate (for example, see FIG. 1).

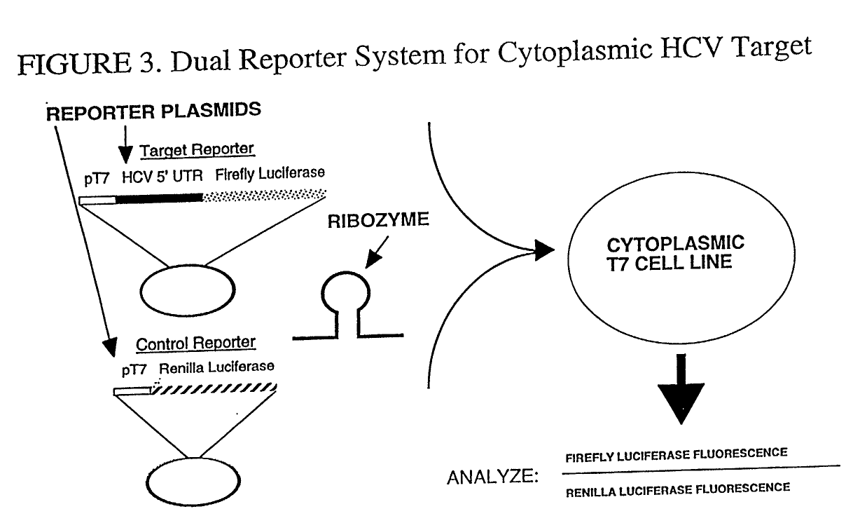

By “substrate binding arm” or “substrate binding domain” is meant that portion/region of a ribozyme which is complementary to (i.e., able to base-pair with) a portion of its substrate. Generally, such complementary is 100%, but can be less if desired. For example, as few as 10 bases out of 14 may be base-paired. Such arms are shown generally in FIGS. 1 and 3. That is, these arms contain sequences within a ribozyme which are intended to bring ribozyme and target RNA together through complementary base-pairing interactions. The ribozyme of the invention may have binding arms that are contiguous or non-contiguous and may be of varying lengths. The length of the binding arm(s) are preferably greater than or equal to four nucleotides; specifically 12-100 nucleotides; more specifically 14-24 nucleotides long. If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “Inozyme” motif is meant, an enzymatic nucleic acid molecule comprising a motif as described in Ludwig et al., U.S. Ser. No. 09/406,643, filed Sep. 27, 1999, entitled “COMPOSITIONS HAVING RNA CLEAVING ACTIVITY”, and International PCT publication Nos. WO 98/58058 and WO 98/58057, all incorporated by reference herein in their entirety including the drawings.

By “G-cleaver” motif is meant, an enzymatic nucleic acid molecule comprising a motif as described in Eckstein et al., International PCT publication No. WO 99/16871, incorporated by reference herein in its entirety including the drawings.

By “zinzyme” motif is meant, a class II enzymatic nucleic acid molecule comprising a motif as described in Beigelman et al., International PCT publication No. WO 99/55857, incorporated by reference herein in its entirety including the drawings. By “amberzyme” motif is meant, a class I enzymatic nucleic acid molecule comprising a motif as described in Beigelman et al., International PCT publication No. WO 99/55857, incorporated by reference herein in its entirety including the drawings.

By ‘DNAzyme’ is meant, an enzymatic nucleic acid molecule that does not require the presence of a 2′-OH group for its activity. In particular embodiments, the enzymatic nucleic acid molecule may have an attached linker(s) or other attached or associated groups, moieties, or chains containing one or more nucleotides with 2′-OH groups.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover.

By “stably interact” is meant, interaction of the oligonucleotides with target nucleic acid (e.g., by forming hydrogen bonds with complementary nucleotides in the target under physiological conditions).

By “equivalent” RNA to HCV is meant to include those naturally occurring RNA molecules associated with HCV infection in various animals, including human, rodent, primate, rabbit and pig. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

In one of the preferred embodiments of the inventions herein, the enzymatic nucleic acid molecule is formed in a hammerhead or hairpin motif, but may also be formed in the motif of a hepatitis d virus, group I intron, group II intron or RNaseP RNA (in association with an RNA guide sequence) or Neurospora VS RNA. Examples of such hammerhead motifs are described by Dreyfus, supra, Rossi et al., 1992, AIDS Research and Human Retroviruses 8, 183; Hairpin motifs are described by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; The hepatitis d virus motif is described by Perrotta and Been, 1992 Biochemistry 31, 16; The RNaseP motif is described by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group II introns are described by Griffin et al., 1995, Chem. Biol. 2, 761; Michels and Pyle, 1995, Biochemistry 34, 2965; Pyle et al., International PCT Publication No. WO 96/22689; The Group I intron is described by Cech et al., U.S. Pat. No. 4,987,071; and the DNAzyme motif is described by Chartrand et al., 1995, Nucleic Acids Research 23, 4092; Santoro et al., 1997, PNAS 94, 4262. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

By “complementarity” is meant that a nucleic acid can form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., ribozyme cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

In a preferred embodiment, the invention provides a method for producing a class of enzymatic cleaving agents which exhibit a high degree of specificity for the RNA of a desired target. The enzymatic nucleic acid molecule is preferably targeted to a highly conserved sequence region of a target mRNAs encoding HCV proteins such that specific treatment of a disease or condition can be provided with either one or several enzymatic nucleic acids. Such enzymatic nucleic acid molecules can be delivered exogenously to specific cells as required. Alternatively, the ribozymes can be expressed from DNA/RNA vectors that are delivered to specific cells.

By “highly conserved sequence region” is meant, a nucleotide sequence of one or more regions in a target gene does not vary significantly from one generation to the other or from one biological system to the other.

Such ribozymes are useful for the prevention of the diseases and conditions discussed above, and any other diseases or conditions that are related to the levels of HCV activity in a cell or tissue.

By “related” is meant that the inhibition of HCV RNAs and thus reduction in the level respective viral activity will relieve to some extent the symptoms of the disease or condition.

In preferred embodiments, the ribozymes have binding arms which are complementary to the target sequences in Tables IV-VIII and X. Examples of such ribozymes are also shown in Tables IV-X. Examples of such ribozymes consist essentially of sequences defined in these tables. Other sequences may be present which do not interfere with such cleavage.

By “consists essentially of” is meant that the active ribozyme contains an enzymatic center or core equivalent to those in the examples, and binding arms able to bind mRNA such that cleavage at the target site occurs. Other sequences may be present which do not interfere with such cleavage. Thus, a core region may, for example, include one or more loop or stem-loop structures, which do not prevent enzymatic activity. “X” in the sequences in Tables V-VIII can be such a loop. A core sequence for a hammerhead ribozyme can be CUGAUGAG X CGAA where X=GCCGUUAGGC or other stem II region known in the art.

Thus, in a first aspect, the invention features ribozymes that inhibit gene expression and/or viral replication. These chemically or enzymatically synthesized RNA molecules contain substrate binding domains that bind to accessible regions of their target mRNAs. The RNA molecules also contain domains that catalyze the cleavage of RNA. The RNA molecules are preferably ribozymes of the hammerhead or hairpin motif. Upon binding, the ribozymes cleave the target mRNAs, preventing translation and protein accumulation. In the absence of the expression of the target gene, HCV gene expression and/or replication is inhibited.

In a preferred embodiment, ribozymes are added directly, or can be complexed with cationic lipids, packaged within liposomes, or otherwise delivered to target cells. The nucleic acid or nucleic acid complexes can be locally administered to relevant tissues ex vivo, or in vivo through injection, infusion pump or stent, with or without their incorporation in biopolymers. In another preferred embodiment, the ribozyme is administered to the site of HCV activity (e.g., hepatocytes) in an appropriate liposomal vehicle.

In another aspect of the invention, ribozymes that cleave target molecules and inhibit HCV activity are expressed from transcription units inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the ribozymes are delivered as described above, and persist in target cells. Alternatively, viral vectors may be used that provide for transient expression of ribozymes. Such vectors might be repeatedly administered as necessary. Once expressed, the ribozymes cleave the target mRNA. Delivery of ribozyme expressing vectors could be systemic, such as by intravenous or intramuscular administration, by administration to target cells ex-planted from the patient followed by reintroduction into the patient, or by any other means that would allow for introduction into the desired target cell (for a review see Couture and Stinchcomb, 1996, TIG., 12, 510). In another aspect of the invention, ribozymes that cleave target molecules and inhibit viral replication are expressed from transcription units inserted into DNA, RNA, or viral vectors. Preferably, the recombinant vectors capable of expressing the ribozymes are locally delivered as described above, and transiently persist in smooth muscle cells. However, other mammalian cell vectors that direct the expression of RNA may be used for this purpose.

By “patient” is meant an organism which is a donor or recipient of explanted cells or the cells themselves. “Patient” also refers to an organism to which enzymatic nucleic acid molecules can be administered. Preferably, a patient is a mammal or mammalian cells. More preferably, a patient is a human or human cells.

As used herein “cell” is used in its usual biological sense, and does not refer to an entire multicellular organism, e.g., specifically does not refer to a human. The cell may be present in an organism which may be a human but is preferably a non-human multicellular organism, e.g., birds, plants and mammals such as cows, sheep, apes, monkeys, swine, dogs, and cats. The cell may be prokaryotic (e.g. bacterial cell) or eukaryotic (e.g., mammalian or plant cell).

By RNA is meant a molecule comprising at least one ribonucleotide residue. By “ribonucleotide” is meant a nucleotide with a hydroxyl group at the 2′ position (eg; 2′-OH) of a β-D-ribo-furanose moiety.

By “vectors” is meant any nucleic acid- and/or viral-based technique used to deliver a desired nucleic acid.

These ribozymes, individually, or in combination or in conjunction with other drugs, can be used to treat diseases or conditions discussed above. For example, to treat a disease or condition associated with HCV levels, the patient may be treated, or other appropriate cells may be treated, as is evident to those skilled in the art.

In a further embodiment, the described molecules can be used in combination with other known treatments to treat conditions or diseases discussed above. For example, the described molecules could be used in combination with one or more known therapeutic agents to treat liver failure, hepatocellular carcinoma, cirrhosis, and/or other disease states associated with HCV infection. Additional known therapeutic agents are those comprising antivirals, interferon, and/or antisense compounds.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### DESCRIPTION OF THE PREFERRED EMBODIMENTS

The drawings will first briefly be described.

Drawings

FIG. 1 shows the secondary structure model for seven different classes of enzymatic nucleic acid molecules. Arrow indicates the site of cleavage. --------- indicate the target sequence. Lines interspersed with dots are meant to indicate tertiary interactions. —is meant to indicate base-paired interaction. Group I Intron: P1-P9.0 represent various stem-loop structures (Cech et al., 1994, Nature Struc. Bio., 1, 273).

RNase P (M1RNA): EGS represents external guide sequence (Forster et al., 1990, Science, 249, 783; Pace et al., 1990, J. Biol. Chem., 265, 3587). Group II Intron: 5′SS means 5′ splice site; 3′SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: : I-IV are meant to indicate four stem-loop structures (Been et al., U.S. Pat. No. 5,625,047). Hammerhead Ribozyme: : I-III are meant to indicate three stem-loop structures; stems I-III can be of any length and may be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 may be covalently linked by one or more bases (i.e., r is 1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q” is ≧2 bases. The connecting loop can also be replaced with a non-nucleotide linker molecule. H refers to bases A, U, or C. Y refers to pyrimidine bases. “_” refers to a covalent bond. (Burke et al., 1996, Nucleic Acids & Mol. Biol., 10, 129; Chowrira et al., U.S. Pat. No. 5,631,359).

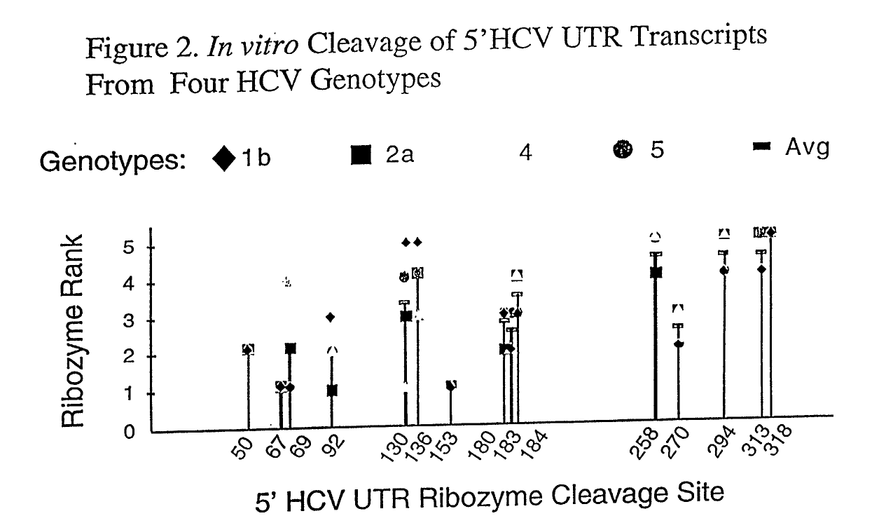

FIG. 2 is a graph displaying the ability of ribozymes targeting various sites within the conserved 5′HCV UTR region to cleave the transcripts made from several genotypes.

FIG. 3 is a schematic representation of the Dual Reporter System utilized to demonstrate ribozyme-mediated reduction of luciferase activity in cell culture.

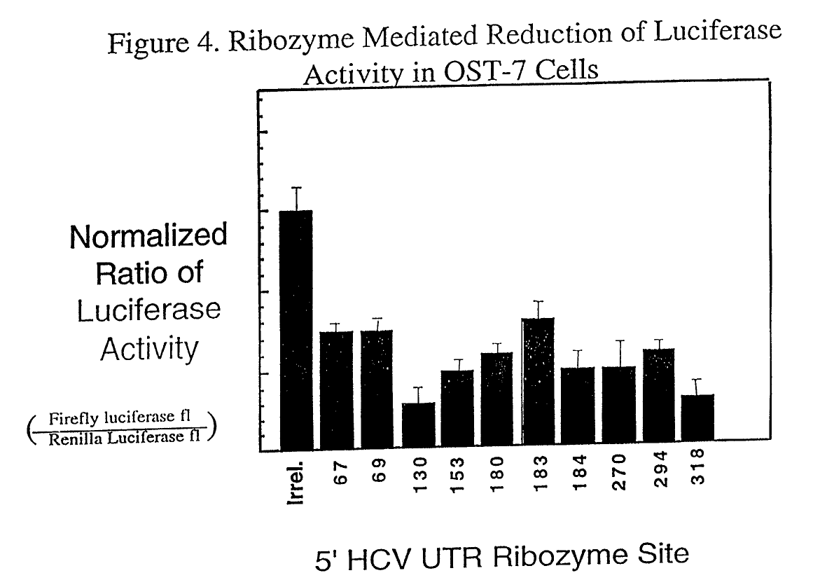

FIG. 4 is a graph demonstrating the ability of ribozymes to reduce luciferase activity in OST-7 cells.

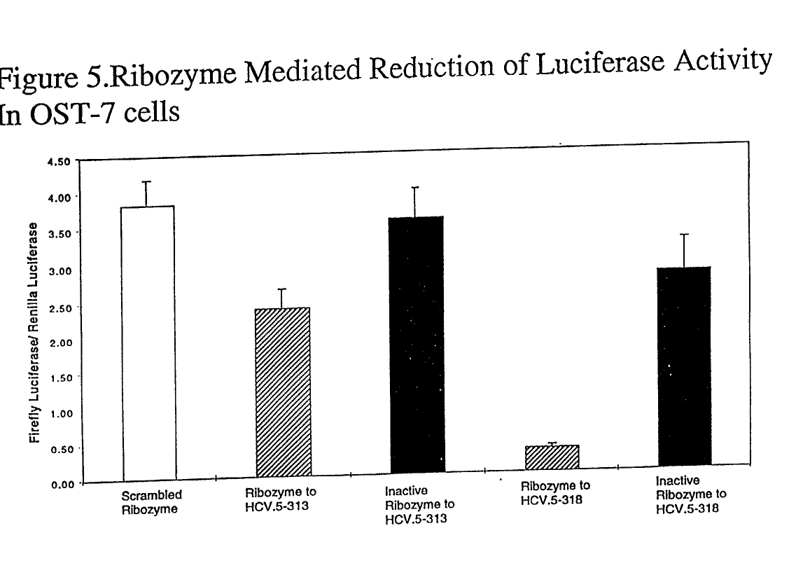

FIG. 5 is a graph demonstrating the ability of ribozymes targeting sites HCV 0.5-313 and HCV 0.5-318, to reduce luciferase activity in OST-7 cells compared to their inactive controls.

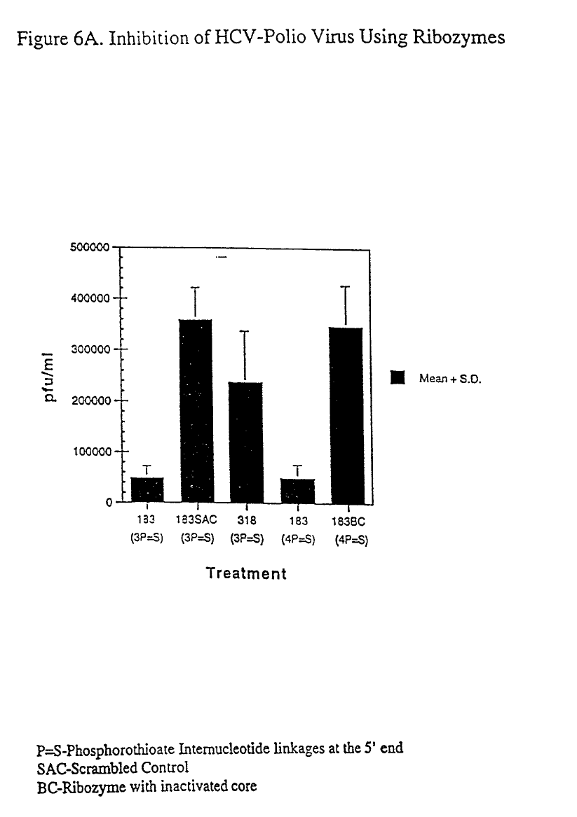

FIG. 6A is a bar graph demonstrating the effect of ribozyme treatment on HCV-Polio virus (PV) replication. HeLa cells in 96-well plates were infected with HCV-PV at a multiplicity of infection (MOI) of 0.1. Virus inoculum was then replaced with media containing 5% serum and ribozyme or control (200nM), as indicated, complexed to a cationic lipid. After 24 hours, cells were lysed 3 times by freeze/thaw and virus was quantified by plaque assay. Scrambled control (SAC), binding control (BAC), 3 P=S ribozymes, and 4 P=S ribozymes are indicated. Plaque forming units (pfu)/ml are shown as the mean of triplicate samples +standard deviation (S.D.).

FIG. 6B is a bar graph demonstrating the effect of ribozyme treatment on wild type PV replication. HeLa cells in 96-well plates were infected with wild type PV at an MOI=0.05 for 30 minutes. All ribozymes contained 4P=S in (B). Plaque forming units (pfu)/ml are shown as the mean of triplicate samples+standard deviation (S.D.).

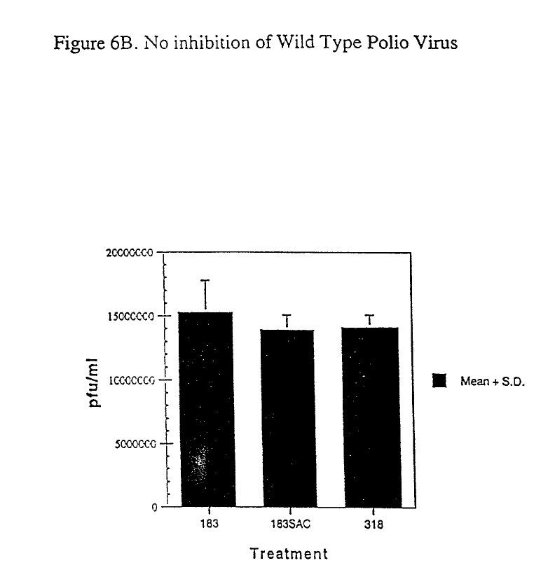

FIG. 7 is a schematic representation of various hammerhead ribozyme constructs targeted against HCV RNA.

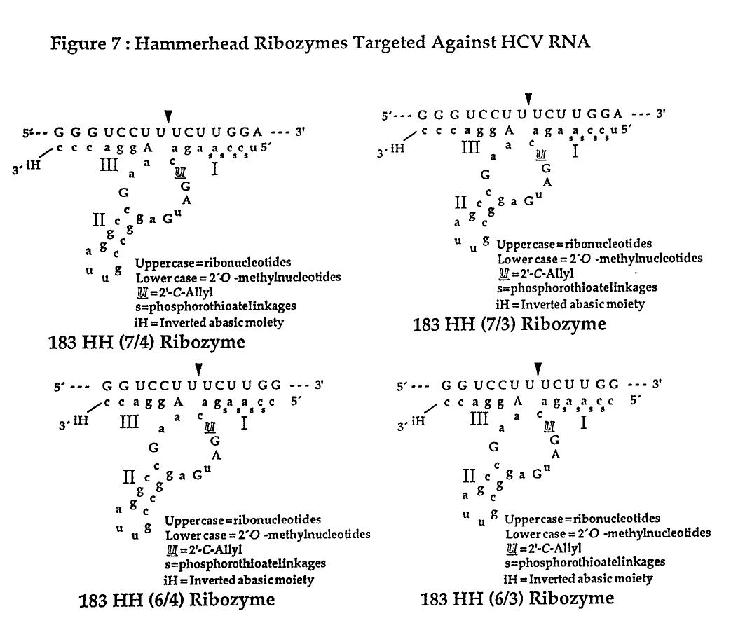

FIG. 8 is a graph demonstrating the effect of site 183 ribozyme treatment on a single round of HCV-PV infection. HeLa cells were infected with HCV-PV at an MOI=5 for 30 minutes prior to treatment with ribozymes or control. Cells were lysed after 6, 7, or 8 hours and virus was quantified by plaque assay. Ribozyme binding arm/stem II formats (7/4, 7/3, 6/4, 6/3) and scrambled control (SAC, 7/4 format) are indicated. All contained 4P=S stabilization. Results in pfu/ml are shown as the median of duplicate samples±range.

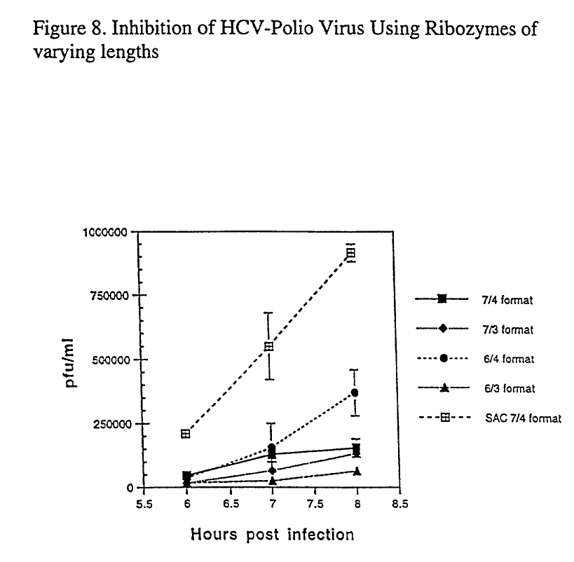

FIG. 9 shows the secondary structure models of three ribozyme motifs described in this application.

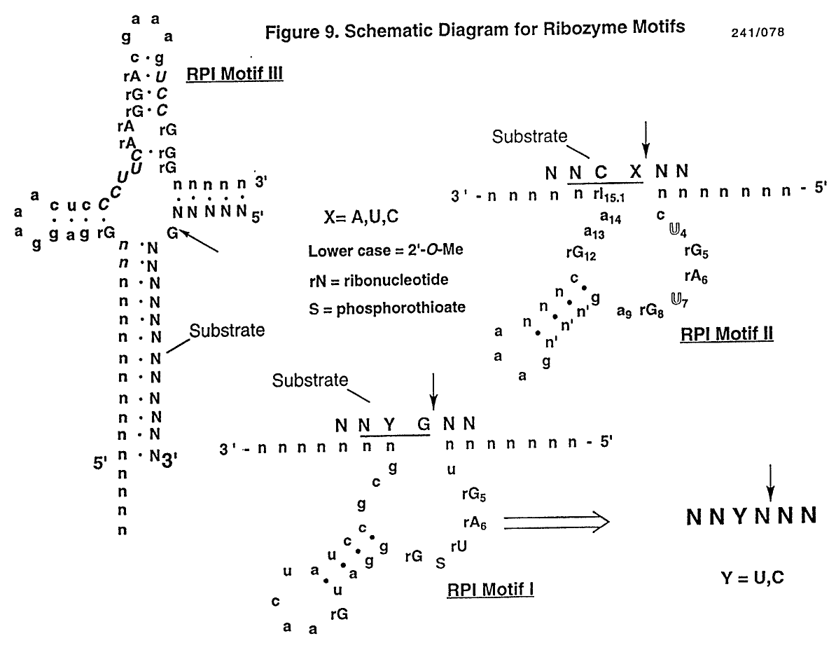

FIG. 10 shows the activity of anti-HCV ribozymes in combination with Interferon. Results in pfu/ml are shown as the median of duplicate samples±range. BAC, binding attenuated control molecule; IF, interferon; Rz, hammerhead ribozyme targeted to HCV site 183; pfu, plaque forming unit.

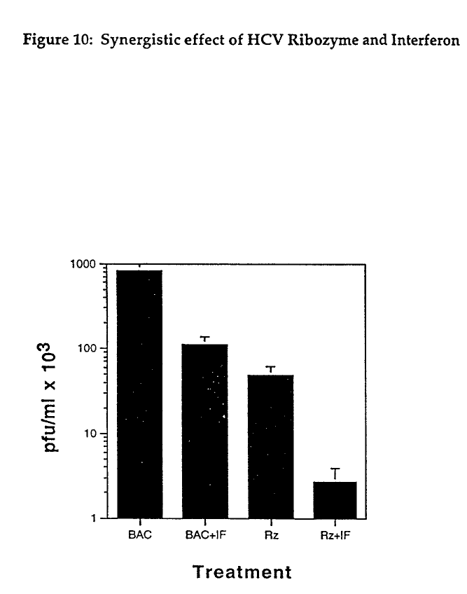

FIG. 11 is a bar graph demonstrating the effect of ribozyme treatment on HCV-Polio virus (PV) replication using anti-HCV ribozymes directed against sites in the HCV minus strand. Both RPI motif I (Hammerhead) and motif II (Inozyme) ribozymes are represented. HeLa cells in 96-well plates were infected with HCV-PV at a multiplicity of infection (MOI) of 0.1. Virus inoculum was then replaced with media containing 5% serum and ribozyme or control (200 nM), as indicated, complexed to a cationic lipid. After 24 hours, cells were lysed 3 times by freeze/thaw and virus was quantified by plaque assay. Scrambled control (SAC) and ribozymes targeting different sites are indicated. Plaque forming units (pfu)/ml are shown as the mean of triplicate samples+standard deviation (S.D.). Ribozymes used in this study are shown in Table X.

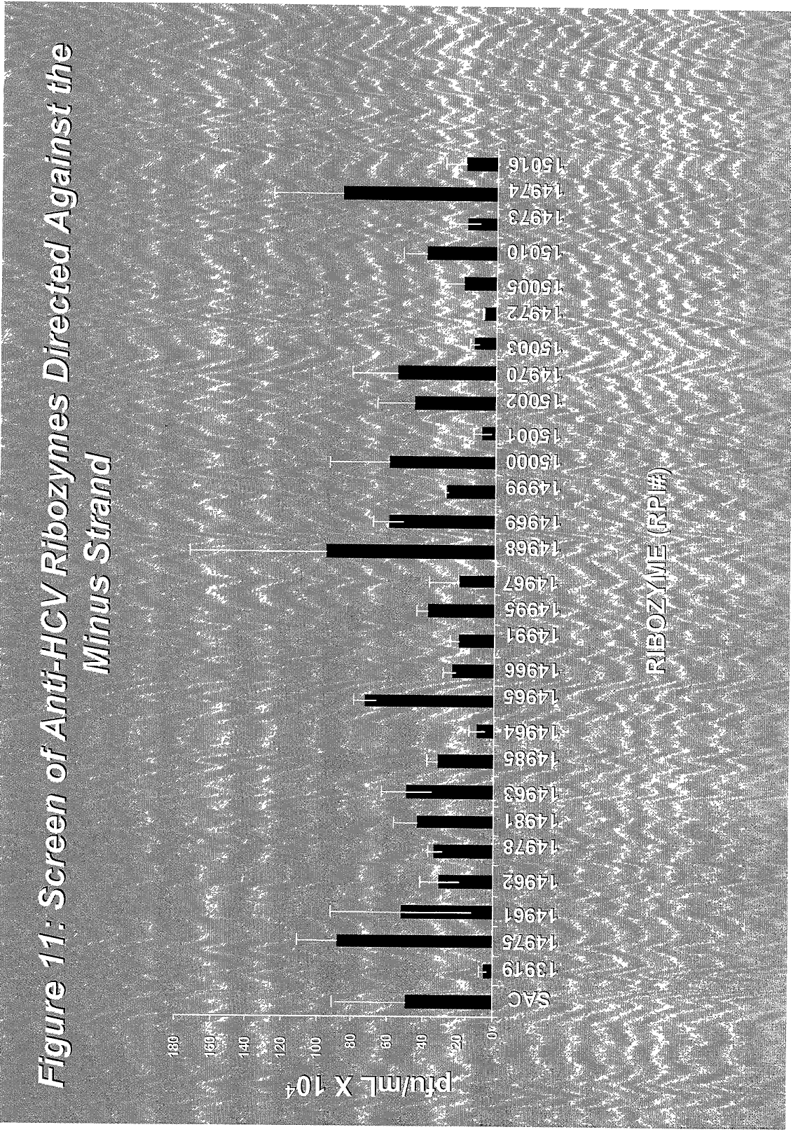

FIG. 12 is a bar graph demonstrating the effect of ribozyme treatment on HCV-Polio virus (PV) replication using anti-HCV ribozymes directed against additional sites in the HCV minus strand. Both RPI motif I and motif II ribozymes are represented. HeLa cells in 96-well plates were infected with HCV-PV at a multiplicity of infection (MOI) of 0.1. Virus inoculum was then replaced with media containing 5% serum and ribozyme or control (200 nM), as indicated, complexed to a cationic lipid. After 24 hours cells, were lysed 3 times by freeze/thaw and virus was quantified by plaque assay. Scrambled control (SAC) and ribozymes targeting different sites are indicated. Plaque forming units (pfu)/ml are shown as the mean of triplicate samples+standard deviation (S.D.). Ribozymes used in this study are shown in Table X.

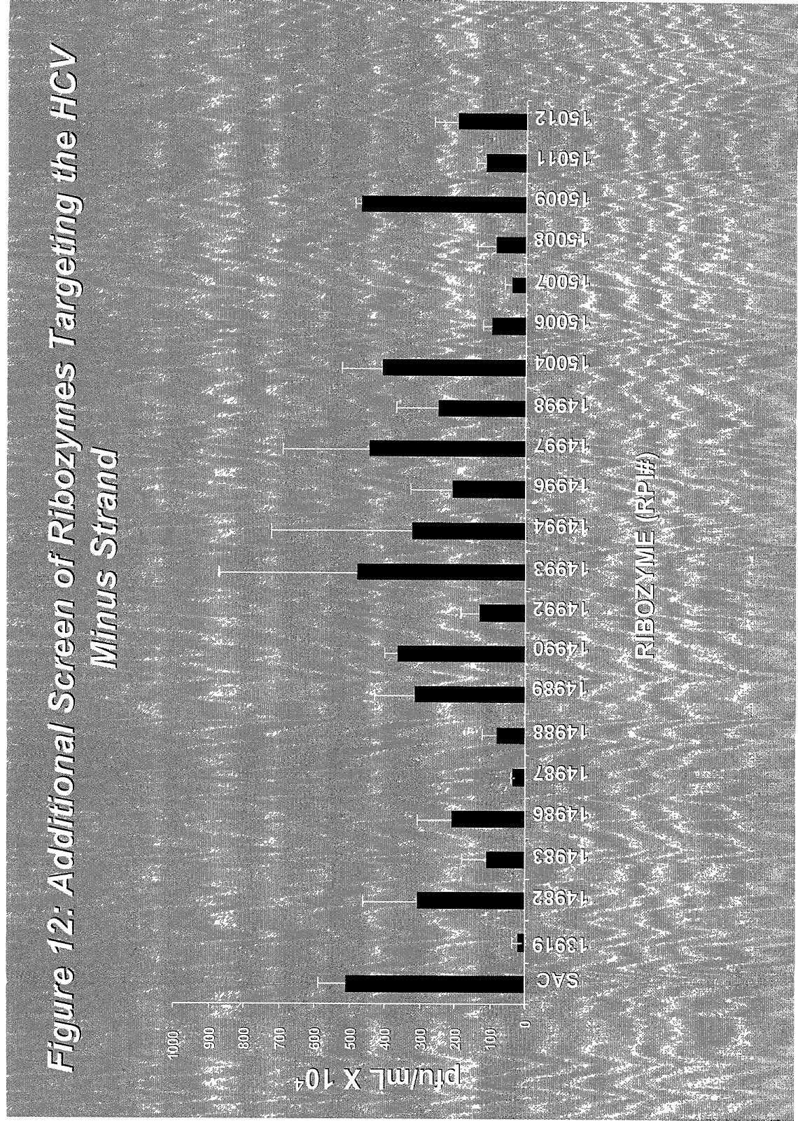

FIG. 13 is a bar graph showing the dose response of a HCV minus strand site 205 directed anti-HCV ribozyme (RPI No. 15006, Table X). Plaque forming units (pfu)/ml are shown as the mean of triplicate samples+standard deviation (S.D.). Results are shown in plaque forming units (pfu)/ml vs. ribozyme concentration in nM.

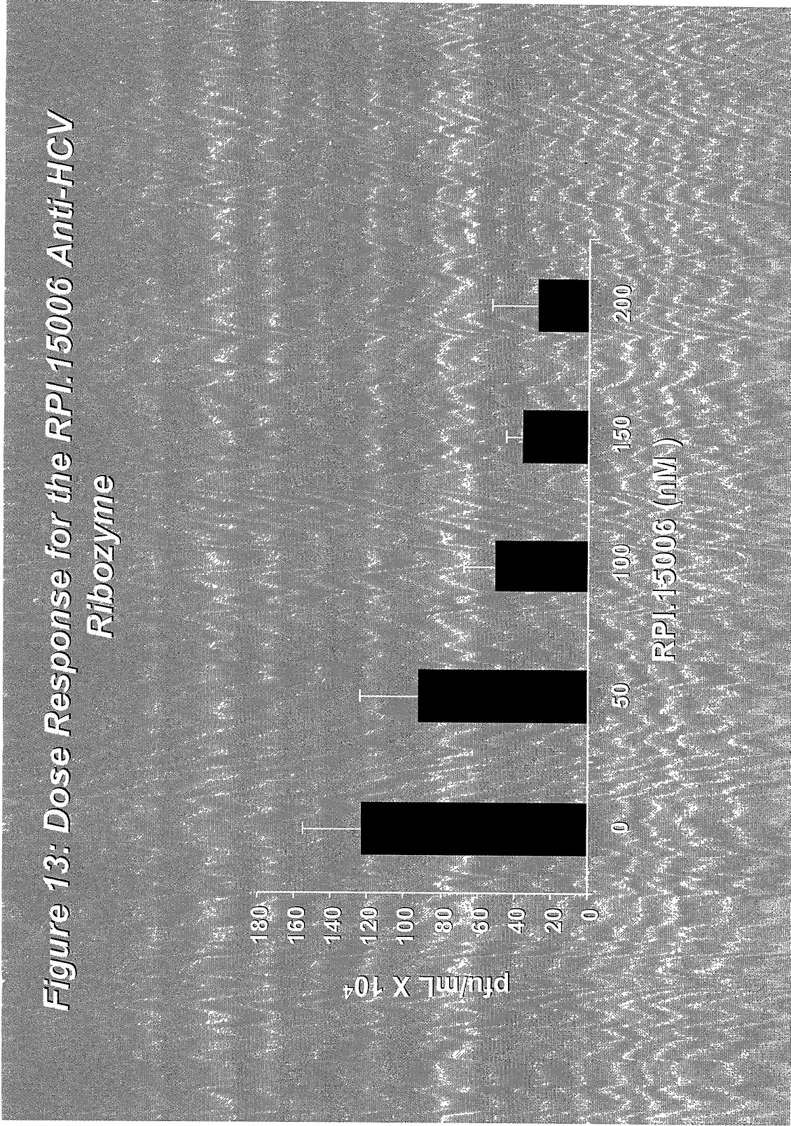

FIG. 14 is a graph showing the dose response of a HCV plus strand site 195 directed anti-HCV ribozyme (RPI No. 13919) when mixed with differing anti-HCV minus strand directed ribozymes (Table X). Results are shown in plaque forming units (pfu)/ml vs. ribozyme concentration in nM.

Ribozymes

Seven basic varieties of naturally-occurring enzymatic RNAs are known presently. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these publications are incorporated by reference herein). Each can catalyze a series of reactions including the hydrolysis of phosphodiester bonds in trans (and thus can cleave other RNA molecules) under physiological conditions. Table I summarizes some of the characteristics of some of these ribozymes. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over other technologies, since the concentration of ribozyme necessary to affect a therapeutic treatment is lower. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a ribozyme.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and efficient cleavage achieved in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Chartrand et al., 1995, Nucleic Acids Research 23, 4092; Santoro et al., 1997, PNAS 94, 4262).

Because of their sequence-specificity, trans-cleaving ribozymes show promise as therapeutic agents for human disease (Usman & McSwiggen, 1995 Ann. Rep. Med. Chem. 30, 285-294; Christoffersen and Marr, 1995 J. Med. Chem. 38, 2023-2037). Ribozymes can be designed to cleave specific RNA targets within the background of cellular RNA. Such a cleavage event renders the RNA non-functional and abrogates protein expression from that RNA. In this manner, synthesis of a protein associated with a disease state can be selectively inhibited.

Ribozymes that cleave the specified sites in HCV RNAs represent a novel therapeutic approach to infection by the hepatitis C virus. Applicant indicates that ribozymes are able to inhibit the activity of HCV and that the catalytic activity of the ribozymes is required for their inhibitory effect. Those of ordinary skill in the art will find that it is clear from the examples described that other ribozymes that cleave HCV RNAs may be readily designed and are within the invention.

Target sites

Targets for useful ribozymes can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468; and, are all hereby incorporated by reference herein in their totalities. Rather than repeat the guidance provided in those documents here, below are provided specific examples of such methods, not limiting to those in the art. Ribozymes to such targets are designed as described in those applications and synthesized to be tested in vitro and in vivo, as also described. Such ribozymes can also be optimized and delivered as described therein.

The sequence of HCV RNAs were screened for optimal ribozyme target sites using a computer folding algorithm. Hammerhead or hairpin ribozyme cleavage sites were identified. These sites are shown in Tables IV-VIII and X (All sequences are 5′ to 3′ in the tables). The nucleotide base position is noted in the tables as that site to be cleaved by the designated type of ribozyme. The nucleotide base position is noted in the tables as that site to be cleaved by the designated type of ribozyme.

Because HCV RNAs are highly homologous in certain regions, some ribozyme target sites are also homologous (see Table IV and VIII). In this case, a single ribozyme will target different classes of HCV RNA. The advantage of one ribozyme that targets several classes of HCV RNA is clear, especially in cases where one or more of these RNAs may contribute to the disease state.

Hammerhead or hairpin ribozymes were designed that could bind and were individually analyzed by computer folding (Jaeger et al., 1989 Proc. Natl. Acad. Sci. USA, 86, 7706) to assess whether the ribozyme sequences fold into the appropriate secondary structure. Those ribozymes with unfavorable intramolecular interactions between the binding arms and the catalytic core are eliminated from consideration. Varying binding arm lengths can be chosen to optimize activity. Generally, at least 5 bases on each arm are able to bind to, or otherwise interact with, the target RNA. Ribozymes of the hammerhead or hairpin motif were designed to anneal to various sites in the mRNA message. The binding arms are complementary to the target site sequences described above.

Ribozyme Synthesis

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., antisense oligonucleotides, hammerhead or the Inozyme ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C. the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA·3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA·3HF (0.1 mL) is added and the vial is heated at 65° C. for 15 min. The sample is cooled at −20° C. and then quenched with 1.5 M NH₄HCO₃.

For anion exchange desalting of the deprotected oligomer, the TEAB solution was loaded onto a Qiagen 500® anion exchange cartridge (Qiagen Inc.) that was prewashed with 50 mM TEAB (10 mL). After washing the loaded cartridge with 50 mM TEAB (10 mL), the RNA was eluted with 2 M TEAB (10 mL) and dried down to a white powder.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes were synthesized by substituting switching the order of G₅A₆ and substituting a U for A,₁₄ (numbering from Hertel, K. J., et al., 1992, Nucleic Acids Res., 20, 3252). Inactive ribozymes were also by synthesized by substituting a U for G5 and a U for A14. In some cases, the sequence of the substrate binding arms were randomized while the overall base composition was maintained.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format, all that is important is the ratio of chemicals used in the reaction.

Hairpin ribozymes are synthesized in two parts and annealed to reconstruct the active ribozyme (Chowrira and Burke, 1992 Nucleic Acids Res., 20, 2835-2840). Ribozymes are also synthesized from DNA templates using bacteriophage T7 RNA polymerase (Milligan and Uhlenbeck, 1989, Methods Enzymol. 180, 51).

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, 30 TIBS 17, 34; Usman et al., 1994, Nucleic Acids Symp. Ser. 31, 163). Ribozymes are purified by gel electrophoresis using general methods or are purified by high pressure liquid chromatography (HPLC; see Wincott et al., supra, the totality of which is hereby incorporated herein by reference) and are re-suspended in water.

The sequences of the ribozymes that are chemically synthesized, useful in this study, are shown in Tables IV to X. Those in the art will recognize that these sequences are representative only of many more such sequences where the enzymatic portion of the ribozyme (all but the binding arms) is altered to affect activity. The ribozyme sequences listed in Tables IV to X may be formed of ribonucleotides or other nucleotides or non-nucleotides. Such ribozymes with enzymatic activity are equivalent to the ribozymes described specifically in the tables.

Optimizing Activity of the nucleic acid molecule of the invention.

Chemically synthesizing nucleic acid molecules with modifications (base, sugar and/or phosphate) that prevent their degradation by serum ribonucleases may increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). All these publications are hereby incorporated by reference herein. Modifications which enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review, see Usman and Cedergren, 1992, TIBS. 17, 34; Usman et al., 1994, Nucleic Acids Symp. Ser. 31, 163; Burgin et al., 1996, Biochemistry, 35, 14090). All of these publications are incorporated by reference herein. Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al, U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications may cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in a cell and/or in vivo the activity may not be significantly lowered. Therapeutic nucleic acid molecules delivered exogenously must optimally be stable within cells until translation of the target RNA has been inhibited long enough to reduce the levels of the undesirable protein. This period of time varies between hours to days depending upon the disease state. Clearly, nucleic acid molecules must be resistant to nucleases in order to function as effective intracellular therapeutic agents. Improvements in the chemical synthesis of RNA and DNA (Wincott et aL, 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Use of these the nucleic acid-based molecules of the invention will lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules may also include combinations of different types of nucleic acid molecules.

Therapeutic nucleic acid molecules (e.g., enzymatic nucleic acid molecules) delivered exogenously must optimally be stable within cells until translation of the target RNA has been inhibited long enough to reduce the levels of the undesirable protein. This period of time varies between hours to days depending upon the disease state. Clearly, these nucleic acid molecules must be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

By “enhanced enzymatic activity” is meant to include activity measured in cells and/or in vivo where the activity is a reflection of both catalytic activity and ribozyme stability. In this invention, the product of these properties is increased or not significantly (less that 10 fold) decreased in vivo compared to an all RNA ribozyme or all DNA enzyme.

In yet another preferred embodiment, nucleic acid catalysts having chemical modifications which maintain or enhance enzymatic activity is provided. Such nucleic acid is also generally more resistant to nucleases than unmodified nucleic acid. Thus, in a cell and/or in vivo the activity may not be significantly lowered. As exemplified herein such ribozymes are useful in a cell and/or in vivo even if activity over all is reduced 10-fold (Burgin et al., 1996, Biochemistry, 35, 14090). Such ribozymes herein are said to “maintain” the enzymatic activity of an all RNA ribozyme.

In another aspect the nucleic acid molecules comprise a 5′and/or a 3′-cap structure.

By “cap structure” is meant chemical modifications, which have been incorporated at either terminus of the oligonucleotide (see, for example, Wincott et al., WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminus (3′-cap) or may be present on both termini. In non-limiting examples: the 5′-cap is selected from the group comprising inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In yet another preferred embodiment, the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details, see Beaucage and Iyer, 1993, Tetrahedron 49, 1925; incorporated by reference herein). By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group may be substituted or unsubstituted. When substituted the substituted group(s) is preferably, hydroxyl, cyano, alkoxy, ═O, ═S, NO₂ or N(CH₃)_{2,} amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group may be substituted or unsubstituted. When substituted the substituted group(s) is preferably, hydroxyl, cyano, alkoxy, ═O, ═S, NO_{2,} halogen, N(CH₃)_{2,} amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO₂ or N(CH₃)₂, amino or SH.

Such alkyl groups may also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra. All these publications are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). All these publications are incorporated by reference herein. By “modified bases” in this aspect is meant nucleotide bases other than adenine, guanine, cytosine and uracil at 1′ position or their equivalents; such bases may be used at any position, for example, within the catalytic core of an enzymatic nucleic acid molecule and/or in the substrate-binding regions of the nucleic acid molecule.

In a preferred embodiment, the invention features modified ribozymes with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, (for more details, see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which may be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Use of these molecules will lead to better treatment of the disease progression by affording the possibility of combination therapies (e.g., multiple ribozymes targeted to different genes, ribozymes coupled with known small molecule inhibitors, or intermittent treatment with combinations of ribozymes (including different ribozyme motifs) and/or other chemical or biological molecules). The treatment of patients with nucleic acid molecules may also include combinations of different types of nucleic acid molecules. Therapies may be devised which include a mixture of ribozymes (including different ribozyme motifs), antisense and/or 2-5A chimera molecules to one or more targets to alleviate symptoms of a disease.

Administration of Ribozymes

Sullivan et al., PCT WO 94/02595, describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination is locally delivered by direct injection or by use of a catheter, infusion pump, stent or other delivery devices such as Alzet® pumps, Medipad® devices. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Sullivan et al., supra and Draper et al., PCT WO93/23569, which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a lipid or liposome delivery mechanism, standard protocols for formulation can be followed. The compositions of the present invention may also be formulated and used as tablets, capsules or elixirs for oral administration; suppositories for rectal administration; sterile solutions; suspensions for injectable administration; and the like.

The present invention also includes pharmaceutically acceptable formulations of the compounds described. These formulations include salts of the above compounds, e.g., acid addition salts, including salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation to reach a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as the HCV infected liver cells.

The invention also features the use of a composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). All these publications are incorporated by reference herein. Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). All these references are incorporated by reference herein. The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of these are incorporated by reference herein).

In addition other cationic molecules may also be utilized to deliver the molecules of the present invention. For example, ribozymes may be conjugated to glycosylated poly(L-lysine) which has been shown to enhance localization of antisense oligonucleotides into the liver (Nakazono et al., 1996, Hepatology 23, 1297-1303; Nahato et al., 1997, Biochem Pharm. 53, 887-895). Glycosylated poly (L-lysine) may be covalently attached to the enzymatic nucleic acid or be bound to enzymatic nucleic acid through electrostatic interaction.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. Id. at 1449. These include sodium benzoate, sorbic acid and esters of p-hydroxybenzoic acid. In addition, antioxidants and suspending agents may be used.

A pharmaceutically effective dose is that dose required to prevent, inhibit the occurrence, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention may also be administered to a patient in combination with other therapeutic compounds to increase the overall therapeutic effect. The use of multiple compounds to treat an indication may increase the beneficial effects while reducing the presence of side effects.

Alternatively, the enzymatic nucleic acid molecules of the instant invention can be expressed within cells from eukaryotic promoters (e.g., Izant and Weintraub, 1985 Science 229, 345; McGarry and Lindquist, 1986 Proc. Natl. Acad. Sci. USA 83, 399; Scanlon et al., 1991, Proc. Natl. Acad. Sci. USA, 88, 10591-5; Kashani-Sabet et al., 1992 Antisense Res. Dev., 2, 3-15; Dropulic et al., 1992 J. Virol, 66, 1432-41; Weerasinghe et al., 1991 J. Virol, 65, 5531-4; Ojwang et al., 1992 Proc. Natl. Acad. Sci. USA 89, 10802-6; Chen et al, 1992 Nucleic Acids Res., 20, 4581-9; Sarver et al., 1990 Science 247, 1222-1225; Thompson et al., 1995 Nucleic Acids Res. 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992 Nucleic Acids Symp. Ser., 27, 15-6; Taira et al., 1991, Nucleic Acids Res., 19, 5125-30; Ventura et al., 1993 Nucleic Acids Res., 21, 3249-55; Chowrira et al., 1994 J. Biol. Chem. 269, 25856; all of these references are hereby incorporated in their totalities by reference herein).

In another aspect of the invention, enzymatic nucleic acid molecules that cleave target molecules are expressed from transcription units (see for example Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the ribozymes are delivered as described above, and persist in target cells. Alternatively, viral vectors may be used that provide for transient expression of ribozymes. Such vectors might be repeatedly administered as necessary. Once expressed, the ribozymes cleave the target mRNA. The active ribozyme contains an enzymatic center or core equivalent to those in the examples, and binding arms able to bind target nucleic acid molecules such that cleavage at the target site occurs. Other sequences may be present which do not interfere with such cleavage. Delivery of ribozyme expressing vectors could be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect the invention features, an expression vector comprising nucleic acid sequence encoding at least one of the nucleic acid catalyst of the instant invention is disclosed. The nucleic acid sequence encoding the nucleic acid catalyst of the instant invention is operable linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector may optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res.., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al, 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al, 1994, Nucleic Acid Res., 22, 2830; Noonberg et al., U.S. Pat. No. 5,624,803; Good et al, 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as, adenovirus or adeno-associated virus vectors), or viral RNA vectors (such as retroviral or alphavirus vectors) (for a review, see Couture and Stinchcomb, 1996, supra).

In yet another aspect, the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule. In another preferred embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment, the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

Interferons

Type I interferons (IFN) are a class of natural cytokines that includes a family of greater than 25 IFN-α (Pesta, 1986, Methods Enzymol. 119, 3-14) as well as IFN-β, and IFN-ω. Although evolutionarily derived from the same gene (Diaz et al., 1994, Genomics 22, 540-552), there are many differences in the primary sequence of these molecules, implying an evolutionary divergence in biologic activity. All type I IFN share a common pattern of biologic effects that begin with binding of the IFN to the cell surface receptor (Pfeffer & Strulovici, 1992, Transmembrane secondary messengers for IFN-α/β. In: Interferon. Principles and Medical Applications., S. Baron, D. H. Coopenhaver, F. Dianzani, W. R. Fleischmann Jr., T. K. Hughes Jr., G. R. Kimpel, D. W. Niesel, G. J. Stanton, and S. K. Tyring, eds. 151-160). Binding is followed by activation of tyrosine kinases, including the Janus tyrosine kinases and the STAT proteins, which leads to the production of several IFN-stimulated gene products (Johnson et al., 1994, Sci. Am. 270, 68-75). The IFN-stimulated gene products are responsible for the pleotropic biologic effects of type I IFN, including antiviral, antiproliferative, and immunomodulatory effects, cytokine induction, and HLA class I and class II regulation (Pestka et al., 1987, Annu. Rev. Biochem 56, 727). Examples of IFN-stimulated gene products include 2-5-oligoadenylate synthetase (2-5 OAS), β₂-microglobulin, neopterin, p68 kinases, and the Mx protein (Chebath & Revel, 1992, The 2-5 A system: 2-5 A synthetase, isospecies and functions. In: Interferon. Principles and Medical Applications. S. Baron, D. H. Coopenhaver, F. Dianzani, W. R. Jr. Fleischmann, T. K. Jr Hughes, G. R. Kimpel, D. W. Niesel, G. J. Stanton, and S. K. Tyring, eds., pp. 225-236; Samuel, 1992, The RNA-dependent P1/eIF-2α protein kinase. In: Interferon. Principles and Medical Applications. S. Baron, D. H. Coopenhaver, F. Dianzani, W. R. Fleischmann Jr., T. K. Hughes Jr., G. R. Kimpel, D. W. Niesel, G. H. Stanton, and S. K. Tyring, eds. 237-250; Horisberger, 1992, MX protein: function and Mechanism of Action. In: Interferon. Principles and Medical Applications. S. Baron, D. H. Coopenhaver, F. Dianzani, W. R. Fleischmann Jr., T. K. Hughes Jr., G. R. Kimpel, D. W. Niesel, G. H. Stanton, and S. K. Tyring, eds. 215-224). Although all type I IFN have similar biologic effects, not all the activities are shared by each type I IFN, and, in many cases, the extent of activity varies quite substantially for each IFN subtype (Fish et al, 1989, J. Interferon Res. 9, 97-114; Ozes et al., 1992, J. Interferon Res. 12, 55-59). More specifically, investigations into the properties of different subtypes of IFN-α, and molecular hybrids of IFN-α. have shown differences in pharmacological properties (Rubinstein, 1987, J. Interferon Res. 7, 545-551). These pharmacological differences may arise from as few as three amino acid residue changes (Lee et al., 1982, Cancer Res. 42, 1312-1316).

Eighty-five to 166 amino acids are conserved in the known IFN-α subtypes. Excluding the IFN-α pseudogenes, there are approximately 25 known distinct IFN-α subtypes. Pairwise comparisons of these nonallelic subtypes show primary sequence differences ranging from 2% to 23%. In addition to the naturally occurring IFNs, a non-natural recombinant type I interferon known as consensus interferon (CIFN) has been synthesized as a therapeutic compound (Tong et al., 1997, Hepatology 26, 747-754).

Interferon is currently in use for at least 12 different indications including infectious and autoimmune diseases and cancer (Borden, 1992, N. Engl. J. Med. 326, 1491-1492). For autoimmune diseases IFN has been utilized for treatment of rheumatoid arthritis, multiple sclerosis, and Crohn's disease. For treatment of cancer IFN has been used alone or in combination with a number of different compounds. Specific types of cancers for which IFN has been used include squamous cell carcinomas, melanomas, hypemephromas, hemangiomas, hairy cell leukemia, and Kaposi's sarcoma. In the treatment of infectious diseases, IFNs increase the phagocytic activity of macrophages and cytotoxicity of lymphocytes and inhibits the propagation of cellular pathogens. Specific indications for which IFN has been used as treatment include: hepatitis B, human papillomavirus types 6 and 11 (i.e. genital warts) (Leventhal et al., 1991, N Engl J Med 325, 613-617), chronic granulomatous disease, and hepatitis C virus.

Numerous well controlled clinical trials using IFN-alpha in the treatment of chronic HCV infection have demonstrated that treatment three times a week results in lowering of serum ALT values in approximately 50% (range 40% to 70%) of patients by the end of 6 months of therapy (Davis et al., 1989, New England Journal of Medicine 321, 1501-1506; Marcellin et al., 1991, Hepatology 13, 393-397; Tong et al., 1997, Hepatology 26, 747-754; Tong et al., Hepatology 26, 1640-1645). However, following cessation of interferon treatment, approximately 50% of the responding patients relapsed, resulting in a “durable” response rate as assessed by normalization of serum ALT concentrations of approximately 20 to 25%. In addition, studies that have examined six months of type 1 interferon therapy using changes in HCV RNA values as a clinical endpoint have demonstrated that up to 35% of patients will have a loss of HCV RNA by the end of therapy (Tong et al., 1997, supra). However, as with the ALT endpoint, about 50% of the patients relapse six months following cessation of therapy resulting in a durable virologic response of only 12% (23). Studies that have examined 48 weeks of therapy have demonstrated that the sustained virological response is up to 25%.

Ribozymes in combination with IFN have the potential to improve the effectiveness of treatment of HCV or any of the other indications discussed above. Ribozymes targeting RNAs associated with diseases such as infectious diseases, autoimmune diseases, and cancer, can be used individually or in combination with other therapies such as IFN to achieve enhanced efficacy.

### EXAMPLES

The following are non-limiting examples showing the selection, isolation, synthesis and activity of enzymatic nucleic acids of the instant invention.

The following examples demonstrate the selection of ribozymes that cleave HCV RNA. The methods described herein represent a scheme by which ribozymes may be derived that cleave other RNA targets required for HCV replication.

### Example 1

Identification of Potential Ribozyme Cleavage Sites in HCV RNA

The sequence of HCV RNA was screened for accessible sites using a computer folding algorithm. Regions of the mRNA that did not form secondary folding structures and contained potential hammerhead and/or hairpin ribozyme cleavage sites were identified. The sequences of these cleavage sites are shown in Tables IV-VIII, and X.

### Example 2

Selection of Ribozyme Cleavage Sites in HCV RNA

To test whether the sites predicted by the computer-based RNA folding algorithm corresponded to accessible sites in HCV RNA, 20 hammerhead sites were selected for analysis. Ribozyme target sites were chosen by analyzing genomic sequences of HCV (Input Sequence=HPCJTA (Acc#D11168 & D01171)) and prioritizing the sites on the basis of folding. Hammerhead ribozymes were designed that could bind each target (see FIG. 1) and were individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the ribozyme sequences fold into the appropriate secondary structure. Those ribozymes with unfavorable intramolecular interactions between the binding arms and the catalytic core were eliminated from consideration. As noted below, varying binding arm lengths can be chosen to optimize activity. Generally, at least 5 bases on each arm are able to bind to, or otherwise interact with, the target RNA.

Selection of ribozyme candidates was initiated by scanning for all hammerhead cleavage sites in an HCV RNA sequence derived from a patient infected with HCV genotype 1b. The results of this sequence analysis are shown in Table III. As seen by Table III, 1300 hammerhead ribozyme sites were identified by this analysis. Next, in order to identify hammerhead ribozyme candidates that would cleave in the conserved regions of the HCV genome, a sequence alignment of approximately 50 HCV isolates from genotypes 1a, 1b, 2a, 2b, 2c, 3a, 3b, 4a, 5a, and 6 was completed. Within genotype sites were identified that are in areas having the greatest sequence identity between all isolates examined. This analysis reduced the hammerhead ribozyme candidates to about 23 (Table III).

Due to the high sequence variability of the HCV genome, selection of ribozymes for broad therapeutic applications should probably involve the conserved regions of the HCV genome. A list of the thirty-hammerhead ribozymes derived from the conserved regions (5′-Non-Coding Region (NCR), 5′-end of core protein coding region, and 3′-NCR) of the HCV genome is shown in Table IV. In general, ribozymes targeted to sites located in the 5′ terminal region of the HCV genome should block translation while ribozymes cleavage sites located in the 3′ terminal region of the genome should block RNA replication.

### Example 3

Chemical Synthesis and Purification of Ribozymes

Ribozymes of the hammerhead or hairpin motif were designed to anneal to various sites in the RNA message. The binding arms are complementary to the target site sequences described above. The ribozymes were chemically synthesized. The method of synthesis used followed the procedure for normal RNA synthesis as described in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were >98%.

Inactive hammerhead ribozymes were synthesized by substituting switching the order of G₅A₆ and substituting a U for A₁₄ (numbering from Hertel et al., 1992 Nucleic Acids Res., 20, 3252). Hairpin ribozymes were synthesized in two parts and annealed to reconstruct the active ribozyme (Chowrira and Burke, 1992 Nucleic Acids Res., 20, 2835-2840). Ribozymes were also synthesized from DNA templates using bacteriophage T7 RNA polymerase (Milligan and Uhlenbeck, 1989, Methods Enzymol. 180, 51). Ribozymes were modified to enhance stability by modification with nuclease resistant groups, for example, 2′-amino, 2′-C-allyl, 2′-flouro, 2′-O-methyl, 2′-H (for a review, see Usman and Cedergren, 1992 TIBS 17, 34). Ribozymes were purified by gel electrophoresis using general methods or were purified by high pressure liquid chromatography (HPLC; see Wincott et al., supra; the totality of which is hereby incorporated herein by reference) and were resuspended in water. The sequences of the chemically synthesized ribozymes used in this study are shown below in Tables IV-X.

### Example 4

Ribozyme Cleavage of HCV RNA Target in vitro

Ribozymes targeted to the HCV are designed and synthesized as described above.

These ribozymes can be tested for cleavage activity in vitro, for example, using the following procedure. The target sequences and the nucleotide location within the HCV are given in Table IV.

Cleavage Reactions: Full-length or partially full-length, internally-labeled target RNA for ribozyme cleavage assay is prepared by in vitro transcription in the presence of [-³²p] CTP, passed over a G 50Sephadex® column by spin chromatography and used as substrate RNA without further purification. Alternately, substrates are 5′³²p-end labeled using T4 polynucleotide kinase enzyme. Assays are performed by pre-warming a 2×concentration of purified ribozyme in ribozyme cleavage buffer (50 mM Tris-HCl, pH 7.5 at 37° C., 10 mM MgCl₂) and the cleavage reaction was initiated by adding the 2×ribozyme mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM ribozyme, i.e., ribozyme excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imager® quantitation of bands representing the intact substrate and the cleavage products.

### Example 5

Ability of HCV Ribozymes to Cleave HCV RNA in Patient Serum

Ribozymes targeting sites in HCV RNA were synthesized using modifications that confer nuclease resistance (Beigelman, 1995, J. Biol. Chem. 270, 25702). It has been well documented that serum from chronic hepatitis C patients contains on average 3×10⁶ copies/ml of HCV RNA. To further select ribozyme product candidates, the 30 HCV specific ribozymes are characterized for HCV RNA cleavage activity utilizing HCV RNA isolated from the serum of genotype 1b HCV patients. The best candidates from the HCV genotype 1b screen will be screened against isolates from the wide range of HCV genotypes including 1a, 1b, 2a, 2b, 2c, 3a, 3b, 4a, 5a, and 6. Therefore, it is possible to select ribozyme candidates for further development based on their ability to broadly cleave HCV RNA from a diverse range of HCV genotypes and quasi-species.

### Example 6

Ribozyme Cleavage of Conserved HCV RNA Target Sites in vitro

There are three regions of the genome that are highly conserved, both within a genotype and across different genotypes. These conserved sequences occur in the 5′ and 3′ non-coding regions (NCRs) as well as the 5′-end of the Core Protein coding region. These regions are thought to be important for HCV RNA replication and translation. Thus, therapeutic agents that target these conserved HCV genomic regions may have a significant impact over a wide range of HCV genotypes. The presence of quasi-species, and the potential for infection with more than one genotype makes this a critical feature of an effective therapy. Moreover, it is unlikely that drug resistance will occur, since mutations that have been suggested to lead to drug resistance typically do not occur within these highly conserved regions. In order to target multiple genotypes and decrease the chance of developing drug resistance, Applicant has designed ribozymes that cleave in regions of identity within the conserved regions discussed above.

Sequence alignments were performed for the 5′ NCR, the 5′ end of the Core Protein coding region, and the 3′ NCR. For the 5′ NCR, 34 different isolates representing genotypes 1a, 1b, 2a, 2b, 2c, 3a, 3b, 4a, 4f, and 5a were aligned. The alignments included the sequences from nucleotide position 1 to nucleotide position 350 (18 nucleotides downstream of the initiator ATG codon), using the reported sequence “HPCK1S1” as the reference for numbering. For the Core Protein coding region, 44 different isolates representing genotypes 1a, 1b, 2a, 2b, 2c, 3a, 3b, 4a, 4c, 4f, 5a, and 6a were aligned. These alignments included 600 nucleotides, beginning 8 nucleotides upstream of the initiator ATG codon. As the reference for numbering, the reported sequence “HPCCOPR” was used, with the “C” eight nucleotides upstream of the initiator codon ATG designated as “1”. For the 3′ NCR region, 20 different isolates representing genotypes 1b, 2a, 2b, 3a, and 3b were aligned. These alignments included sequences in the 3′ terminal 235 nucleotides of the genome, with the reported sequence “D85516” used as the reference for numbering, and the 235^{th} nucleotide from the 3′ end designated as “1”.

During analysis of the alignments of each region, each sequence was compared to the respective reference sequence (identified above), and regions of identity across all isolates were determined. All potential ribozyme sites were identified in the reference sequence. The highest priority for choosing ribozyme sites was that the site should have 100% identity across all isolates aligned, at every position in both the cleavage site and binding arms. Ribozyme sites that met these criteria were chosen. In addition, two specific allowances were made as follows. 1) If a potential ribozyme site had 100% sequence identity at all except one or two nucleotide positions, then the actual nucleotide at that position was examined in the isolate(s) that differed. If that nucleotide was such that a ribozyme designed to allow “G:U wobble” base-paring could function on all the isolates, then that site was chosen. 2) If a potential ribozyme site had 100% sequence identity at all except one or two nucleotide positions, then the genotype of the isolate which contained the differing nucleotide(s) was examined. If the genotype of the isolate that differed was of extremely rare prevalence, then that site was also chosen.

Ribozyme sites identified and referred to below use the following nomenclature: “region of the genome in which the site exists” followed by “nucleotide position 5′ to the cleavage site” (according to the reference sequence and numbering described above). For example, a ribozyme cleavage site at nucleotide position 67 in the 5′ NCR is designated “5-67”, and a ribozyme cleavage site at position 48 in the core coding region is designated “c48”.

A number of these ribozymes were screened in an in vitro HCV cleavage assay to select appropriate ribozyme candidates for cell culture studies. The ribozymes selected for screening targeted the 5′ UTR region that is necessary for HCV translation. These sites are all conserved among the 8 major HCV genotypes and 18 subtypes, and have a high degree of homology in every HCV isolate that was used in the analysis described above. HCV RNA of four different genotypes (1b, 2a, 4, and 5) were isolated from human patients and the 5′ HCV UTR and 5′ core region were amplified using RT-PCR. Run-off transcripts of the 5′ HCV UTR region (˜750 nt transcripts) were prepared from the RT-PCR products, which contained a T7 promoter, using the T7 Megascript® transcription kit and the manufacturers protocol (Ambion, Inc.). Unincorporated nucleotides are removed by spin column filtration on Bio-Gel P-60 resin (Bio-Rad). The filtered transcript was 5′ end labeled with ³²P using Polynucleotide Kinase (Boehringer/Mannheim) and 150μCi/μl Gamma-32P-ATP (NEN) using the enzyme manufacturer's protocol. The kinased transcript is spin purified again to remove unincorporated Gamma-32P-ATP and gel purified on 5% polyacrylamide gel.

Ribozymes targeting various sites from Table IV were selected and tested on the 5′ HCV UTR transcript sequence to test the efficiency of RNA cleavage. 15 ribozymes were synthesized as previously described (Wincott et al., supra).

Assays were performed by pre-warming a 2×(2 μM) concentration of purified ribozyme in ribozyme cleavage buffer (50 mM TRIS pH 7.5, 10 mM MgCl₂, 10 units RNase Inhibitor (Boehringer/Mannheim), 10 mM DTT, 0.5μg tRNA) and the cleavage reaction was initiated by adding the 2×ribozyme mix to an equal volume of substrate RNA (17.46 pmole final concentration) that was also pre-warmed in cleavage buffer. The assay was carried out for 24 hours at 37° C. using a final concentration of 1 μM ribozyme, i.e., ribozyme excess. The reaction was quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imager® quantitation of bands representing the intact substrate and the cleavage products.

Observed cleavage fragment sizes from the gels are correlated to predicted fragment sizes by comparison to the RNA marker. The optical density of expected cleavage fragments are determined from the phosphorimage plates and ranked from highest density, indicating the most cleavage product, to lowest of each genotype of HCV transcript tested. The top 3 cleaving ribozymes (out of 15 ribozymes tested) are given ranking values of 5, the next 3 highest densities are given ranking values of 4, etc. for every genotype tested. The ranking values for each ribozyme are averaged between the genotypes tested. Individual and average ribozyme ranking values are graphed and compared. The results (FIG. 2) demonstrate that many of these tested ribozymes are able to give high levels of cleavage regardless of genotype. In particular, ribozymes targeting site HCV0.5-258, HCV0.5-294, HCV0.5-313 (Sakamoto et al., J. Clinical Investigation 1996 98(12):2720-2728), and HCV0.5-318 (Table IV) appear to demonstrate a consistent pattern of RNA cleavage

### Example 7

Inhibition of Luciferase Activity Using HCV Targeting Ribozymes in OST7 Cells

The capability of ribozymes to inhibit HCV RNA intracellularly was tested using a dual reporter system that utilizes both firefly and Renilla luciferase (FIG. 3).

The ribozymes targeted to the 5′ HCV UTR region, which when cleaved, would prevent the translation of the transcript into luciferase. OST-7 cells were plated at 12,500 cells per well in black walled 96 well plates (Packard) in medium DMEM containing 10% fetal bovine serum, 1% pen/strep, and 1% L-glutamine and incubated at 37° C. overnight.

A plasmid containing T7 promoter expressing 5′ HCV UTR and firefly luciferase (T7C1-341 (Wang et al., 1993, J. of Virol. 67, 3338-3344)) was mixed with a pRLSV40 Renilla control plasmid (Promega Corporation) followed by ribozyme, and cationic lipid to make a 5×concentration of the reagents (T7C1-341 (4 μg/ml), pRLSV40 renilla luciferase control (6 μg/ml), ribozyme (250 nM), transfection reagent (28.5 μg/ml).

The complex mixture was incubated at 37° C. for 20 minutes. The media was removed from the cells and 120 μl of Opti-mem media was added to the well followed by 30 μl of the 5×complex mixture. 150 μl of Opti-mem was added to the wells holding the untreated cells. The complex mixture was incubated on OST-7 cells for 4 hours, lysed with passive lysis buffer (Promega Corporation) and luminescent signals were quantified using the Dual Luciferase Assay Kit using the manufacturer's protocol (Promega Corporation). The ribozyme sequences used are given in Table IV. The ribozymes used were of the hammerhead motif. The hammerhead ribozymes were chemically modified such that the ribozyme consists of ribose residues at five positions (see for example FIG. 7); position 4 has either 2′-C-allyl or 2′-amino modification; position 7 has either 2′-amino modification or 2-O-methyl modification; the remaining nucleotide positions contain 2′-O-methyl substitutions; four nucleotides at the 5′ terminus contains phosphorothioate substitutions. Additionally, the 3′ end of the ribozyme includes a 3′-3′ linked inverted abasic moiety (abasic deoxyribose; iH). The data (FIG. 4) is given as a ratio between the firefly and Renilla luciferase fluorescence. All of the ribozymes targeting 5′ HCV UTR were able to reduce firefly luciferase signal relative to renilla luciferase.

### Example 9

Ribozyme Mediated Inhibition of Luciferase Activity Compared to Its Inactive Control in OST-7 Cells

The dual reporter system described above was utilized to determine the level of reduction of luciferase activity mediated by a ribozyme compared to its inactive control. Ribozymes, having the chemical composition described in the previous example, to sites HCV 313 and 318 (Table IV) and their inactive controls were synthesized as above. The inactive control has the same nucleotide base composition as the active ribozyme but the nucleotide sequence has been scrambled. The protocols utilized for tissue culture and the luciferase assay was exactly as given in Example 8 except the ribozyme concentration in the 5×complex mixture was 1 mM (final concentration on the cells was 200 nM).

The results are given in FIG. 5. The ribozyme targeting HCV.5-318 was able to greatly reduce firefly luciferase activity compared to the untreated and inactive controls. The ribozyme targeting HCV.5-313 was able to slightly reduce firefly luciferase activity compared to the inactive control.

### Example 10

Ribozyme Inhibition of Viral Replication

During HCV infection, viral RNA is present as a potential target for ribozyme cleavage at several processes: uncoating, translation, RNA replication and packaging. Target RNA may be more or less accessible to ribozyme cleavage at any one of these steps. Although the association between the HCV initial ribosome entry site (IRES) and the translation apparatus is mimicked in the HCV 5′UTR/luciferase reporter system (Example 9), these other viral processes are not represented in the OST7 system. The resulting RNA/protein complexes associated with the target viral RNA are also absent. Moreover, these processes may be coupled in an HCV-infected cell which could further impact target RNA accessibility. Therefore, we tested whether ribozymes designed to cleave the HCV 5′UTR could effect a replicating viral system.

Recently, Lu and Wimmer characterized an HCV-poliovirus chimera in which the poliovirus IRES was replaced by the IRES from HCV (Lu & Wimmer, 1996, Proc. Nat. Acad. Sci. USA. 93, 1412-1417). Poliovirus (PV) is a positive strand RNA virus like HCV, but unlike HCV is non-enveloped and replicates efficiently in cell culture. The HCV-PV chimera expresses a stable, small plaque phenotype relative to wild type PV.

The following ribozymes were synthesized for the experiment (Table VIII): ribozyme targeting site 183 (3 5′-end phosphorothioate linkages), scrambled control to site 183, ribozyme to site 318 (3 5′-end phosphorothioate linkages), ribozyme targeting site 183 (4 5′-end phosphorothioate linkages), inactive ribozyme targeting site 183 (4 5′-end phosphorothioate linkages). HeLa cells were infected with the HCV-PV chimera for 30 minutes and immediately treated with ribozyme. HeLa cells were seeded in U-bottom 96-well plates at a density of 9000-10,000 cells/well and incubated at 37° C. under 5% CO₂ for 24 h. Transfection of ribozyme (200 nM) was achieved by mixing of 10×ribozyme (2000 nM) and 10×of a cationic lipid (80 μg/ml) in DMEM (Gibco BRL) with 5% fetal bovine serum (FBS). Ribozyme/lipid complexes were allowed to incubate for 15 minutes at 37° C. under 5% CO₂. Medium was aspirated from cells and replaced with 80 μls of DMEM (Gibco BRL) with 5% FBS serum, followed by the addition of 20 μls of 10×complexes. Cells were incubated with complexes for 24 hours at 37° C. under 5% CO₂.

The yield of HCV-PV from treated cells (FIG. 6A) was quantified by plaque assay. The plaque assays were performed by diluting virus samples in serum-free DMEM (Gibco BRL) and applying 100 μl to HeLa cell monolayers (˜80% confluent) in 6-well plates for 30 minutes. Infected monolayers were overlayed with 3 ml 1.2% agar (Sigma) and incubated at 37° C. under 5% CO₂. Two-three days later the overlay was removed, monolayers were stained with 1.2% crystal violet, and plaque forming units were counted. The data is shown in FIG. 6A. Ribozymes to site 183 inhibited HCV-PV replication by >80% (P<0.05) compared to the scrambled control (FIG. 6A, first two bars). In addition, 3 or 4 phosphorothioate stabilization was equally effective (P<0.05 vs. control for each) in inhibiting viral replication (compare 1^{st} and 4^{th} bar in FIG. 6A). Ribozymes to the 318 site also had a statistically significant (P<0.05), effect on viral replication (compare 2^{nd} and ₃^{rd} bar in FIG. 6A).

To confirm that a ribozyme cleavage mechanism was responsible for the inhibition of HCV-PV replication observed, HCV-PV infected cells were treated with ribozymes to site 183 that maintained binding arm sequences but contained a mutation in the catalytic core to attenuate cleavage activity (Table I). Viral replication in these cells was not inhibited compared to cells treated with the scrambled control ribozyme (FIG. 6A, 4^{th} and 5^{th} bar), indicating that ribozyme cleavage activity was required for the inhibition of HCV-PV replication observed. In addition, ribozymes targeting site 183 of the HCV 5′UTR had no effect on wild type PV replication (FIG. 6B). These data provide evidence that the ribozyme-mediated inhibition of HCV-PV replication was dependent upon the HCV 5′UTR and not a general inhibition of PV replication.

Ribozymes to site 183 were also tested for the ability to inhibit HCV-PV replication during a single infectious cycle in HeLa cells (FIG. 8). Cells treated with ribozyme to site 183 (7/4 format) produced significantly less virus than cells treated with the scrambled control (>80% inhibition at 8h post infection, P<0.001).

### Example 11

Shortening of Ribozyme Lengths

All the ribozymes described in example 10 above contained 7 nucleotides on each binding arms and contained a 4 base-paired stem II element (7/4 format). For pharmaceutical manufacture of a therapeutic ribozyme it is advantageous to minimize sequence length if possible. Thus ribozymes to site 183 were shortened by removing the outer most nucleotide from each binding arm such that the ribozyme has six nucleotides in each binding arm and the stem II region is four base-paired long (6/4 format); removing one base-pair (2 nucleotides) in stem II resulting in a 3 base-paired stem II (7/3 format); or removing one nucleotide from each binding arm and shortening the stem II by one base-pair (6/3 format). (See FIG. 7 for a schematic representation of each of these ribozymes). Ribozymes in all tested formats gave significant inhibition of viral replication (FIG. 8) with the 7/4, 7/3 and 6/3 formats being almost identical at the 8 h timepoint (P<0.001 across time course for all formats). The shortest ribozyme tested (6/3 format) was slightly more efficacious (>90% inhibition, P<0.001) than the 7/4 ribozyme (˜80% inhibition, P<0.001). The 6/3 ribozyme may have a greater ability to access site 183 in the HCV-PV chimera.

### Example 12

Combination Therapy of HCV Ribozymes and Interferon

HeLa cells (10,000 cells per well) were pre-treated with 12.5 Units/ml of Interferon alpha in complete media (DMEM+5% FBS) or pre-treated with complete media alone for 4 hours and then infected with HCV-PV at an MOI=0.1 for 30 minutes. The viral inoculum was then removed and 200 nM ribozyme targeted to HCV site 183 (Rz) or binding attenuated control, which has mutations in the catalytic core of the ribozyme that severely attenuates the activity of the ribozyme, (BAC) was delivered using cationic lipid in complete media for 24 hours. After 24 hours, the cells were lysed three times by freeze/thaw to release virus and virus was quantified by plaque assay. Viral yield is shown as mean plaque forming units per ml (pfu/ml)+SEM. The data is shown in FIG. 10.

Pre-treatment with interferon (IFN) reduces the viral yield by ˜10⁻¹ in control treated cells (BAC+IFN versus BAC). Ribozyme treated cells produce 2×10⁻¹ less virus than control-treated cells (Rz versus BAC). The combination of Rz and IFN treatment results in a synergistic 4×10⁻² reduction in viral yield (Rz+IFN versus BAC). An additive effect would result in only a 3×10⁻¹ reduction (1×10⁻¹+2×10⁻¹).

### Example 13

Inhibition of Hepatitis C Virus Using Various Ribozyme Motifs

A number of varying ribozyme motifs (RPI motifs I-III; FIG. 9), were tested for their ability to inhibit HCV propagation in tissue culture. An example of RPI motif I (G-cleaver) is described in Kore et al., 1998, Nucleic Acids Research 26, 4116-4120, while an example of RPI motif II (Inozyme) is described in Ludwig & Sproat, International PCT Publication No. WO 98/58058). RPI motif III is a new ribozyme motif which applicant has recently developed and an example of this motif was tested herein.

OST7 cells were maintained in Dulbecco's modified Eagle's medium (GIBCO BRL) supplemented with 10% fetal calf serum, L-glutamine (2 mM) and penicillin/streptomycin. For transfections, OST7 cells were seeded in black-walled 96-well plates (Packard Instruments) at a density of 12,500 cells/well and incubated at 37° C. under 5% CO₂ for 24 hours. Co-transfection of target reporter HCVT7 C (0.8 g/ml), control reporter pRLSV40, (1.2 μg/ml) and ribozyme, 50-200 nM was achieved by the following method: a 5×mixture of HCVT7 C (4 μg/ml), pRLSV40 (6 μg/ml), ribozyme (250-1000 nM) and cationic lipid (28.5 μg/ml) was made in 150 μls of OPTI-MEM (GIBCO BRL) minus serum. Reporter/ribozyme/lipid complexes were allowed to form for 20 minutes at 37° C. under 5% CO₂. Medium was aspirated from OST7 cells and replaced with 120 μls of OPTI-MEM (GIBCO BRL) minus serum, immediately followed by the addition of 30 μls of 5×reporter/ribozyme/lipid complexes. Cells were incubated with complexes for 4 hours at 37° C. under 5% CO₂ . Luciferase assay was performed as described in example 7. The data is summarized in Table IX, with each motif's results listed along with its control. All of the ribozyme motifs were able to reduce the amount of HCV produced by the cells compared to the ribozymes not targeted to any HCV (irrelevant controls).

### Example 14

General Protocol for Virus Infection and Ribozyme Delivery

HeLa cells were seeded in 96-well plates at a density of 9000-10,000 cells/well and incubated at 37° C. under 5% CO₂ for 24 h. Cells were infected with HCV-PV at an MOI−0.1 for 30 min. Transfection of ribozyme or control oligonucleotides (200 nM final) was achieved by mixing of 5×ribozyme or control oligonucleotides (1000 nM) and 5×cationic lipid (40 μg/ml at 5×, 800 ng/well final) in DMEM with 5% fetal bovine serum (FBS) in U-bottom 96-well plates. Ribozyme/lipid complexes were allowed to incubate for 15 min at 37° C. under 5% CO₂. Medium was aspirated from cells and replaced with 80 μl of DMEM with 5% FBS serum, followed by the addition of 20 μl of 5×complexes. Cells were incubated with complexes for 24 h at 37° C. under 5% CO₂. After 24 h cells were lysed by three freeze/thaw cycles to release virus and virus was quantified by plaque assay.

### Example 15

General Protocol for HCV Plaque Assay

Virus samples were diluted in serum-free DMEM and 100 μl applied to HeLa cell monolayers (˜80% confluent) in 6-well plates for 30 min. Infected monolayers were overlayed with 3 ml 1.2% agar (Sigma, St. Louis, Mo.) and incubated at 37° C. under 5% CO₂. When plaques were visible (after two to three days) the overlay was removed, monolayers were stained with 1.2% crystal violet, and plaque forming units were counted.

### Example 16

Inhibition of Hepatitis C Virus Using Other Ribozyme Directed Against the HCV Minus Strand

HeLa cells in 96-well plates were infected with a chimeric Hepatitis C-Poliovirus (HCV-PV) at a multiplicity of infection (MOI) of 0.1. Virus inoculum was then replaced with media containing 5% serum and 200 nM ribozyme (Table X) or scrambled attenuated control (SAC), as indicated, complexed to cationic lipid. After 24 h cells were lysed 3 times by freeze/thaw and virus was quantified by plaque assay. Results are summarized in FIGS. 12 and 13. Plaque forming units (pfu)/ml are shown as the mean of triplicate samples+S.D.

### Example 17

Dose Response of Ribozyme Directed Against the HCV Minus Strand

Cells were infected and treated with ribozyme as described in Example 16 except that various amounts (as indicated) of anti-HCV ribozyme RPI.15006 was mixed with a control oligonucleotide (SAC) to maintain a constant 200 nM total dose of nucleic acid for delivery. FIG. 14 shows the results of this study that demonstrates an effective dose response in cells to treatment with a ribozyme directed against the HCV minus strand.

### Example 18

Dose Response of Ribozyme Directed Against the HCV Plus Strand Combined with Ribozymes Targeting the HCV Minus Strand

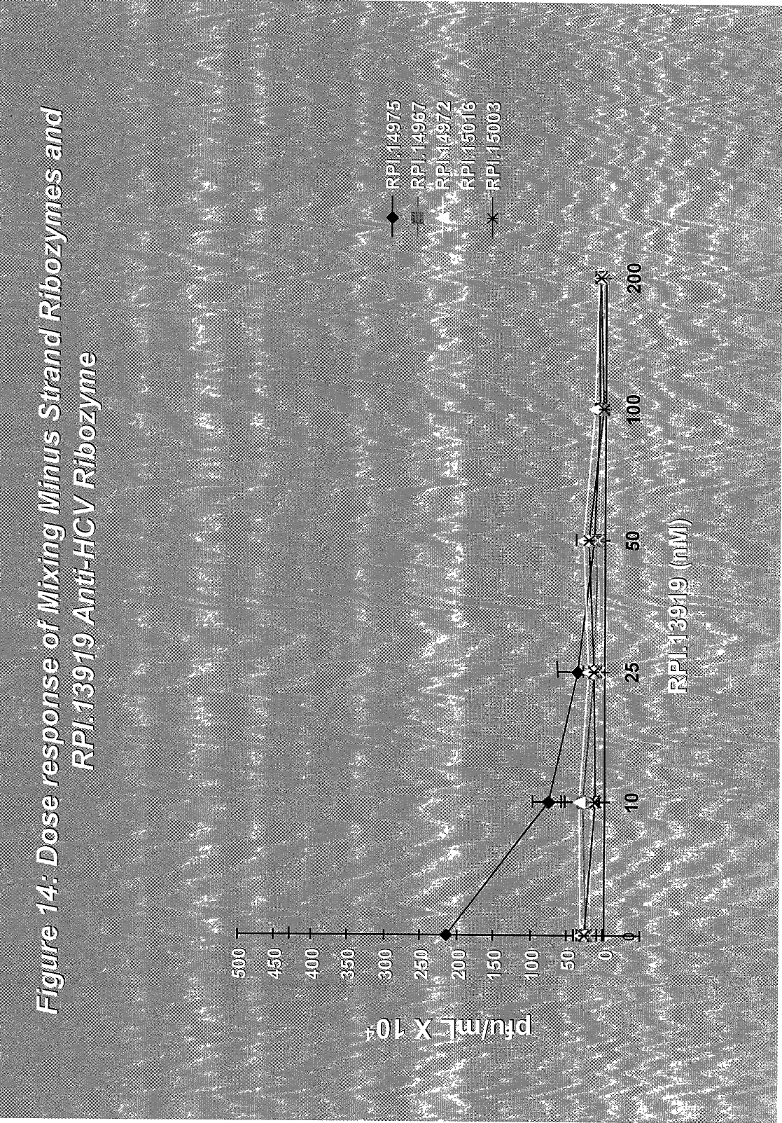

Cells were infected and treated with ribozyme as described in Example 16 except that various amounts (as indicated) of anti-HCV ribozyme RPI.13919, targeting the plus strand, was mixed with ribozymes targeting the minus strand, as noted, to maintain a constant 200 nM total dose of nucleic acid for delivery. FIG. 15 shows the results of this study that demonstrates an effective dose response in cells to treatment with a ribozyme (RPI 13919) directed against the HCV plus strand combined with a ribozyme targeting the HCV minus strand (RPI 14975).

### Example 19

Inhibition of HCV in vivo

Ribozyme directed reduction of HCV in vivo was examined in a mouse model, generally described in Vierling, International PCT Publication No. WO 99/16307, using HCV RNA as an endpoint. The study compared mice treated with ribozymes compared to scrambled-attenuated-core ribozymes (SAC) and saline controls. Active ribozyme and SAC were dosed from day 5 through 20 post-transplant. Various modes of analysis were used including ANOVA of raw quantitative HCV RNA, Dunnett's of raw quantitative HCV RNA, ANOVA of log10 quantitative HCV RNA, Dunnett's of log₁₀ quantitative HCV RNA, and Chi Square of qualitative results (HCV RNA +/−). Treatment with active ribozyme (RPI 13918), resulted in significant reduction of HCV RNA at 12 and 16 days using quantitative analysis (p<0.05 by Dunnett's using the log₁₀ transformed HCV RNA results for all observations). The use of qualitative assessment, by converting the quantitative data into positive or negative results, confirmed with same trend. This study suggests that treatment with active anti-HCV ribozymes results in a significant reduction in HCV RNA in a trimeric mouse model.

Cell Culture Assays

Although there have been reports of replication of HCV in cell culture (see below), these systems are difficult to replicate and have proven unreliable. Therefore, as was the case for development of other anti-HCV therapeutics such as interferon and ribavirin, after demonstration of safety in animal studies applicant can proceed directly into a clinical feasibility study.

Several recent reports have documented in vitro growth of HCV in human cell lines (Mizutani et al., Biochem Biophys Res Commun 1996 227(3):822-826; Tagawa et al., Journal of Gasteroenterology and Hepatology 1995 10(5):523-527; Cribier et al., Journal of General Virology 76(10):2485-2491; Seipp et al., Journal of General Virology 1997 78(10)2467-2478; lacovacci et al., Research Virology 1997 148(2):147-151; Iocavacci et al., Hepatology 1997 26(5) 1328-1337; Ito et al., Journal of General Virology 1996 77(5):1043-1054; Nakajima et al, Journal of Virology 1996 70(5):3325-3329; Mizutani et al., Journal of Virology 1996 70(10):7219-7223; Valli et al., Res Virol 1995 146(4): 285-288; Kato et al., Biochem Biophys Res Comm 1995 206(3):863-869). Replication of HCV has been demonstrated in both T and B cell lines as well as cell lines derived from human hepatocytes. Demonstration of replication was documented using either RT-PCR based assays or the b-DNA assay. It is important to note that the most recent publications regarding HCV cell cultures document replication for up to 6-months.

In addition to cell lines that can be infected with HCV, several groups have reported the successful transformation of cell lines with cDNA clones of full-length or partial HCV genomes (Harada et al., Journal of General Virology 1995 76(5)1215-1221; Haramatsu et al., Journal of Viral Hepatitis 1997 4S(1):61-67; Dash et al., American Journal of Pathology 1997 151(2):363-373; Mizuno et al., Gasteroenterology 1995 109(6):1933-40; Yoo et al., Journal Of Virology 1995 69(1):32-38).

Animal Models

The best characterized animal system for HCV infection is the chimpanzee.

Moreover, the chronic hepatitis that results from HCV infection in chimpanzees and humans is very similar. Although clinically relevant, the chimpanzee model suffers from several practical impediments that make use of this model difficult. These include; high cost, long incubation requirements and lack of sufficient quantities of animals. Due to these factors, a number of groups have attempted to develop rodent models of chronic hepatitis C infection. While direct infection has not been possible several groups have reported on the stable transfection of either portions or entire HCV genomes into rodents (Yamamoto et al., Hepatology 1995 22(3): 847-855; Galun et al., Journal of Infectious Disease 1995 172(1):25-30; Koike et al., Journal of General Virology 1995 76(12)3031-3038; Pasquinelli et al., Hepatology 1997 25(3): 719-727; Hayashi et al., Princess Takamatsu Symp 1995 25:1430149; Mariya K, Yotsuyanagi H, Shintani Y, Fujie H, Ishibashi K, Matsuura Y, Miyamura T, Koike K. Hepatitis C virus core protein induces hepatic steatosis in transgenic mice. Journal of General Virology 1997 78(7) 1527-1531; Takehara et al., Hepatology 1995 21(3):746-751; Kawamura et al., Hepatology 1997 25(4): 1014-1021). In addition, transplantation of HCV infected human liver into immunocompromised mice results in prolonged detection of HCV RNA in the animal's blood.

Vierling, International PCT Publication No. WO 99/16307, describes a method for expressing hepatitis C virus in an in vivo animal model. Viable, HCV infected human hepatocytes are transplanted into a liver parenchyma of a scid/scid mouse host.

The scid/scid mouse host is then maintained in a viable state, whereby viable, morphologically intact human hepatocytes persist in the donor tissue and hepatitis C virus is replicated in the persisting human hepatocytes. This model provides an effective means for the study of HCV inhibition by ribozymes in vivo.

Diagnostic Uses

Ribozymes of this invention may be used as diagnostic tools to examine genetic drift and mutations within diseased cells or to detect the presence of HCV RNA in a cell. The close relationship between ribozyme activity and the structure of the target RNA allows the detection of mutations in any region of the molecule, which alters the base-pairing and three-dimensional structure of the target RNA. By using multiple ribozymes described in this invention, one may map nucleotide changes, which are important to RNA structure and function in vitro, as well as in cells and tissues. Cleavage of target RNAs with ribozymes may be used to inhibit gene expression and define the role (essentially) of specified gene products in the progression of disease. In this manner, other genetic targets may be defined as important mediators of the disease. These experiments will lead to better treatment of the disease progression by affording the possibility of combination therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention are well known in the art, and include detection of the presence of mRNAs associated with HCV related condition. Such RNA is detected by determining the presence of a cleavage product after treatment with a ribozyme using standard methodology.

In a specific example, ribozymes which can cleave only wild-type or mutant forms of the target RNA are used for the assay. The first ribozyme is used to identify wild-type RNA present in the sample and the second ribozyme can be used to identify mutant RNA in the sample. As reaction controls, synthetic substrates of both wild-type and mutant RNA can be cleaved by both ribozymes to demonstrate the relative ribozyme efficiencies in the reactions and the absence of cleavage of the “non-targeted” RNA species. The cleavage products from the synthetic substrates can also serve to generate size markers for the analysis of wild-type and mutant RNAs in the sample population. Thus each analysis can involve two ribozymes, two substrates and one unknown sample which will be combined into six reactions. The presence of cleavage products can be determined using an RNase protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., HCV) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

Additional Uses

Potential usefulness of sequence-specific enzymatic nucleic acid molecules of the instant invention might have many of the same applications for the study of RNA that DNA restriction endonucleases have for the study of DNA (Nathans et al., 1975 Ann. Rev. Biochem. 44:273). For example, the pattern of restriction fragments could be used to establish sequence relationships between two related RNAs, and large RNAs could be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

### TABLE I

Characteristics of Naturally Occurring Ribozymes

Group I Introns

Size: ˜150 to >1000 nucleotides.

Requires a U in the target sequence immediately 5′ of the cleavage site.

Binds 4-6 nucleotides at the 5′-side of the cleavage site.

Reaction mechanism: attack by the 3′-OH of guanosine to generate cleavage products with 3′-OH and 5′-guanosine.

Additional protein cofactors required in some cases to help folding and maintainance of the active structure.

Over 300 known members of this class. Found as an intervening sequence in Tetrahymena thermophila rRNA, fungal mitochondria, chloroplasts, phage T4, blue-green algae, and others.

Major structural features largely established through phylogenetic comparisons, mutagenesis, and biochemical studies [ⁱ,ⁱ].

Complete kinetic framework established for one ribozyme [ⁱⁱⁱ,^{iv},^{v},^{vi}].

Studies of ribozyme folding and substrate docking underway [^{vii},^{viii},^{ix}].

Chemical modification investigation of important residues well established [^{x},^{xi}].

The small (4-6 nt) binding site may make this ribozyme too non-specific for targeted RNA cleavage, however, the Tetrahymena group I intron has been used to repair a “defective” -galactosidase message by the ligation of new -galactosidase sequences onto the defective message [^{xii}].

RNAse P RNA (M1 RNA)

Size: ˜290 to 400 nucleotides.

RNA portion of a ubiquitous ribonucleoprotein enzyme.

Cleaves tRNA precursors to form mature tRNA [^{xiii}].

Reaction mechanism: possible attack by M²⁺-OH to generate cleavage products with 3′-OH and 5′-phosphate.

RNAse P is found throughout the prokaryotes and eukaryotes. The RNA subunit has been sequenced from bacteria, yeast, rodents, and primates.

Recruitment of endogenous RNAse P for therapeutic applications is possible through hybridization of an External Guide Sequence (EGS) to the target RNA [^{xiv},^{xv}]

Important phosphate and 2′ OH contacts recently identified [^{xvi},^{xvii}]

Group II Introns

Size: >1000 nucleotides.

Trans cleavage of target RNAs recently demonstrated [^{xviii},^{xix}]

Sequence requirements not fully determined.

Reaction mechanism: 2′-OH of an internal adenosine generates cleavage products with 3′-OH and a “lariat” RNA containing a 3′-5′ and a 2′-5′ branch point.

Only natural ribozyme with demonstrated participation in DNA cleavage [^{xx},^{xxi}] in addition to RNA cleavage and ligation.

Major structural features largely established through phylogenetic comparisons [^{xxii}].

Important 2′ OH contacts beginning to be identified [^{xxiii}]

Kinetic framework under development [^{xxiv}]

Neurospora VS RNA

Size: ˜144 nucleotides.

Trans cleavage of hairpin target RNAs recently demonstrated [^{xxv}].

Sequence requirements not fully determined.

Reaction mechanism: attack by 2′-OH 5′ to the scissile bond to generate cleavage products with 2′,3′-cyclic phosphate and 5′-OH ends.

Binding sites and structural requirements not fully determined.

Only 1 known member of this class. Found in Neurospora VS RNA.

Hammerhead Ribozyme

(see text for references)

Size: ˜13 to 40 nucleotides.

Requires the target sequence UH immediately 5′ of the cleavage site.

Binds a variable number nucleotides on both sides of the cleavage site.

Reaction mechanism: attack by 2′-OH 5′ to the scissile bond to generate cleavage products with 2′,3′-cyclic phosphate and 5′-OH ends.

14 known members of this class. Found in a number of plant pathogens (virusoids) that use RNA as the infectious agent.

Essential structural features largely defined, including 2 crystal structures [^{xxvi},^{xxvii}]

Minimal ligation activity demonstrated (for engineering through in vitro selection) [^{xxviii}]

Complete kinetic framework established for two or more ribozymes [^{xxix}]

Chemical modification investigation of important residues well established [^{xxx}].

Hairpin Ribozyme

Size: ˜50 nucleotides.

Requires the target sequence GUC immediately 3′ of the cleavage site.

Binds 4-6 nucleotides at the 5′-side of the cleavage site and a variable number to the 3′-side of the cleavage site.

Reaction mechanism: attack by 2′-OH 5′ to the scissile bond to generate cleavage products with 2 ′,3′-cyclic phosphate and 5′-OH ends.

3 known members of this class. Found in three plant pathogen (satellite RNAs of the tobacco ringspot virus, arabis mosaic virus and chicory yellow mottle virus) which uses RNA as the infectious agent.

Essential structural features largely defined [^{xxxi},^{xxxii},^{xxxiii},^{xxxiv}]

Ligation activity (in addition to cleavage activity) makes ribozyme amenable to engineering through in vitro selection [^{xxxv}]

Complete kinetic framework established for one ribozyme [^{xxxvi}].

Chemical modification investigation of important residues begun [^{xxxvii},^{xxxviii}]

Hepatitis Delta Virus (HDV) Ribozym

Size: ˜60 nucleotides.

Trans cleavage of target RNAs demonstrated [^{xxxix}].

Binding sites and structural requirements not fully determined, although no sequences 5′ of cleavage site are required. Folded ribozyme contains a pseudoknot structure [^{xl}].

Reaction mechanism: attack by 2′-OH 5′ to the scissile bond to generate cleavage products with 2 ′,3′-cyclic phosphate and 5′-OH ends.

Only 2 known members of this class. Found in human HDV.

Circular form of HDV is active and shows increased nuclease stability [^{xh}]

TABLE II
2.5 μmol RNA Synthesis Cycle

| | | | | Wait |
|---|---|---|---|---|
| Reagent | Equivalents | Amount | | Time* |
| Phosphoramidites | 6.5 | 163 | μL | 2.5 |
| S-Ethyl Tetrazole | 23.8 | 238 | μL | 2.5 |
| Acetic Anhydride | 100 | 233 | μL | 5 sec |
| N-Methyl Imidazole | 186 | 233 | μL | 5 sec |
| TCA | 83.2 | 1.73 | mL | 21 sec |
| Iodine | 8.0 | 1.18 | mL | 45 sec |
| Acetonitrile | NA | 6.67 | mL | NA |

*Wait time does not include contact time during delivery.

TABLE III
Ribozyme Selection Characteristics

| Characteristic | Number |
|---|---|
| HCV Genome Length | 9436 kb |
| All Hammerhead Cleavage Sites* | 1300 |
| Conserved Region Hammerhead Cleavage | 23 |
| Sites** | |

*HCV Genotype lb was the prototype sWain
**Based on sequence alignments from HCV genotype 1a, 1b, 1c, 2a, 2b, 2c 3a, 3b, 4a, 4c, 4f, 5a, and 6a

TABLE IV
Hammerhead Ribozymes Derived from Conserved Regions of the HCV Genome

| Name | Substrate | | | Ribozyme Sequence | | |
|---|---|---|---|---|---|---|
| 5′ NCR | | | | | | |
| HCV 5-50 | CUACUGU | C | UUCACGC | GCGUGAA | CUGAUGAGGCCGUUAGGCCGAA | ACAGUAG |
| HCV 5-67 | AAAGCGU | C | UAGCCAU | AUGGCUA | CUGAUGAGGCCGUUAGGCCGAA | ACGCUUU |
| HCV 5-69 | AGCGUCU | A | GCCAUGG | CCAUGGC | CUGAUGAGGCCGUUAGGCCGAA | AGACGCU |
| HCV 5-92 | UGAGUGU | C | GUGCAGC | GCUGCAC | CUGAUGAGGCCGUUAGGCCGAA | ACACUCA |
| HCV 5-130 | GAGCCAU | A | GUGGUCU | AGACCAC | CUGAUGAGGCCGUUAGGCCGAA | AUGGCUC |
| HCV 5-136 | UAGUGGU | C | UGCGGAA | UUCCGCA | CUGAUGAGGCCGUUAGGCCGAA | ACCACUA |
| HCV 5-153 | GGUGAGU | A | CACCGGA | UCCGGUG | CUGAUGAGGCCGUUAGGCCGAA | ACUCACC |
| HCV 5-180 | ACCGGGU | C | CUUUCUU | AAGAAAG | CUGAUGAGGCCGUUAGGCCGAA | ACCCGGU |
| HCV 5-183 | GGGUCCU | U | UCUUGGA | UCCAAGA | CUGAUGAGGCCGUUAGGCCGAA | AGGACCC |
| HCV 5-184 | GGUCCUU | U | CUUGGAU | AUCCAAG | CUGAUGAGGCCGUUAGGCCGAA | AAGGACC |
| HCV 5-258 | GUUGGGU | C | GCGAAAG | CUUUCGC | CUGAUGAGGCCGUUAGGCCGAA | ACCCAAC |
| HCV 5-270 | AAGGCCU | U | GUGGUAC | GUACCAC | CUGAUGAGGCCGUUAGGCCGAA | AGGCCUU |
| HCV 5-294 | GGGUGCU | U | GCGAGUG | CACUCGC | CUGAUGAGGCCGUUAGGCCGAA | AGCACCC |
| HCV 5-313 | GGGAGGU | C | UCGUAGA | UCUACGA | CUGAUGAGGCCGUUAGGCCGAA | ACCUCCC |
| HCV 5-315 | GAGGUCU | C | GUAGACC | GGUCUAC | CUGAUGAGGCCGUUAGGCCGAA | AGACCUC |
| HCV 5-318 | GUCUCGU | A | GACCGUG | CACGGUC | CUGAUGAGGCCGUUAGGCCGAA | ACGAGAC |
| Core Region | | | | | | |
| HCV C-30 | UAAACCU | C | AAAGAAA | UUUCUUU | CUGAUGAGGCCGUUAGGCCGAA | AGGUUUA |
| HCV C-48 | CAAACGU | A | ACACCAA | UUGGUGU | CUGAUGAGGCCGUUAGGCCGAA | ACGUUUG |
| HCV C-60 | CAACCGU | C | GCCCACA | UGUGGGC | CUGAUGAGGCCGUUAGGCCGAA | ACGGUUG |
| HCV C-175 | GAGCGGU | C | ACAACCU | AGGUUGU | CUGAUGAGGCCGUUAGGCCGAA | ACCGCUC |
| HCV C-374 | GUAAGGU | C | AUCGAUA | UAUCGAU | CUGAUGAGGCCGUUAGGCCGAA | ACCUUAC |
| 3′ NCR | | | | | | |
| HCV 3-118 | UUUUUUU | U | UUUUUUU | AAAAAAA | CUGAUGAGGCCGUUAGGCCGAA | AAAAAAA |
| HCV 3-145 | GGUGGCU | C | CAUCUUA | UAAGAUG | CUGAUGAGGCCGUUAGGCCGAA | AGCCACC |
| HCV 3-149 | GCUCCAU | C | UUAGCCC | GGGCUAA | CUGAUGAGGCCGUUAGGCCGAA | AUGGAGC |
| HCV 3-151 | UCCAUCU | U | AGCCCUA | UAGGGCU | CUGAUGAGGCCGUUAGGCCGAA | AGAUGGA |
| HCV 3-152 | CCAUCUU | A | GCCCUAG | CUAGGGC | CUGAUGAGGCCGUUAGGCCGAA | AAGAUGG |
| HCV 3-158 | UAGCCCU | A | GUCACGG | CCGUGAC | CUGAUGAGGCCGUUAGGCCGAA | AGGGCUA |
| HCV 3-161 | CCCUAGU | C | ACGGCUA | UAGCCGU | CUGAUGAGGCCGUUAGGCCGAA | ACUAGGG |
| HCV 3-168 | CACGGCU | A | GCUGUGA | UCACAGC | CUGAUGAGGCCGUUAGGCCGAA | AGCCGUG |
| HCV 3-181 | GAAAGGU | C | CGUGAGC | GCUCACG | CUGAUGAGGCCGUUAGGCCGAA | ACCUUUC |

TABLE V
HCV Hammerhead Ribozyme and Target Sequence

| | | Nt. | | | | | | | | |
|---|---|---|---|---|---|---|---|---|---|---|
| No. | Name | Pos. | Hammerhead Ribozyme | | | | | Substrate | | |
| 1 | HCV-27 | 27 | UAUGGUG | CUGAUGAG | X | CGAA | AGUGUCG | CGACACU | C | CACCAUA |
| 2 | HCV-114 | 114 | GGUCCUG | CUGAUGAG | X | CGAA | AGGCUGC | GCAGCCU | C | CAGGACC |
| 3 | HCV-128 | 128 | CUCCCGG | CUGAUGAG | X | CGAA | AGGGGGG | CCCCCCU | C | CCGGGAG |
| 4 | HCV-148 | 148 | UUCCGCA | CUGAUGAG | X | CGAA | ACCACUA | UAGUGGU | C | UGCGGAA |
| 5 | HCV-165 | 165 | UCCUGUG | CUGAUGAG | X | CGAA | ACUCACC | GGUGAGU | A | CACCGGA |
| 6 | HCV-175 | 175 | UCCUGGC | CUGAUGAG | X | CGAA | AUUCCGG | CCGGAAU | U | GCCAGGA |
| 7 | HCV-199 | 199 | UUGAUCC | CUGAUGAG | X | CGAA | AGAAAGG | CCUUUCU | U | GGAUCAA |
| 8 | HCV-213 | 213 | AGGCAUU | CUGAUGAG | X | CGAA | AGCGGGU | ACCCGCU | C | AAUGCCU |
| 9 | HCV-252 | 252 | ACUCGGC | CUGAUGAG | X | CGAA | AGCAGUC | GACUGCU | A | GCCGAGU |
| 10 | HCV-260 | 260 | CCAACAC | CUGAUGAG | X | CGAA | ACUCGGC | GCCGAGU | A | GUGUUGG |
| 11 | HCV-265 | 265 | GCGACCC | CUGAUGAG | X | CGAA | ACACUAC | GUAGUGU | U | GGGUCGC |
| 12 | HCV-270 | 270 | CUUUCGC | CUGAUGAG | X | CGAA | ACCCAAC | GUUGGGU | C | GCGAAAG |
| 13 | HCV-288 | 288 | CAGGCAG | CUGAUGAG | X | CGAA | ACCACAA | UUGUGGU | A | CUGCCUG |
| 14 | HCV-298 | 298 | AGCACCC | CUGAUGAG | X | CGAA | AUCAGGC | GCCUGAU | A | GGGUGCU |
| 15 | HCV-306 | 306 | CACUCGC | CUGAUGAG | X | CGAA | AGCACCC | GGGUGCU | U | GCGAGUG |
| 16 | HCV-325 | 325 | UCUACGA | CUGAUGAG | X | CGAA | ACCUCCC | GGGAGGU | C | UCGUAGA |
| 17 | HCV-327 | 327 | GGUCUAC | CUGAUGAG | X | CGAA | AGACCUC | GAGGUCU | C | GUAGACC |
| 18 | HCV-330 | 330 | CACGGUC | CUGAUGAG | X | CGAA | ACGAGAC | GUCUCGU | A | GACCGUG |
| 19 | HCV-407 | 407 | GGAACUU | CUGAUGAG | X | CGAA | ACGUCCU | AGGACGU | C | AAGUUCC |
| 20 | HCV-412 | 412 | GCCCGGG | CUGAUGAG | X | CGAA | ACUUGAC | GUCAAGU | U | CCCGGGC |
| 21 | HCV-413 | 413 | CGCCCGG | CUGAUGAG | X | CGAA | AACUUGA | UCAAGUU | C | CCGGGCG |
| 22 | HCV-426 | 426 | ACGAUCU | CUGAUGAG | X | CGAA | ACCACCG | CGGUGGU | C | AGAUCGU |
| 23 | HCV-472 | 472 | CACACCC | CUGAUGAG | X | CGAA | ACGUGGG | CCCACGU | U | GGGUGUG |
| 24 | HCV-489 | 489 | GUCUUCC | CUGAUGAG | X | CGAA | AGUCGCG | CGCGACU | A | GGAAGAC |
| 25 | HCV-498 | 498 | CGUUCGG | CUGAUGAG | X | CGAA | AGUCUUC | GAAGACU | U | CCGAACG |
| 26 | HCV-499 | 499 | CCGUUCG | CUGAUGAG | X | CGAA | AAGUCUU | AAGACUU | C | CGAACGG |
| 27 | HCV-508 | 508 | AGGUUGC | CUGAUGAG | X | CGAA | ACCGUUC | GAACGGU | C | GCAACCU |
| 28 | HCV-534 | 534 | UUGGGGA | CUGAUGAG | X | CGAA | AGGUUGU | ACAACCU | A | UCCCCAA |
| 29 | HCV-536 | 536 | CCUUGGG | CUGAUGAG | X | CGAA | AUAGGUU | AACCUAU | C | CCCAAGG |
| 30 | HCV-546 | 546 | GGUCGGC | CUGAUGAG | X | CGAA | AGCCUUG | CAAGGCU | C | GCCGACC |
| 31 | HCV-561 | 561 | CAGGCCC | CUGAUGAG | X | CGAA | ACCCUCG | CGAGGGU | A | GGGCCUG |
| 32 | HCV-573 | 573 | CCAGGCU | CUGAUGAG | X | CGAA | AGCCCAG | CUGGGCU | C | AGCCUGG |
| 33 | HCV-583 | 583 | CCAAGGG | CUGAUGAG | X | CGAA | ACCCAGG | CCUGGGU | A | CCCUUGG |
| 34 | HCV-588 | 588 | AGGGGCC | CUGAUGAG | X | CGAA | AGGGUAC | GUACCCU | U | GGCCCCU |
| 35 | HCV-596 | 596 | UGCCAUA | CUGAUGAG | X | CGAA | AGGGGCC | GGCCCCU | C | UAUGGCA |
| 36 | HCV-598 | 598 | AUUGCCA | CUGAUGAG | X | CGAA | AGAGGGG | CCCCUCU | A | UGGCAAU |
| 37 | HCV-632 | 632 | GUGACAG | CUGAUGAG | X | CGAA | AGCCAUC | GAUGGCU | C | CUGUCAC |
| 38 | HCV-637 | 637 | GCGGGGU | CUGAUGAG | X | CGAA | ACAGGAG | CUCCUGU | C | ACCCCGC |
| 39 | HCV-649 | 649 | AGGCCGG | CUGAUGAG | X | CGAA | AGCCGCG | CGCGGCU | C | CCGGCCU |
| 40 | HCV-657 | 657 | CCCCAAC | CUGAUGAG | X | CGAA | AGGCCGG | CCGGCCU | A | GUUGGGG |
| 41 | HCV-660 | 660 | GGGCCCC | CUGAUGAG | X | CGAA | ACUAGGC | GCCUAGU | U | GGGGCCC |
| 42 | HCV-696 | 696 | UUACCCA | CUGAUGAG | X | CGAA | AUUGCGC | GCGCAAU | C | UGGGUAA |
| 43 | HCV-707 | 707 | UAUCGAU | CUGAUGAG | X | CGAA | ACCUUAC | GUAAGGU | C | AUCGAUA |
| 44 | HCV-710 | 710 | GGGUAUC | CUGAUGAG | X | CGAA | AUGACCU | AGGUCAU | C | GAUACCC |
| 45 | HCV-714 | 714 | GUGAGGG | CUGAUGAG | X | CGAA | AUCCAUG | CAUCGAU | A | CCCUCAC |
| 46 | HCV-730 | 730 | GUCGGCG | CUGAUGAG | X | CGAA | AGCCGCA | UGCGGCU | U | CGCCGAC |
| 47 | HCV-731 | 731 | GGUCGGC | CUGAUGAG | X | CGAA | AAGCCGC | GCGGCUU | C | GCCGACC |
| 48 | HCV-748 | 748 | CGGAAUG | CUGAUGAG | X | CGAA | ACCCCAU | AUGGGGU | A | CAUUCCG |
| 49 | HCV-752 | 752 | CGAGCGG | CUGAUGAG | X | CGAA | AUGUACC | GGUACAU | U | CCGCUCG |
| 50 | HCV-753 | 753 | ACGAGCG | CUGAUGAG | X | CGAA | AAUGUAC | GUACAUU | C | CGCUCGU |
| 51 | HCV-758 | 758 | CGCCGAC | CUGAUGAG | X | CGAA | AGCGGAA | UUCCGCU | C | GUCGGCG |
| 52 | HCV-761 | 761 | GGGCGCC | CUGAUGAG | X | CGAA | ACGAGCG | CGCUCGU | C | GGCGCCC |
| 53 | HCV-773 | 773 | CGCCCCC | CUGAUGAG | X | CGAA | AGGGGGG | CCCCCCU | A | GGGGGCG |
| 54 | HCV-806 | 806 | GAACCCG | CUGAUGAG | X | CGAA | ACACCAU | AUGGUGU | C | CGGGUUC |
| 55 | HCV-812 | 812 | CCUCCAG | CUGAUGAG | X | CGAA | ACCCGGA | UCCGGGU | U | CUGGAGG |
| 56 | HCV-813 | 813 | UCCUCCA | CUGAUGAG | X | CGAA | AACCCGG | CCGGGUU | C | UGGAGGA |
| 57 | HCV-832 | 832 | UGUUGCG | CUGAUGAG | X | CGAA | AGUUCAC | GUGAACU | A | CGCAACA |
| 58 | HCV-847 | 847 | ACCGGGC | CUGAUGAG | X | CGAA | AGUUCCC | GGGAACU | U | GCCCGGU |
| 59 | HCV-855 | 855 | AAAGAGC | CUGAUGAG | X | CGAA | ACCGGGC | GCCCGGU | U | GCUCUUU |
| 60 | HCV-859 | 859 | AGAGAAA | CUGAUGAG | X | CGAA | AGCAACC | GGUUGCU | C | UUUCUCU |
| 61 | HCV-982 | 982 | UGCCUCA | CUGAUGAG | X | CGAA | ACACAAU | AUUGUGU | A | UGAGGCA |
| 62 | HCV-1001 | 1001 | UAUGCAU | CUGAUGAG | X | CGAA | AUCAUGC | GCAUGAU | C | AUGCAUA |
| 63 | HCV-1022 | 1022 | CGCAGGG | CUGAUGAG | X | CGAA | ACGCACC | GGUGCGU | A | CCCUGCG |
| 64 | HCV-1031 | 1031 | UCUCCCG | CUGAUGAG | X | CGAA | ACGCAGG | CCUGCGU | U | CGGGAGA |
| 65 | HCV-1032 | 1032 | UUCUCCC | CUGAUGAG | X | CGAA | AACGCAG | CUGCGUU | C | GGGAGAA |
| 66 | HCV-1048 | 1048 | ACAACGG | CUGAUGAG | X | CGAA | AGGCGUU | AACGCCU | C | CCGUUGU |
| 67 | HCV-1053 | 1053 | ACCCAAC | CUGAUGAG | X | CGAA | ACGGGAG | CUCCCGU | U | GUUGGGU |
| 68 | HCV-1056 | 1056 | GCUACCC | CUGAUGAG | X | CGAA | ACAACGG | CCGUUGU | U | GGGUAGC |
| 69 | HCV-1061 | 1061 | UGAGCGC | CUGAUGAG | X | CGAA | ACCCAAC | GUUGGGU | A | GCGCUCA |
| 70 | HCV-1127 | 1127 | GCAAGUC | CUGAUGAG | X | CGAA | ACGUGGC | GCCACGU | C | GACUUGC |
| 71 | HCV-1132 | 1132 | AACGAGC | CUGAUGAG | X | CGAA | AGUCGAC | GUCGACU | U | GCUCGUU |
| 72 | HCV-1136 | 1136 | CCCCAAC | CUGAUGAG | X | CGAA | AGCAAGU | ACUUGCU | C | GUUGGGG |
| 73 | HCV-1139 | 1139 | CCGCCCC | CUGAUGAG | X | CGAA | ACGAGCA | UGCUCGU | U | GGGGCGG |
| 74 | HCV-1153 | 1153 | GGAACAG | CUGAUGAG | X | CGAA | AAGCGGC | GCCGCUU | U | CUGUUCC |
| 75 | HCV-1154 | 1154 | CGGAACA | CUGAUGAG | X | CGAA | AAAGCGG | CCGCUUU | C | UGUUCCG |
| 76 | HCV-1158 | 1158 | AUGGCGG | CUGAUGAG | X | CGAA | ACAGAAA | UUUCUGU | U | CCGCCAU |
| 77 | HCV-1159 | 1159 | CAUGGCG | CUGAUGAG | X | CGAA | AACAGAA | UUCUGUU | C | CGCCAUG |
| 78 | HCV-1168 | 1168 | CCCCACG | CUGAUGAG | X | CGAA | ACAUGGC | GCCAUGU | A | CGUGGGG |
| 79 | HCV-1189 | 1189 | GAAAACG | CUGAUGAG | X | CGAA | AUCCGCA | UGCGGAU | C | CGUUUUC |
| 80 | HCV-1193 | 1193 | CGAGGAA | CUGAUGAG | X | CGAA | ACGGAUC | GAUCCGU | U | UUCCUCG |
| 81 | HCV-1194 | 1194 | ACGAGGA | CUGAUGAG | X | CGAA | AACGGAU | AUCCGUU | U | UCCUCGU |
| 82 | HCV-1195 | 1195 | GACGAGG | CUGAUGAG | X | CGAA | AAACGGA | UCCGUUU | U | CCUCGUC |
| 83 | HCV-1196 | 1196 | AGACGAG | CUGAUGAG | X | CGAA | AAAACGG | CCGUUUU | C | CUCGUCU |
| 84 | HCV-1280 | 1280 | GACCUGA | CUGAUGAG | X | CGAA | ACAUGGC | GCCAUGU | A | UCAGGUC |
| 85 | HCV-1282 | 1282 | GUGACCU | CUGAUGAG | X | CGAA | AUACAUG | CAUGUAU | C | AGGUCAC |
| 86 | HCV-1287 | 1287 | AUGCGGU | CUGAUGAG | X | CGAA | ACCUGAU | AUCAGGU | C | ACCGCAU |
| 87 | HCV-1373 | 1373 | UAUCCAC | CUGAUGAG | X | CGAA | ACAGCUU | AAGCUGU | C | GUGGAUA |
| 88 | HCV-1380 | 1380 | GCCACCA | CUGAUGAG | X | CGAA | AUCCACG | CGUGGAU | A | UGGUGGC |
| 89 | HCV-1406 | 1406 | CCGCUAG | CUGAUGAG | X | CGAA | ACUCCCC | GGGGAGU | C | CUAGCGG |
| 90 | HCV-1409 | 1409 | GGCCCGC | CUGAUGAG | X | CGAA | AGGACUC | GAGUCCU | A | GCGGGCC |
| 91 | HCV-1418 | 1418 | AGUAGGC | CUGAUGAG | X | CGAA | AGGCCCG | CGGGCCU | U | GCCUACU |
| 92 | HCV-1423 | 1423 | GGAAUAG | CUGAUGAG | X | CGAA | AGGCAAG | CUUGCCU | A | CUAUUCC |
| 93 | HCV-1426 | 1426 | CAUGGAA | CUGAUGAG | X | CGAA | AGUAGGC | GCCUACU | A | UUCCAUG |
| 94 | HCV-1428 | 1428 | ACCAUGG | CUGAUGAG | X | CGAA | AUAGUAG | CUACUAU | U | CCAUGGU |
| 95 | HCV-1429 | 1429 | CACCAUG | CUGAUGAG | X | CGAA | AAUAGUA | UACUAUU | C | CAUGGUG |
| 96 | HCV-1727 | 1727 | ACUUGUC | CUGAUGAG | X | CGAA | AUGGAGC | GCUCCAU | C | GACAAGU |
| 97 | HCV-1735 | 1735 | CUGAGCG | CUGAUGAG | X | CGAA | ACUUGUC | GACAAGU | U | CGCUCAG |
| 98 | HCV-1736 | 1736 | CCUGAGC | CUGAUGAG | X | CGAA | AACUUGU | ACAAGUU | C | GCUCAGG |
| 99 | HCV-1740 | 1740 | CAUCCCU | CUGAUGAG | X | CGAA | AGCGAAC | GUUCGCU | C | AGGGAUG |
| 100 | HCV-1757 | 1757 | UAUAGGU | CUGAUGAG | X | CGAA | AUGGGGC | GCCCCAU | C | ACCUAUA |
| 101 | HCV-1762 | 1762 | CUCGGUA | CUGAUGAG | X | CGAA | AGGUGAU | AUCACCU | A | UACCGAG |
| 102 | HCV-1795 | 1795 | CCAGCAG | CUGAUGAG | X | CGAA | AAGGCCU | AGGCCUU | A | CUGCUGG |
| 103 | HCV-1806 | 1806 | GGUGCGU | CUGAUGAG | X | CGAA | AUGCCAG | CUGGCAU | U | ACGCACC |
| 104 | HCV-1807 | 1807 | AGGUGCG | CUGAUGAG | X | CGAA | AAUGCCA | UGGCAUU | A | CGCACCU |
| 105 | HCV-1815 | 1815 | CACUGCC | CUGAUGAG | X | CGAA | AGGUGCG | CGCACCU | C | GGCAGUG |
| 106 | HCV-1827 | 1827 | GGUACGA | CUGAUGAG | X | CGAA | ACCACAC | GUGUGGU | A | UCGUACC |
| 107 | HCV-1829 | 1829 | CAGGUAC | CUGAUGAG | X | CGAA | AUACCAC | GUGGUAU | C | GUACCUG |
| 108 | HCV-1832 | 1832 | ACGCAGG | CUGAUGAG | X | CGAA | ACGAUAC | GUAUCGU | A | CCUGCGU |
| 109 | HCV-1840 | 1840 | CACCUGC | CUGAUGAG | X | CGAA | ACGCAGG | CCUGCGU | C | GCAGGUG |
| 110 | HCV-1854 | 1854 | UACACUG | CUGAUGAG | X | CGAA | ACCACAC | GUGUGGU | C | CAGUGUA |
| 111 | HCV-1883 | 1883 | CCACUAC | CUGAUGAG | X | CGAA | ACAGGGC | GCCCUGU | U | GUAGUGG |
| 112 | HCV-1886 | 1886 | UCCCCAC | CUGAUGAG | X | CGAA | ACAACAG | CUGUUGU | A | GUGGGGA |
| 113 | HCV-1902 | 1902 | CCGGACC | CUGAUGAG | X | CGAA | AUCGGUC | GACCGAU | C | GGUCCGG |
| 114 | HCV-1906 | 1906 | GGCACCG | CUGAUGAG | X | CGAA | ACCGAUC | GAUCGGU | C | CGGUGCC |
| 115 | HCV-1917 | 1917 | UUAUACG | CUGAUGAG | X | CGAA | AGGGGCA | UGCCCCU | A | CGUAUAA |
| 116 | HCV-1921 | 1921 | CCAGUUA | CUGAUGAG | X | CGAA | ACGUAGG | CCUACGU | A | UAACUGG |
| 117 | HCV-1923 | 1923 | CCCCAGU | CUGAUGAG | X | CGAA | AUACGUA | UACGUAU | A | ACUGGGG |
| 118 | HCV-1990 | 1990 | ACAGCCA | CUGAUGAG | X | CGAA | ACCAGUU | AACUGGU | U | UGGCUGU |
| 119 | HCV-1991 | 1991 | UACAGCC | CUGAUGAG | X | CGAA | AACCAGU | ACUGGUU | U | GGCUGUA |
| 120 | HCV-1998 | 1998 | AUCCAUG | CUGAUGAG | X | CGAA | ACAGCCA | UGGCUGU | A | CAUGGAU |
| 121 | HCV-2043 | 2043 | UUGCACG | CUGAUGAG | X | CGAA | AGGGCCC | GGGCCCU | C | CGUGCAA |
| 122 | HCV-2054 | 2054 | CCCCCCC | CUGAUGAG | X | CGAA | AUGUUGC | GCAACAU | C | GGGGGGG |
| 123 | HCV-2063 | 2083 | GGUUGCC | CUGAUGAG | X | CGAA | ACCCCCC | GGGGGGU | C | GGCAACC |
| 124 | HCV-2072 | 2072 | UCAAGGU | CUGAUGAG | X | CGAA | AGGUUGC | GCAACCU | C | ACCUUGA |
| 125 | HCV-2077 | 2077 | GCAGGUC | CUGAUGAG | X | CGAA | AGGUGAG | CUCACCU | U | GACCUGC |
| 126 | HCV-2121 | 2121 | UUUGUGU | CUGAUGAG | X | CGAA | AGUGGCC | GGCCACU | U | ACACAAA |
| 127 | HCV-2122 | 2122 | UUUUGUG | CUGAUGAG | X | CGAA | AAGUGGC | GCCACUU | A | CACAAAA |
| 128 | HCV-2137 | 2137 | UGGCCCC | CUGAUGAG | X | CGAA | AGCCACA | UGUGGCU | C | GGGGCCA |
| 129 | HCV-2149 | 2149 | AGGUGUU | CUGAUGAG | X | CGAA | ACCAUGG | CCAUGGU | U | AACACCU |
| 130 | HCV-2150 | 2150 | UAGGUGU | CUGAUGAG | X | CGAA | AACCAUG | CAUGGUU | A | ACACCUA |
| 131 | HCV-2219 | 2219 | CCUUAAA | CUGAUGAG | X | CGAA | AUGGUAA | UUACCAU | C | UUUAAGG |
| 132 | HCV-2221 | 2221 | AACCUUA | CUGAUGAG | X | CGAA | AGAUGGU | ACCAUCU | U | UAAGGUU |
| 133 | HCV-2261 | 2261 | CAGCACU | CUGAUGAG | X | CGAA | AGCCUGU | ACAGGCU | U | AGUGCUG |
| 134 | HCV-2262 | 2262 | GCAGCAC | CUGAUGAG | X | CGAA | AAGCCUG | CAGGCUU | A | GUGCUGC |
| 135 | HCV-2295 | 2295 | AGGUCGC | CUGAUGAG | X | CGAA | ACGCUCU | AGAGCGU | U | GCGACCU |
| 136 | HCV-2320 | 2320 | GAGCUCC | CUGAUGAG | X | CGAA | AUCUGUC | GACAGAU | C | GGAGCUC |
| 137 | HCV-2327 | 2327 | GCGGGCU | CUGAUGAG | X | CGAA | AGCUCCG | CGGAGCU | C | AGCCCGC |
| 138 | HCV-2344 | 2344 | UGUCGUG | CUGAUGAG | X | CGAA | ACAGCAG | CUGCUGU | C | CACGACA |
| 139 | HCV-2417 | 2417 | UCUGAUG | CUGAUGAG | X | CGAA | AGGUGGA | UCCACCU | C | CAUCAGA |
| 140 | HCV-2421 | 2421 | AUGUUCU | CUGAUGAG | X | CGAA | AUGGAGG | CCUCCAU | C | AGAACAU |
| 141 | HCV-2429 | 2429 | CGUCCAC | CUGAUGAG | X | CGAA | AUGUUCU | AGAACAU | C | GUGGACG |
| 142 | HCV-2534 | 2534 | AGGCACA | CUGAUGAG | X | CGAA | ACGCGCG | CGCGCGU | C | UGUGCCU |
| 143 | HCV-2585 | 2585 | GGUUCUC | CUGAUGAG | X | CGAA | AGGGCGG | CCGCCCU | A | GAGAACC |
| 144 | HCV-2600 | 2600 | CGUUGAG | CUGAUGAG | X | CGAA | ACCACCA | UGGUGGU | C | CUCAACG |
| 145 | HCV-2603 | 2603 | CCGCGUU | CUGAUGAG | X | CGAA | AGGACCA | UGGUCCU | C | AACGCGG |
| 146 | HCV-2671 | 2671 | CUUGAUG | CUGAUGAG | X | CGAA | ACCAGGC | GCCUGGU | A | CAUCAAG |
| 147 | HCV-2675 | 2675 | UGCCCUU | CUGAUGAG | X | CGAA | AUGUACC | GGUACAU | C | AAGGGCA |
| 148 | HCV-2690 | 2690 | CCCCAGG | CUGAUGAG | X | CGAA | ACCAGCC | GGCUGGU | C | CCUGGGG |
| 149 | HCV-2704 | 2704 | CAGAGCA | CUGAUGAG | X | CGAA | AUGCCGC | GCGGCAU | A | UGCUCUG |
| 150 | HCV-2709 | 2709 | CCGUACA | CUGAUGAG | X | CGAA | AGCAUAU | AUAUGCU | C | UGUACGG |
| 151 | HCV-2713 | 2713 | CACGCCG | CUGAUGAG | X | CGAA | ACAGAGC | GCUCUGU | A | CGGCGUG |
| 152 | HCV-2738 | 2738 | CCAGCAG | CUGAUGAG | X | CGAA | ACCAGGA | UCCUGCU | C | CUGCUGG |
| 153 | HCV-2763 | 2763 | AUGGCGU | CUGAUGAG | X | CGAA | AGCCCGU | ACGGGCU | U | ACGCCAU |
| 154 | HCV-2764 | 2764 | CAUGGCG | CUGAUGAG | X | CGAA | AAGCCCG | CGGGCUU | A | CGCCAUG |
| 155 | HCV-2878 | 2878 | GUAUUGU | CUGAUGAG | X | CGAA | ACCACCA | UGGUGGU | U | ACAAUAC |
| 156 | HCV-2879 | 2879 | AGUAUUG | CUGAUGAG | X | CGAA | AACCACC | GGUGGUU | A | CAAUACU |
| 157 | HCV-2884 | 2884 | GAUAAAG | CUGAUGAG | X | CGAA | AUUGUAA | UUACAAU | A | CUUUAUC |
| 158 | HCV-2887 | 2887 | GGUGAUA | CUGAUGAG | X | CGAA | AGUAUUG | CAAUACU | U | UAUCACC |
| 159 | HCV-2888 | 2888 | UGGUGAU | CUGAUGAG | X | CGAA | AAGUAUU | AAUACUU | U | AUCACCA |
| 160 | HCV-2910 | 2910 | ACGCACA | CUGAUGAG | X | CGAA | AUGCGCC | GGCGCAU | U | UGUGCGU |
| 161 | HCV-2911 | 2911 | CACGCAC | CUGAUGAG | X | CGAA | AAUGCGC | GCGCAUU | U | GUGCGUG |
| 162 | HCV-2924 | 2924 | GAGGGGG | CUGAUGAG | X | CGAA | ACCCACA | UGUGGGU | C | CCCCCUC |
| 163 | HCV-2931 | 2931 | ACAUUGA | CUGAUGAG | X | CGAA | AGGGGGG | CCCCCCU | C | UCAAUGU |
| 164 | HCV-2933 | 2933 | GGACAUU | CUGAUGAG | X | CGAA | AGAGGGG | CCCCUCU | C | AAUGUCC |
| 165 | HCV-2939 | 2939 | CCCCCCG | CUGAUGAG | X | CGAA | ACAUUGA | UCAAUGU | C | CGGGGGG |
| 166 | HCV-2958 | 2958 | AGGAUGA | CUGAUGAG | X | CGAA | AGCAUCG | CGAUGCU | A | UCAUCCU |
| 167 | HCV-2960 | 2960 | GGAGGAU | CUGAUGAG | X | CGAA | AUAGCAU | AUGCUAU | C | AUCCUCC |
| 168 | HCV-2963 | 2963 | UGAGGAG | CUGAUGAG | X | CGAA | AUGAUAG | CUAUCAU | C | CUCCUCA |
| 169 | HCV-2966 | 2966 | AUGUGAG | CUGAUGAG | X | CGAA | AGGAUGA | UCAUCCU | C | CUCACAU |
| 170 | HCV-2969 | 2969 | CACAUGU | CUGAUGAG | X | CGAA | AGGAGGA | UCCUCCU | C | ACAUGUG |
| 171 | HCV-3059 | 3059 | UCGCAGU | CUGAUGAG | X | CGAA | AUGGCAG | CUGCCAU | A | ACUGCGA |
| 172 | HCV-3138 | 3138 | UGGACGU | CUGAUGAG | X | CGAA | AUGGCCU | AGGCCAU | U | ACGUCCA |
| 173 | HCV-3139 | 3139 | UUGGACG | CUGAUGAG | X | CGAA | AAUGGCC | GGCCAUU | A | CGUCCAA |
| 174 | HCV-3143 | 3143 | CCAUUUG | CUGAUGAG | X | CGAA | ACGUAAU | AUUACGU | C | CAAAUGG |
| 175 | HCV-3154 | 3154 | CUUCAUG | CUGAUGAG | X | CGAA | AGGCCAU | AUGGCCU | U | CAUGAAG |
| 176 | HCV-3155 | 3155 | GCUUCAU | CUGAUGAG | X | CGAA | AAGGCCA | UGGCCUU | C | AUGAAGC |
| 177 | HCV-3209 | 3209 | AAUCCUG | CUGAUGAG | X | CGAA | AGCGGGG | CCCCGCU | A | CAGGAUU |
| 178 | HCV-3216 | 3216 | UGGGCCC | CUGAUGAG | X | CGAA | AUCCUGU | ACAGGAU | U | GGGCCCA |
| 179 | HCV-3233 | 3233 | GGUCUCG | CUGAUGAG | X | CGAA | AGGCCCG | CGGGCCU | A | CGAGACC |
| 180 | HCV-3242 | 3242 | CCACCGC | CUGAUGAG | X | CGAA | AGGUCUC | GAGACCU | U | GCGGUGG |
| 181 | HCV-3263 | 3263 | AGAAGAC | CUGAUGAG | X | CGAA | ACGGGCU | AGCCCGU | C | GUCUUCU |
| 182 | HCV-3266 | 3266 | CAGAGAA | CUGAUGAG | X | CGAA | ACGACGG | CCGUCGU | C | UUCUCUG |
| 183 | HCV-3268 | 3268 | GUCAGAG | CUGAUGAG | X | CGAA | AGACGAC | GUCGUCU | U | CUCUGAC |
| 184 | HCV-3290 | 3290 | AGGUGAU | CUGAUGAG | X | CGAA | AUCUUGG | CCAAGAU | C | AUCACCU |
| 185 | HCV-3293 | 3293 | CCCAGGU | CUGAUGAG | X | CGAA | AUGAUCU | AGAUCAU | C | ACCUGGG |
| 186 | HCV-3329 | 3329 | CCAAGAU | CUGAUGAG | X | CGAA | AUGUCCC | GGGACAU | C | AUCUUGG |
| 187 | HCV-3332 | 3332 | GUCCCAA | CUGAUGAG | X | CGAA | AUGAUGU | ACAUCAU | C | UUGGGAC |
| 188 | HCV-3334 | 3334 | CAGUCCC | CUGAUGAG | X | CGAA | AGAUGAU | AUCAUCU | U | GGGACUG |
| 189 | HCV-3347 | 3347 | GGGCGGA | CUGAUGAG | X | CGAA | ACGGGCA | UGCCCGU | C | UCCGCCC |
| 190 | HCV-3349 | 3349 | UCGGGCG | CUGAUGAG | X | CGAA | ACACGGG | CCCGUCU | C | CGCCCGA |
| 191 | HCV-3371 | 3371 | CCAGAAG | CUGAUGAG | X | CGAA | AUCUCCC | GGGAGAU | A | CUUCUGG |
| 192 | HCV-3416 | 3416 | GGGCAAG | CUGAUGAG | X | CGAA | AGUCGCC | GGCGACU | C | CUUGCCC |
| 193 | HCV-3419 | 3419 | UGGGGGC | CUGAUGAG | X | CGAA | AGGAGUC | GACUCCU | U | GCCCCCA |
| 194 | HCV-3428 | 3428 | AGGCCGU | CUGAUGAG | X | CGAA | AUGGGGG | CCCCCAU | C | ACGGCCU |
| 195 | HCV-3482 | 3482 | GGCCUGU | CUGAUGAG | X | CGAA | AGUCUAG | CUAGCCU | C | ACAGGCC |
| 196 | HCV-3518 | 3518 | CCACUUG | CUGAUGAG | X | CGAA | ACCUCCC | GGGAGGU | U | CAAGUGG |
| 197 | HCV-3519 | 3519 | ACCACUU | CUGAUGAG | X | CGAA | AACCUCC | GGAGGUU | C | AAGUGGU |
| 198 | HCV-3527 | 3527 | CGGUGGA | CUGAUGAG | X | CGAA | ACCACUU | AAGUGGU | U | UCCACCG |
| 199 | HCV-3528 | 3528 | GCGGUGG | CUGAUGAG | X | CGAA | AACCACU | AGUGGUU | U | CCACCGC |
| 200 | HCV-3529 | 3529 | UGCGGUG | CUGAUGAG | X | CGAA | AAACCAC | GUGGUUU | C | CACCGCA |
| 201 | HCV-3576 | 3576 | ACGGUCC | CUGAUGAG | X | CGAA | ACACACA | UGUGUGU | U | GGACCGU |
| 202 | HCV-3601 | 3601 | GGUCUUU | CUGAUGAG | X | CGAA | AGCCGGC | GCCGGCU | C | AAAGACC |
| 203 | HCV-3611 | 3611 | GGCCGGC | CUGAUGAG | X | CGAA | AGGGUCU | AGACCCU | A | GCCGGCC |
| 204 | HCV-3684 | 3684 | GCCCCGG | CUGAUGAG | X | CGAA | AGGCGCA | UGCGCCU | C | CCGGGGC |
| 205 | HCV-3696 | 3696 | GUAAGGG | CUGAUGAG | X | CGAA | ACGCGCC | GGCGCGU | U | CCCUUAC |
| 206 | HCV-3697 | 3697 | UGUAAGG | CUGAUGAG | X | CGAA | AACGCGC | GCGCGUU | C | CCUUACA |
| 207 | HCV-3701 | 3701 | AUGGUGU | CUGAUGAG | X | CGAA | AGGGAAC | GUUCCCU | U | ACACCAU |
| 208 | HCV-3702 | 3702 | CAUGGUG | CUGAUGAG | X | CGAA | AAGGGAA | UUCCCUU | A | CACCAUG |
| 209 | HCV-3724 | 3724 | GAGGUCC | CUGAUGAG | X | CGAA | AGCUACC | GGUAGCU | C | GGACCUC |
| 210 | HCV-3731 | 3731 | CCAGAUA | CUGAUGAG | X | CGAA | AGGUCCG | CGGACCU | C | UAUCUGG |
| 211 | HCV-3733 | 3733 | GACCAGA | CUGAUGAG | X | CGAA | AGAGGUC | GACCUCU | A | UCUGGUC |
| 212 | HCV-3735 | 3735 | GUGACCA | CUGAUGAG | X | CGAA | AUAGAGG | CCUCUAU | C | UGGUCAC |
| 213 | HCV-3740 | 3740 | GUCUCGU | CUGAUGAG | X | CGAA | ACCAGAU | AUCUGGU | C | ACGAGAC |
| 214 | HCV-3761 | 3761 | GCACCGG | CUGAUGAG | X | CGAA | AUGACGU | ACGUCAU | U | CCGGUGC |
| 215 | HCV-3762 | 3762 | CGCACCG | CUGAUGAG | X | CGAA | AAUGACG | CGUCAUU | C | CGGUGCG |
| 216 | HCV-3786 | 3786 | CUCCCCC | CUGAUGAG | X | CGAA | ACCGUCA | UGACGGU | C | GGGGGAG |
| 217 | HCV-3797 | 3797 | GGGACAG | CUGAUGAG | X | CGAA | AGGCUCC | GGAGCCU | A | CUGUCCC |
| 218 | HCV-3802 | 3802 | UCUGGGG | CUGAUGAG | X | CGAA | ACAGUAG | CUACUGU | C | CCCCAGA |
| 219 | HCV-3835 | 3835 | GCCACCC | CUGAUGAG | X | CGAA | AAGAGCC | GGCUCUU | C | GGGUGGC |
| 220 | HCV-3851 | 3851 | AAGGGCA | CUGAUGAG | X | CGAA | AGCAGUG | CACUGCU | C | UGCCCUU |
| 221 | HCV-3858 | 3858 | UGCCCCG | CUGAUGAG | X | CGAA | AGGGCAG | CUGCCCU | U | CGGGGCA |
| 222 | HCV-3859 | 3859 | GUGCCCC | CUGAUGAG | X | CGAA | AAGGGCA | UGCCCUU | C | GGGGCAC |
| 223 | HCV-3872 | 3872 | AGAUGCC | CUGAUGAG | X | CGAA | ACAGCGU | ACGCUGU | A | GGCAUCU |
| 224 | HCV-3878 | 3878 | CCCGGAA | CUGAUGAG | X | CGAA | AUGCCUA | UAGGCAU | C | UUCCGGG |
| 225 | HCV-3880 | 3880 | AGCCCGG | CUGAUGAG | X | CGAA | AGAUGCC | GGCAUCU | U | CCGGGCU |
| 226 | HCV-3881 | 3881 | CAGCCCG | CUGAUGAG | X | CGAA | AAGAUGC | GCAUCUU | C | CGGGCUG |
| 227 | HCV-3908 | 3908 | CCUUCGC | CUGAUGAG | X | CGAA | ACCCCCC | GGGGGGU | U | GCGAAGG |
| 228 | HCV-4056 | 4056 | GGCACUU | CUGAUGAG | X | CGAA | AGUGCUC | GAGCACU | A | AAGUGCC |
| 229 | HCV-4072 | 4072 | GGCUGCG | CUGAUGAG | X | CGAA | ACGCAGC | GCUGCGU | A | CGCAGCC |
| 230 | HCV-4087 | 4087 | UACCUUG | CUGAUGAG | X | CGAA | ACCCUUG | CAAGGGU | A | CAAGGUA |
| 231 | HCV-4115 | 4115 | UGGCGGC | CUGAUGAG | X | CGAA | ACAGAUG | CAUCUGU | U | GCCGCCA |
| 232 | HCV-4175 | 4175 | CAGUUCU | CUGAUGAG | X | CGAA | AUGUUGG | CCAACAU | C | AGAACUG |
| 233 | HCV-4187 | 4187 | UGGUCCU | CUGAUGAG | X | CGAA | ACCCCAG | CUGGGGU | A | AGGACCA |
| 234 | HCV-4228 | 4228 | CUUACCA | CUGAUGAG | X | CGAA | AGGUGGA | UCCACCU | A | UGGUAAG |
| 235 | HCV-4233 | 4233 | AGGAACU | CUGAUGAG | X | CGAA | ACCAUAG | CUAUGGU | A | AGUUCCU |
| 236 | HCV-4237 | 4237 | GGCAAGG | CUGAUGAG | X | CGAA | ACUUACC | GGUAAGU | U | CCUUGCC |
| 237 | HCV-4238 | 4238 | CGGCAAG | CUGAUGAG | X | CGAA | AACUUAC | GUAAGUU | C | CUUGCCG |
| 238 | HCV-4241 | 4241 | CGUCGGC | CUGAUGAG | X | CGAA | AGGAACU | AGUUCCU | U | GCCGACG |
| 239 | HCV-4280 | 4280 | CACAUAU | CUGAUGAG | X | CGAA | AUGAUAU | AUAUCAU | A | AUAUGUG |
| 240 | HCV-4283 | 4283 | CAUCACA | CUGAUGAG | X | CGAA | AUUAUGA | UCAUAAU | A | UGUGAUG |
| 241 | HCV-4337 | 4337 | GGUCCAG | CUGAUGAG | X | CGAA | ACUGUGC | GCACAGU | C | CUGGACC |
| 242 | HCV-4370 | 4370 | GCACGAC | CUGAUGAG | X | CGAA | AGCCGCG | CGCGGCU | C | GUCGUGC |
| 243 | HCV-4373 | 4373 | CGAGCAC | CUGAUGAG | X | CGAA | ACGAGCC | GGCUCGU | C | GUGCUCG |
| 244 | HCV-4379 | 4379 | CGGUGGC | CUGAUGAG | X | CGAA | AGCACGA | UCGUGCU | C | GCCACCG |
| 245 | HCV-4425 | 4425 | UCCUCAA | CUGAUGAG | X | CGAA | AUUUGGG | CCCAAAU | A | UUGAGGA |
| 246 | HCV-4444 | 4444 | AGUGUUG | CUGAUGAG | X | CGAA | ACAGAGC | GCUCUGU | C | CAACACU |
| 247 | HCV-4460 | 4460 | AGAAGGG | CUGAUGAG | X | CGAA | AUCUCUC | GAGAGAU | C | CCCUUCU |
| 248 | HCV-4481 | 4481 | CGAGGGG | CUGAUGAG | X | CGAA | AUGGCCU | AGGCCAU | C | CCCCUCG |
| 249 | HCV-4487 | 4487 | UGGCCUC | CUGAUGAG | X | CGAA | AGGGGGA | UCCCCCU | C | GAGGCCA |
| 250 | HCV-4496 | 4496 | CCCCCUU | CUGAUGAG | X | CGAA | AUGGCCU | AGGCCAU | C | AAGGGGG |
| 251 | HCV-4528 | 4528 | CUUCUUG | CUGAUGAG | X | CGAA | AGUGGCA | UGCCACU | C | CAAGAAG |
| 252 | HCV-4577 | 4577 | CGGCAUU | CUGAUGAG | X | CGAA | AUUCCGA | UCGGAAU | C | AAUGCCG |
| 253 | HCV-4586 | 4586 | AAUACGC | CUGAUGAG | X | CGAA | ACGGCAU | AUGCCGU | A | GCGUAUU |
| 254 | HCV-4591 | 4591 | CCGGUAA | CUGAUGAG | X | CGAA | ACGCUAC | GUAGCGU | A | UUACCGG |
| 255 | HCV-4593 | 4593 | CCCCGGU | CUGAUGAG | X | CGAA | AUACGCU | AGCGUAU | U | ACCGGGG |
| 256 | HCV-4594 | 4594 | ACCCCGG | CUGAUGAG | X | CGAA | AAUACGC | GCGUAUU | A | CCGGGGU |
| 257 | HCV-4616 | 4616 | UCGGUAU | CUGAUGAG | X | CGAA | ACGGACA | UGUCCGU | C | AUACCGA |
| 258 | HCV-4619 | 4619 | UAGUCGG | CUGAUGAG | X | CGAA | AUGACGG | CCGUCAU | A | CCGACUA |
| 259 | HCV-4626 | 4626 | UCUCCGC | CUGAUGAG | X | CGAA | AGUCGGU | ACCGACU | A | GCGGAGA |
| 260 | HCV-4672 | 4672 | ACCGGUG | CUGAUGAG | X | CGAA | AGCCCGU | ACGGGCU | A | CACCGGU |
| 261 | HCV-4697 | 4697 | UGCAGUC | CUGAUGAG | X | CGAA | AUCACCG | CGGUGAU | C | GACUGCA |
| 262 | HCV-4789 | 4789 | UGAGCGC | CUGAUGAG | X | CGAA | ACACCGC | GCGGUGU | C | GCGCUCA |
| 263 | HCV-4795 | 4795 | CCGUUGU | CUGAUGAG | X | CGAA | AGCGCGA | UCGCGCU | C | ACAACGG |
| 264 | HCV-4920 | 4920 | UCAUACC | CUGAUGAG | X | CGAA | AGCACAG | CUGUGCU | U | GGUAUGA |
| 265 | HCV-4924 | 4924 | GAGCUCA | CUGAUGAG | X | CGAA | ACCAAGC | GCUUGGU | A | UGAGCUC |
| 266 | HCV-4931 | 4931 | CGGGCGU | CUGAUGAG | X | CGAA | AGCUCAU | AUGAGCU | C | ACGCCCG |
| 267 | HCV-4947 | 4947 | CUGACUG | CUGAUGAG | X | CGAA | AGUCUCA | UGAGACU | A | CAGUCAG |
| 268 | HCV-4952 | 4952 | GCAACCU | CUGAUGAG | X | CGAA | ACUGUAG | CUACAGU | C | AGGUUGC |
| 269 | HCV-4957 | 4957 | AGCCCGC | CUGAUGAG | X | CGAA | ACCUGAC | GUCAGGU | U | GCGGGCU |
| 270 | HCV-4965 | 4965 | UUCAGGU | CUGAUGAG | X | CGAA | AGCCCGC | GCGGGCU | U | ACCUGAA |
| 271 | HCV-4966 | 4966 | AUUCAGG | CUGAUGAG | X | CGAA | AAGCCCG | CGGGCUU | A | CCUGAAU |
| 272 | HCV-4974 | 4974 | CCUGGUG | CUGAUGAG | X | CGAA | AUUCAGG | CCUGAAU | A | CACCAGG |
| 273 | HCV-4984 | 4984 | GACGGGC | CUGAUGAG | X | CGAA | ACCCUGG | CCAGGGU | U | GCCCGUC |
| 274 | HCV-4991 | 4991 | CCUGGCA | CUGAUGAG | X | CGAA | ACGGGCA | UGCCCGU | C | UGCCAGG |
| 275 | HCV-5004 | 5004 | AACUCCA | CUGAUGAG | X | CGAA | AUGGUCC | GGACCAU | C | UGGAGUU |
| 276 | HCV-5102 | 5102 | GGUAUGC | CUGAUGAG | X | CGAA | ACCAGGU | ACCUGGU | A | GCAUACC |
| 277 | HCV-5107 | 5107 | GGCUUGG | CUGAUGAG | X | CGAA | AUGCUAC | GUAGCAU | A | CCAAGCC |
| 278 | HCV-5133 | 5133 | GGAGCCU | CUGAUGAG | X | CGAA | AGCCCUG | CAGGGCU | C | AGGCUCC |
| 279 | HCV-5218 | 5218 | UAGCCUA | CUGAUGAG | X | CGAA | ACAGCAG | CUGCUGU | A | UAGGCUA |
| 280 | HCV-5220 | 5220 | CCUAGCC | CUGAUGAG | X | CGAA | AUACAGC | GCUGUAU | A | GGCUAGG |
| 281 | HCV-5306 | 5306 | UAGUGAC | CUGAUGAG | X | CGAA | ACCUCCA | UGGAGGU | C | GUCACUA |
| 282 | HCV-5309 | 5309 | UGCUAGU | CUGAUGAG | X | CGAA | ACGACCU | AGGUCGU | C | ACUACCA |
| 283 | HCV-5313 | 5313 | CAGGUGC | CUGAUGAG | X | CGAA | AGUGACG | CGUCACU | A | GCACCUG |
| 284 | HCV-5330 | 5330 | CUCCGCC | CUGAUGAG | X | CGAA | ACCAGCA | UGCUGGU | A | GGCGGAG |
| 285 | HCV-5339 | 5339 | CUGCAAG | CUGAUGAG | X | CGAA | ACUCCGC | GCGGAGU | C | CUUGCAG |
| 286 | HCV-5342 | 5342 | GAGCUGC | CUGAUGAG | X | CGAA | AGGACUC | GAGUCCU | U | GCAGCUC |
| 287 | HCV-5359 | 5359 | CAGGCAA | CUGAUGAG | X | CGAA | AUGCGGC | GCCGCAU | A | UUGCCUG |
| 288 | HCV-5361 | 5361 | GUCAGGC | CUGAUGAG | X | CGAA | AUAUGCG | CGCAUAU | U | GCCUGAC |
| 289 | HCV-5376 | 5376 | ACCACAC | CUGAUGAG | X | CGAA | ACCGGUU | AACCGGU | A | GUGUGGU |
| 290 | HCV-5399 | 5399 | ACAAAAU | CUGAUGAG | X | CGAA | AUCCUAC | GUAGGAU | C | AUUUUGU |
| 291 | HCV-5423 | 5423 | CGGGAAC | CUGAUGAG | X | CGAA | ACAGCCG | CGGCUGU | U | GUUCCCG |
| 292 | HCV-5426 | 5426 | UGUCGGG | CUGAUGAG | X | CGAA | ACAACAG | CUGUUGU | U | CCCGACA |
| 293 | HCV-5427 | 5427 | CUGUCGG | CUGAUGAG | X | CGAA | AACAACA | UGUUGUU | C | CCGACAG |
| 294 | HCV-5524 | 5524 | CUGCUUG | CUGAUGAG | X | CGAA | ACUGCUC | GAGCAGU | U | CAAGCAG |
| 295 | HCV-5525 | 5525 | UCUGCUU | CUGAUGAG | X | CGAA | AACUGCU | AGCAGUU | C | AAGCAGA |
| 296 | HCV-5583 | 5583 | ACCACGG | CUGAUGAG | X | CGAA | AGCAGCG | CGCUGCU | C | CCGUGGU |
| 297 | HCV-5596 | 5596 | CCACCUG | CUGAUGAG | X | CGAA | ACUCCAC | GUGGAGU | C | CAGGUGG |
| 298 | HCV-5612 | 5612 | AGGCCUC | CUGAUGAG | X | CGAA | AGGGCCC | GGGCCCU | U | GAGGCCU |
| 299 | HCV-5620 | 5620 | UGCCCAG | CUGAUGAG | X | CGAA | AGGCCUC | GAGGCCU | U | CUGGGCA |
| 300 | HCV-5621 | 5621 | UUGCCCA | CUGAUGAG | X | CGAA | AAGGCCU | AGGCCUU | C | UGGGCAA |
| 301 | HCV-5674 | 5674 | AGUGGAU | CUGAUGAG | X | CGAA | AGCCUGC | GCAGGCU | U | AUCCACU |
| 302 | HCV-5675 | 5675 | GAGUGGA | CUGAUGAG | X | CGAA | AAGCCUG | CAGGCUU | A | UCCACUC |
| 303 | HCV-5767 | 5767 | GAUGUUG | CUGAUGAG | X | CGAA | ACAGGAG | CUCCUGU | U | CAACAUC |
| 304 | HCV-5768 | 5768 | AGAUGUU | CUGAUGAG | X | CGAA | AACAGGA | UCCUGUU | C | AACAUCU |
| 305 | HCV-5801 | 5801 | GAGGAGC | CUGAUGAG | X | CGAA | AGUUGAG | CUCAACU | C | GCUCCUC |
| 306 | HCV-5805 | 5805 | CUGGGAG | CUGAUGAG | X | CGAA | AGCGAGU | ACUCGCU | C | CUCCCAG |
| 307 | HCV-5821 | 5821 | GAAGGCC | CUGAUGAG | X | CGAA | AAGCAGC | GCUGCUU | C | GGCCUUC |
| 308 | HCV-5827 | 5827 | GCCCACG | CUGAUGAG | X | CGAA | AGGCCGA | UCGGCCU | U | CGUGGGC |
| 309 | HCV-5828 | 5828 | CGCCCAC | CUGAUGAG | X | CGAA | AAGGCCG | CGGCCUU | C | GUGGGCG |
| 310 | HCV-5843 | 5843 | CACCGGC | CUGAUGAG | X | CGAA | AUGCCGG | CCGGCAU | U | GCCGGUG |
| 311 | HCV-5858 | 5858 | UGCUGCC | CUGAUGAG | X | CGAA | AUGGCCG | CGGCCAU | U | GGCAGCA |
| 312 | HCV-5867 | 5867 | CAAGGCC | CUGAUGAG | X | CGAA | AUCCUCC | GCAGCAU | A | GGCCUUG |
| 313 | HCV-5873 | 5873 | CCUUCCC | CUGAUGAG | X | CGAA | AGGCCUA | UAGGCCU | U | GGGAAGG |
| 314 | HCV-5905 | 5905 | CGCUCCA | CUGAUGAG | X | CGAA | AGCCCGC | GCGGGCU | A | UGGAGCG |
| 315 | HCV-5930 | 5930 | AAGCCAC | CUGAUGAG | X | CGAA | AGUGCAC | GUGCACU | C | GUGGCUU |
| 316 | HCV-5937 | 5937 | ACCUUAA | CUGAUGAG | X | CGAA | AGCCACG | CGUGGCU | U | UUAAGGU |
| 317 | HCV-5938 | 5938 | GACCUUA | CUGAUGAG | X | CGAA | AAGCCAC | GUGGCUU | U | UAAGGUC |
| 318 | HCV-5939 | 5939 | UGACCUU | CUGAUGAG | X | CGAA | AAAGCCA | UGGCUUU | U | AAGGUCA |
| 319 | HCV-5940 | 5940 | AUGACCU | CUGAUGAG | X | CGAA | AAAAGCC | GGCUUUU | A | AGGUCAU |
| 320 | HCV-5945 | 5945 | CGCUCAU | CUGAUGAG | X | CGAA | ACCUUAA | UUAAGGU | C | AUGAGCG |
| 321 | HCV-5965 | 5965 | CUCGGCG | CUGAUGAG | X | CGAA | AGGGCGC | GCGCCCU | C | CGCCGAG |
| 322 | HCV-5981 | 5981 | GCAAGUU | CUGAUGAG | X | CGAA | ACCAGGU | ACCUGGU | U | AACUUGC |
| 323 | HCV-5982 | 5982 | AGCAAGU | CUGAUGAG | X | CGAA | AACCAGG | CCUGGUU | A | ACUUGCU |
| 324 | HCV-5990 | 5990 | UGGCAGG | CUGAUGAG | X | CGAA | AGCAAGU | ACUUGCU | C | CCUGCCA |
| 325 | HCV-6004 | 6004 | GCCGGGG | CUGAUGAG | X | CGAA | AGAGGAU | AUCCUCU | C | CCCCGGC |
| 326 | HCV-6020 | 6020 | CCCCGAC | CUGAUGAG | X | CGAA | ACCAGGG | CCCUGGU | C | GUCGGGG |
| 327 | HCV-6023 | 6023 | CGACCCC | CUGAUGAG | X | CGAA | ACGACCA | UGGUCGU | C | GGGGUCG |
| 328 | HCV-6029 | 6029 | CACACAC | CUGAUGAG | X | CGAA | ACCCCGA | UCGGGGU | C | GUGUGUG |
| 329 | HCV-6044 | 6044 | GACGCAG | CUGAUGAG | X | CGAA | AUUGCUG | CAGCAAU | C | CUGCGUC |
| 330 | HCV-6051 | 6051 | ACGUGCC | CUGAUGAG | X | CGAA | ACGCAGG | CCUGCGU | C | GGCACGU |
| 331 | HCV-6106 | 6106 | CGAAGCG | CUGAUGAG | X | CGAA | ACGCUAU | AUAGCGU | U | CGCUUCG |
| 332 | HCV-6107 | 6107 | GCGAAGC | CUGAUGAG | X | CGAA | AACGCUA | UAGCGUU | C | GCUUCGC |
| 333 | HCV-6111 | 6111 | CCCCGCG | CUGAUGAG | X | CGAA | AGCGAAC | GUUCGCU | U | CGCGGGG |
| 334 | HCV-6413 | 6413 | UUUGCAU | CUGAUGAG | X | CGAA | AUGCCGU | ACGGCAU | C | AUGCAAA |
| 335 | HCV-6574 | 6574 | CCUGGAA | CUGAUGAG | X | CGAA | AGUUCGG | CCGAACU | A | UUCCAGG |
| 336 | HCV-6576 | 6576 | GCCCUGG | CUGAUGAG | X | CGAA | AUAGUUC | GAACUAU | U | CCAGGGC |
| 337 | HCV-6577 | 6577 | CGCCCUG | CUGAUGAG | X | CGAA | AAUAGUU | AACUAUU | C | CAGGGCG |
| 338 | HCV-6637 | 6637 | GUAGUGG | CUGAUGAG | X | CGAA | AGUCCCC | GGGGACU | U | CCACUAC |
| 339 | HCV-6638 | 6638 | CGUAGUG | CUGAUGAG | X | CGAA | AAGUCCC | GGGACUU | C | CACUACG |
| 340 | HCV-6643 | 6643 | CGUCACG | CUGAUGAG | X | CGAA | AGUGGAA | UUCCACU | A | CGUGACG |
| 341 | HCV-6671 | 6671 | GGCAUUU | CUGAUGAG | X | CGAA | ACGUUGU | ACAACGU | A | AAAUGCC |
| 342 | HCV-6703 | 6703 | GGUGAAG | CUGAUGAG | X | CGAA | AUUCGGG | CCCGAAU | U | CUUCACC |
| 343 | HCV-6704 | 6704 | CGGUGAA | CUGAUGAG | X | CGAA | AAUUCGG | CCGAAUU | C | UUCACCG |
| 344 | HCV-6706 | 6706 | UUCGGUG | CUGAUGAG | X | CGAA | AGAAUUC | GAAUUCU | U | CACCGAA |
| 345 | HCV-6707 | 6707 | AUUCGGU | CUGAUGAG | X | CGAA | AAGAAUU | AAUUCUU | C | ACCGAAU |
| 346 | HCV-6715 | 6715 | CCCGUCC | CUGAUGAG | X | CGAA | AUUCGGU | ACCGAAU | U | GGACGGG |
| 347 | HCV-6730 | 6730 | CCUGUGC | CUGAUGAG | X | CGAA | ACCGCAC | GUGCGGU | U | GCACAGG |
| 348 | HCV-6739 | 6739 | CGGAGCG | CUGAUGAG | X | CGAA | ACCUGUG | CACAGGU | A | CGCUCCG |
| 349 | HCV-6744 | 6744 | CACGCCG | CUGAUGAG | X | CGAA | AGCGUAC | GUACGCU | C | CGGCGUG |
| 350 | HCV-6759 | 6759 | CGUAGGA | CUGAUGAG | X | CGAA | AGGUCUG | CAGACCU | C | UCCUACG |
| 351 | HCV-6761 | 6761 | CCCGUAG | CUGAUGAG | X | CGAA | AGAGGUC | GACCUCU | C | CUACGGG |
| 352 | HCV-6764 | 6764 | CCUCCCG | CUGAUGAG | X | CGAA | AGGAGAG | CUCUCCU | A | CGGGAGG |
| 353 | HCV-6776 | 6776 | GGAAUGU | CUGAUGAG | X | CGAA | ACAUCCU | AGGAUGU | C | ACAUUCC |
| 354 | HCV-6782 | 6782 | CGACCUG | CUGAUGAG | X | CGAA | AAUGUGA | UCACAUU | C | CAGGUCG |
| 355 | HCV-6788 | 6788 | UGAGCCC | CUGAUGAG | X | CGAA | ACCUGGA | UCCAGGU | C | GGGCUCA |
| 356 | HCV-6794 | 6794 | AUUGGUU | CUGAUGAG | X | CGAA | AGCCCGA | UCGGGCU | C | AACCAAU |
| 357 | HCV-6802 | 6802 | AACCAGG | CUGAUGAG | X | CGAA | AUUGGUU | AACCAAU | A | CCUGGUU |
| 358 | HCV-6809 | 6809 | GUGACCC | CUGAUGAG | X | CGAA | ACCAGGU | ACCUGGU | U | GGGUCAC |
| 359 | HCV-6814 | 6814 | GAGCUGU | CUGAUGAG | X | CGAA | ACCCAAC | GUUGGGU | C | ACAGCUC |
| 360 | HCV-6821 | 6821 | CGCAUGG | CUGAUGAG | X | CGAA | AGCUGUG | CACAGCU | C | CCAUGCG |
| 361 | HCV-6906 | 6906 | GCCAGCC | CUGAUGAG | X | CGAA | ACGUUUA | UAAACGU | A | GGCUGGC |
| 362 | HCV-6922 | 6922 | GGGGGGA | CUGAUGAG | X | CGAA | ACCCCCU | AGGGGGU | C | UCCCCCC |
| 363 | HCV-6924 | 6924 | GAGGGGG | CUGAUGAG | X | CGAA | AGACCCC | GGGGUCU | C | CCCCCUC |
| 364 | HCV-6931 | 6931 | GGCCAAG | CUGAUGAG | X | CGAA | AGGGGGG | CCCCCCU | C | CUUGGCC |
| 365 | HCV-6934 | 6934 | GCUGGCC | CUGAUGAG | X | CGAA | AGGAGGG | CCCUCCU | U | GGCCAGC |
| 366 | HCV-6943 | 6943 | AGCUGAA | CUGAUGAG | X | CGAA | AGCUGGC | GCCAGCU | C | UUCAGCU |
| 367 | HCV-6958 | 6958 | CGCAGAC | CUGAUGAG | X | CGAA | AUUGGCU | AGCCAAU | U | GUCUGCG |
| 368 | HCV-6961 | 6961 | AGGCGCA | CUGAUGAG | X | CGAA | ACAAUUG | CAAUUGU | C | UGCGCCU |
| 369 | HCV-7034 | 7034 | GCCACAG | CUGAUGAG | X | CGAA | AGGUUGG | CCAACCU | C | CUGUGGC |
| 370 | HCV-7118 | 7118 | CCGCUCG | CUGAUGAG | X | CGAA | AGCGGGU | ACCCGCU | U | CGAGCGG |
| 371 | HCV-7119 | 7119 | UCCGCUC | CUGAUGAG | X | CGAA | AAGCGGG | CCCGCUU | C | GAGCGGA |
| 372 | HCV-7145 | 7145 | CAACGGA | CUGAUGAG | X | CGAA | ACUUCCC | GGGAAGU | A | UCCGUUG |
| 373 | HCV-7195 | 7195 | UAUGGGC | CUGAUGAG | X | CGAA | ACGCGGG | CCCGCGU | U | GCCCAUA |
| 374 | HCV-7202 | 7202 | GUGCCCA | CUGAUGAG | X | CGAA | AUGGGCA | UGCCCAU | A | UGGGCAC |
| 375 | HCV-7218 | 7213 | GGGUUGU | CUGAUGAG | X | CGAA | AUCCGGG | CCCGGAU | U | ACAACCC |
| 376 | HCV-7219 | 7219 | AGGGUUG | CUGAUGAG | X | CGAA | AAUCCGG | CCGGAUU | A | CAACCCU |
| 377 | HCV-7234 | 7234 | GGACUCU | CUGAUGAG | X | CGAA | ACAGUGG | CCACUGU | U | AGAGUCC |
| 378 | HCV-7235 | 7235 | AGGACUC | CUGAUGAG | X | CGAA | AACAGUG | CACUGUU | A | GAGUCCU |
| 379 | HCV-7251 | 7251 | UAGUCCG | CUGAUGAG | X | CGAA | ACUUUUC | GAAAAGU | C | CGGACUA |
| 380 | HCV-7258 | 7258 | AGGGACG | CUGAUGAG | X | CGAA | AGUCCGG | CCGGACU | A | CGUCCCU |
| 381 | HCV-7262 | 7262 | CCGGAGG | CUGAUGAG | X | CGAA | ACGUAGU | ACUACGU | C | CCUCCGG |
| 382 | HCV-7266 | 7266 | ACCGCCG | CUGAUGAG | X | CGAA | AGGGACG | CGUCCCU | C | CGGCGGU |
| 383 | HCV-7288 | 7288 | AGGCGGC | CUGAUGAG | X | CGAA | AUGGGCA | UGCCCAU | U | GCCGCCU |
| 384 | HCV-7296 | 7296 | CCCGUGG | CUGAUGAG | X | CGAA | AGGCGGC | GCCGCCU | A | CCACGGG |
| 385 | HCV-7354 | 7354 | CACGGUG | CUGAUGAG | X | CGAA | ACUCUGU | ACAGAGU | C | CACCGUG |
| 386 | HCV-7386 | 7386 | GUCUUAG | CUGAUGAG | X | CGAA | AGCCAGC | GCUGGCU | A | CUAAGAC |
| 387 | HCV-7389 | 7389 | AAAGUCU | CUGAUGAG | X | CGAA | AGUAGCC | G3CUACU | A | AGACUUU |
| 388 | HCV-7395 | 7395 | CUGCCGA | CUGAUGAG | X | CGAA | AGUCUUA | UAAGACU | U | UCGGCAG |
| 389 | HCV-7396 | 7396 | GCUGCCG | CUGAUGAG | X | CGAA | AAGUCUU | AAGACUU | U | CGGCAGC |
| 390 | HCV-7397 | 7397 | AGCUGCC | CUGAUGAG | X | CGAA | AAAGUCU | AGACUUU | C | GGCAGCU |
| 391 | HCV-7411 | 7411 | GCCCGAC | CUGAUGAG | X | CGAA | AUCCGGA | UCCGGAU | C | GUCGGCC |
| 392 | HCV-7414 | 7414 | AACGGCC | CUGAUGAG | X | CGAA | ACGAUCC | GGAUCGU | C | GGCCGUU |
| 393 | HCV-7421 | 7421 | CGCUGUC | CUGAUGAG | X | CGAA | ACGGCCG | CGGCCGU | U | GACAGCG |
| 394 | HCV-7498 | 7498 | CAUGGAG | CUGAUGAG | X | CGAA | AGUACGA | UCGUACU | C | CUCCAUG |
| 395 | HCV-7501 | 7501 | GGGCAUG | CUGAUGAG | X | CGAA | AGGAGUA | UACUCCU | C | CAUGCCC |
| 396 | HCV-7514 | 7514 | CCCCCUC | CUGAUGAG | X | CGAA | AGGGGGG | CCCCCCU | U | GAGGGGG |
| 397 | HCV-7539 | 7539 | UCGCUGA | CUGAUGAG | X | CGAA | AUCAGGG | CCCUGAU | C | UCAGCGA |
| 398 | HCV-7541 | 7541 | CGUCGCU | CUGAUGAG | X | CGAA | AGAUCAG | CUGAUCU | C | AGCGACG |
| 399 | HCV-7552 | 7552 | AGACCAA | CUGAUGAG | X | CGAA | ACCCGUC | GACGGGU | C | UUGGUCU |
| 400 | HCV-7554 | 7554 | GUAGACC | CUGAUGAG | X | CGAA | AGACCCG | CGGGUCU | U | GGUCUAC |
| 401 | HCV-7558 | 7558 | CACGGUA | CUGAUGAG | X | CGAA | ACCAAGA | UCUUGGU | C | UACCGUG |
| 402 | HCV-7560 | 7560 | CUCACGG | CUGAUGAG | X | CGAA | AGACCAA | UUGGUCU | A | CCGUGAG |
| 403 | HCV-7589 | 7589 | AGCAGAC | CUGAUGAG | X | CGAA | AUGUCGU | ACGACAU | C | GUCUGCU |
| 404 | HCV-7592 | 7592 | AGCAGCA | CUGAUGAG | X | CGAA | ACGAUGU | ACAUCGU | C | UGCUGCU |
| 405 | HCV-7600 | 7600 | GGACAUU | CUGAUGAG | X | CGAA | AGCAGCA | UGCUGCU | C | AAUGUCC |
| 406 | HCV-7606 | 7606 | UGUGUAG | CUGAUGAG | X | CGAA | ACAUUGA | UCAAUGU | C | CUACACA |
| 407 | HCV-7667 | 7667 | ACGCGUU | CUGAUGAG | X | CGAA | AUGGGCA | UGCCCAU | C | AACGCGU |
| 408 | HCV-7723 | 7723 | ACUGCGG | CUGAUGAG | X | CGAA | AUGUUGU | ACAACAU | C | CCGCAGU |
| 409 | HCV-7775 | 7775 | CGUCCAG | CUGAUGAG | X | CGAA | ACUUGCA | UGCAAGU | C | CUGGACG |
| 410 | HCV-7789 | 7789 | GUCCCGG | CUGAUGAG | X | CGAA | AGUGGUC | GACCACU | A | CCGGGAC |
| 411 | HCV-7839 | 7839 | AGAAGUU | CUGAUGAG | X | CGAA | AGCCUUA | UAAGGCU | A | AACUUCU |
| 412 | HCV-7847 | 7847 | CUACGGA | CUGAUGAG | X | CGAA | AGAAGUU | AACUUCU | A | UCCGUAG |
| 413 | HCV-7849 | 7849 | UUCUACG | CUGAUGAG | X | CGAA | AUAGAAG | CUUCUAU | C | CGUAGAA |
| 414 | HCV-7853 | 7853 | CUUCUUC | CUGAUGAG | X | CGAA | ACGGAUA | UAUCCGU | A | GAAGAAG |
| 415 | HCV-7894 | 7894 | AAAUUUA | CUGAUGAG | X | CGAA | AUUUGGC | GCCAAAU | C | UAAAUUU |
| 416 | HCV-7896 | 7896 | CCAAAUU | CUGAUGAG | X | CGAA | AGAUUUG | CAAAUCU | A | AAUUUGG |
| 417 | HCV-7900 | 7900 | AUAGCCA | CUGAUGAG | X | CGAA | AUUUAGA | UCUAAAU | U | UGGCUAU |
| 418 | HCV-7901 | 7901 | CAUAGCC | CUGAUGAG | X | CGAA | AAUUUAG | CUAAAUU | U | GGCUAUG |
| 419 | HCV-7906 | 7906 | UGCCCCA | CUGAUGAG | X | CGAA | AGCCAAA | UUUGGCU | A | UGGGGCA |
| 420 | HCV-7955 | 7955 | CGGAGCG | CUGAUGAG | X | CGAA | AUGUGGU | ACCACAU | C | CGCUCCG |
| 421 | HCV-7960 | 7960 | CCACACG | CUGAUGAG | X | CGAA | AGCGGAU | AUCCGCU | C | CGUGUGG |
| 422 | HCV-8075 | 8075 | AUACGAU | CUGAUGAG | X | CGAA | AGGCGAG | CUCGCCU | U | AUCGUAU |
| 423 | HCV-8076 | 8076 | AAUACGA | CUGAUGAG | X | CGAA | AAGGCGA | UCGCCUU | A | UCGUAUU |
| 424 | HCV-8078 | 8078 | GGAAUAC | CUGAUGAG | X | CGAA | AUAAGGC | GCCUUAU | C | GUAUUCC |
| 425 | HCV-8170 | 8170 | GAAUCCG | CUGAUGAG | X | CGAA | ACGAGGA | UCCUCGU | A | CGGAUUC |
| 426 | HCV-8176 | 8176 | GUACUGG | CUGAUGAG | X | CGAA | AUCCGUA | UACGGAU | U | CCAGUAC |
| 427 | HCV-8182 | 8182 | AGGAGAG | CUGAUGAG | X | CGAA | ACUGGAA | UUCCAGU | A | CUCUCCU |
| 428 | HCV-8187 | 8187 | UGCCCAG | CUGAUGAG | X | CGAA | AGAGUAC | GUACUCU | C | CUGGGCA |
| 429 | HCV-8201 | 8201 | GGAACUC | CUGAUGAG | X | CGAA | ACCCGCU | AGCGGGU | U | GAGUUCC |
| 430 | HCV-8206 | 8206 | CACCAGG | CUGAUGAG | X | CGAA | ACUCAAC | GUUGAGU | U | CCUGGUG |
| 431 | HCV-8207 | 8207 | UCACCAG | CUGAUGAG | X | CGAA | AACUCAA | UUGAGUU | C | CUGGUGA |
| 432 | HCV-8227 | 8227 | UUUCUUU | CUGAUGAG | X | CGAA | AUUUCCA | UGGAAAU | C | AAAGAAA |
| 433 | HCV-8357 | 8357 | GCGACUU | CUGAUGAG | X | CGAA | AUGGCCU | AGGCCAU | A | AAGUCGC |
| 434 | HCV-8362 | 8362 | CGUGAGC | CUGAUGAG | X | CGAA | ACUUUAU | AUAAAGU | C | GCUCACG |
| 435 | HCV-8366 | 8366 | GCUCCGU | CUGAUGAG | X | CGAA | AGCGACU | AGUCGCU | C | ACGGAGC |
| 436 | HCV-8378 | 8378 | CGAUGUA | CUGAUGAG | X | CGAA | AGCCGCU | AGCGGCU | C | UACAUCG |
| 437 | HCV-8380 | 8380 | CCCGAUG | CUGAUGAG | X | CGAA | AGAGCCG | CGGCUCU | A | CAUCGGG |
| 438 | HCV-8384 | 8384 | GGCCCCC | CUGAUGAG | X | CGAA | AUGUAGA | UCUACAU | C | GGGGGCC |
| 439 | HCV-8424 | 8424 | CGGCGAU | CUGAUGAG | X | CGAA | ACCGCAG | CUGCGGU | U | AUCGCCG |
| 440 | HCV-8425 | 8425 | CCGGCGA | CUGAUGAG | X | CGAA | AACCGCA | UGCGGUU | A | UCGCCGG |
| 441 | HCV-8427 | 8427 | CACCGGC | CUGAUGAG | X | CGAA | AUAACCG | CGGUUAU | C | GCCGGUG |
| 442 | HCV-8460 | 8460 | CCGCAGC | CUGAUGAG | X | CGAA | AGUCGUC | GACGACU | A | GCUGCGG |
| 443 | HCV-8508 | 8508 | GCAGCUC | CUGAUGAG | X | CGAA | ACAGGCC | GGCCUGU | C | GAGCUGC |
| 444 | HCV-8522 | 8522 | AGUCCUG | CUGAUGAG | X | CGAA | AGCUUUG | CAAAGCU | C | CAGGACU |
| 445 | HCV-8540 | 8540 | CGUUCAC | CUGAUGAG | X | CGAA | AGCAUCG | CGAUGCU | C | GUGAACG |
| 446 | HCV-8558 | 8558 | UAACGAC | CUGAUGAG | X | CGAA | AGGUCGU | ACGACCU | U | GUCGUUA |
| 447 | HCV-8561 | 8561 | AGAUAAC | CUGAUGAG | X | CGAA | ACAAGGU | ACCUUGU | C | GUUAUCU |
| 448 | HCV-8564 | 8564 | CACAGAU | CUGAUGAG | X | CGAA | ACGACAA | UUGUCGU | U | AUCUGUG |
| 449 | HCV-8638 | 8638 | GGGGGCA | CUGAUGAG | X | CGAA | AGUACCU | AGGUACU | C | UGCCCCC |
| 450 | HCV-8671 | 8671 | CAAGUCG | CUGAUGAG | X | CGAA | AUUCUGG | CCAGAAU | A | CGACUUG |
| 451 | HCV-8698 | 8698 | GUUGGAG | CUGAUGAG | X | CGAA | AGCAUGA | UCAUGCU | C | CUCCAAC |
| 452 | HCV-8701 | 8701 | CACGUUG | CUGAUGAG | X | CGAA | AGGAGCA | UGCUCCU | C | CAACGUG |
| 453 | HCV-8728 | 8728 | UUUGCCG | CUGAUGAG | X | CGAA | AUGCGUC | GACGCAU | C | CGGCAAA |
| 454 | HCV-8774 | 8774 | CCCGUGC | CUGAUGAG | X | CGAA | AGGGGGG | CCCCCCU | U | GCACGGG |
| 455 | HCV-8842 | 8842 | GGGCGCA | CUGAUGAG | X | CGAA | ACAUGAU | AUCAUGU | A | UGCGCCC |
| 456 | HCV-8854 | 8854 | UGCCCAU | CUGAUGAG | X | CGAA | AGGUGGG | CCCACCU | U | AUGGGCA |
| 457 | HCV-8855 | 8855 | UUGCCCA | CUGAUGAG | X | CGAA | AAGGUGG | CCACCUU | A | UGGGCAA |
| 458 | HCV-8871 | 8871 | GUCAUCA | CUGAUGAG | X | CGAA | AAUCAUC | GAUGAUU | U | UGAUGAC |
| 459 | HCV-8880 | 8880 | AAGAAGU | CUGAUGAG | X | CGAA | AGUCAUC | GAUGACU | C | ACUUCUU |
| 460 | HCV-8931 | 8931 | AUCUGAC | CUGAUGAG | X | CGAA | AUCCAGG | CCUGGAU | U | GUCAGAU |
| 461 | HCV-8934 | 8934 | UAGAUCU | CUGAUGAG | X | CGAA | ACAAUCC | GGAUUGU | C | AGAUCUA |
| 462 | HCV-8939 | 8939 | CCCCGUA | CUGAUGAG | X | CGAA | AUCUGAC | GUCAGAU | C | UACGGGG |
| 463 | HCV-8941 | 8941 | GGCCCCG | CUGAUGAG | X | CGAA | AGAUCUG | CAGAUCU | A | CGGGGCC |
| 464 | HCV-9065 | 9065 | GUUUCCU | CUGAUGAG | X | CGAA | AGGCAUG | CAUGCCU | C | AGGAAAC |
| 465 | HCV-9074 | 9074 | GUACCCC | CUGAUGAG | X | CGAA | AGUUUCC | GGAAACU | U | GGGGUAC |
| 466 | HCV-9080 | 9080 | AGGGCGG | CUGAUGAG | X | CGAA | ACCCCAA | UUGGGGU | A | CCGCCCU |
| 467 | HCV-9088 | 9088 | GACUCGC | CUGAUGAG | X | CGAA | AGGGCGG | CCGCCCU | U | GCGAGUC |
| 468 | HCV-9095 | 9095 | GUCUCCA | CUGAUGAG | X | CGAA | ACUCGCA | UGCGAGU | C | UGGAGAC |
| 469 | HCV-9119 | 9119 | UAGCGCG | CUGAUGAG | X | CGAA | ACACUUC | GAAGUGU | C | CGCGCUA |
| 470 | HCV-9126 | 9126 | AGUAGCC | CUGAUGAG | X | CGAA | AGCGCGG | CCGCGCU | A | GGCUACU |
| 471 | HCV-9131 | 9131 | GGGACAG | CUGAUGAG | X | CGAA | AGCCUAG | CUAGGCU | A | CUGUCCC |
| 472 | HCV-9136 | 9136 | CCCUUGG | CUGAUGAG | X | CGAA | ACAGUAG | CUACUGU | C | CCAAGGG |
| 473 | HCV-9226 | 9226 | CAGCUGG | CUGAUGAG | X | CGAA | ACGCGGC | GCCGCGU | C | CCAGCUG |
| 474 | HCV-9238 | 9238 | GCUGGAC | CUGAUGAG | X | CGAA | AGUCCAG | CUGGACU | U | GUCCAGC |
| 475 | HCV-9241 | 9241 | CCAGCUG | CUGAUGAG | X | CGAA | ACAAGUC | GACUUGU | C | CAGCUGG |
| 476 | HCV-9250 | 9250 | AGCAACG | CUGAUGAG | X | CGAA | ACCAGCU | AGCUGGU | U | CGUUGCU |
| 477 | HCV-9251 | 9251 | CAGCAAC | CUGAUGAG | X | CGAA | AACCAGC | GCUGGUU | C | GUUGCUG |
| 478 | HCV-9254 | 9254 | AACCAGC | CUGAUGAG | X | CGAA | ACGAACC | GGUUCGU | U | GCUGGUU |
| 479 | HCV-9278 | 9278 | UGUGAUA | CUGAUGAG | X | CGAA | AUGUCUC | GAGACAU | A | UAUCACA |
| 480 | HCV-9280 | 9280 | GCUGUGA | CUGAUGAG | X | CGAA | AUAUGUC | GACAUAU | A | UCACAGC |
| 481 | HCV-9282 | 9282 | AGGCUGU | CUGAUGAG | X | CGAA | AUAUAUG | CAUAUAU | C | ACAGCCU |
| 482 | HCV-9292 | 9292 | GGCACGA | CUGAUGAG | X | CGAA | ACAGGCU | AGCCUGU | C | UCGUGCC |
| 483 | HCV-9326 | 9326 | GUAGGAG | CUGAUGAG | X | CGAA | AGGCACC | GGUGCCU | A | CUCCUAC |
| 484 | HCV-9329 | 9329 | AAAGUAG | CUGAUGAG | X | CGAA | AGUAGGC | GCCUACU | C | CUACUUU |
| 485 | HCV-9332 | 9332 | CGGAAAG | CUGAUGAG | X | CGAA | AGGAGUA | UACUCCU | A | CUUUCCG |
| 486 | HCV-9335 | 9335 | CUACGGA | CUGAUGAG | X | CGAA | AGUAGGA | UCCUACU | U | UCCGUAG |
| 487 | HCV-9336 | 9336 | CCUACGG | CUGAUGAG | X | CGAA | AAGUAGG | CCUACUU | U | CCGUAGG |
| 488 | HCV-9337 | 9337 | CCCUACG | CUGAUGAG | X | CGAA | AAAGUAG | CUACUUU | C | CGUAGGG |
| 489 | HCV-9341 | 9341 | CUACCCC | CUGAUGAG | X | CGAA | ACGGAAA | UUUCCGU | A | GGGGUAG |
| 490 | HCV-9347 | 9347 | AGAUGCC | CUGAUGAG | X | CGAA | ACCCCUA | UAGGGGU | A | GGCAUCU |
| 491 | HCV-9353 | 9353 | GCAGGUA | CUGAUGAG | X | CGAA | AUGCCUA | UAGGCAU | C | UACCUGC |
| 492 | HCV-9355 | 9355 | GAGCAGG | CUGAUGAG | X | CGAA | AGAUGCC | GGCAUCU | A | CCUGCUC |
| 493 | HCV-9362 | 9362 | GGUUGGG | CUGAUGAG | X | CGAA | AGCAGGU | ACCUGCU | C | CCCAACC |
| 494 | HCV-9385 | 9385 | GAGUGAU | CUGAUGAG | X | CGAA | AGCUCCC | GGGAGCU | A | AUCACUC |
| 495 | HCV-9388 | 9388 | CUGGAGU | CUGAUGAG | X | CGAA | AUUAGCU | AGCUAAU | C | ACUCCAG |
| 496 | HCV-9392 | 9392 | UGGCCUG | CUGAUGAG | X | CGAA | AGUGAUU | AAUCACU | C | CAGGCCA |
| 497 | HCV-9402 | 9402 | GAUGGCC | CUGAUGAG | X | CGAA | AUUGGCC | GGCCAAU | A | GGCCAUC |

Where “X” represents stem II region of a HH ribozyme (Hertel et al., 1992 Nucleic Acids Res. 20:3252). The length of stem II may be 2 base-pairs.

TABLE VI
Additional HCV Hammerhead (HH) Ribozyme and Target Sequence

| Pos. | Ribozyme | | | | | Substrate | | |
|---|---|---|---|---|---|---|---|---|
| 14 | CGCCCCC | CUGAUGAG | X | CGAA | AUCGGGG | CCCCGAU | U | GGGGGCG |
| 34 | AGUGAUC | CUGAUGAG | X | CGAA | AUGGUGG | CCACCAU | A | GAUCACU |
| 38 | GGGGAGU | CUGAUGAG | X | CGAA | AUCUAUG | CAUAGAU | C | ACUCCCC |
| 42 | CACAGGG | CUGAUGAG | X | CGAA | AGUGAUC | GAUCACU | C | CCCUGUG |
| 57 | AAGACAG | CUGAUGAG | X | CGAA | AGUUCCU | AGGAACU | A | CUGUCUU |
| 62 | GCGUGAA | CUGAUGAG | X | CGAA | ACAGUAG | CUACUGU | C | UUCACGC |
| 64 | CUGCGUG | CUGAUGAG | X | CGAA | AGACAGU | ACUGUCU | U | CACGCAG |
| 65 | UCUGCGU | CUGAUGAG | X | CGAA | AAGACAG | CUGUCUU | C | ACGCAGA |
| 79 | AUGGCUA | CUGAUGAG | X | CGAA | ACGCUUU | AAAGCGU | C | UAGCCAU |
| 81 | CCAUGGC | CUGAUGAG | X | CGAA | AGACGCU | AGCGUCU | A | GCCAUGG |
| 92 | UCAUACU | CUGAUGAG | X | CGAA | ACGCCAU | AUGGCGU | U | AGUAUGA |
| 93 | CUCAUAC | CUGAUGAG | X | CGAA | AACGCCA | UGGCGUU | A | GUAUGAG |
| 96 | ACACUCA | CUGAUGAG | X | CGAA | ACUAACG | CGUUAGU | A | UGAGUGU |
| 104 | GCUGCAC | CUGAUGAG | X | CGAA | ACACUCA | UGAGUGU | C | GUGCAGC |
| 142 | AGACCAC | CUGAUGAG | X | CGAA | AUGGCUC | GAGCCAU | A | GUGGUCU |
| 192 | AAGAAAG | CUGAUGAG | X | CGAA | ACCCGGU | ACCGGGU | C | CUUUCUU |
| 195 | UCCAAGA | CUGAUGAG | X | CGAA | AGGACCC | GGGUCCU | U | UCUUGGA |
| 196 | AUCCAAG | CUGAUGAG | X | CGAA | AAGGACC | GGUCCUU | U | CUUGGAU |
| 197 | GAUCCAA | CUGAUGAG | X | CGAA | AAAGGAC | GUCCUUU | C | UUGGAUC |
| 204 | GCGGGUU | CUGAUGAG | X | CGAA | AUCCAAG | CUUGGAU | C | AACCCGC |
| 227 | ACGCCCA | CUGAUGAG | X | CGAA | AUCUCCA | UGGAGAU | U | UGGGCGU |
| 228 | CACGCCC | CUGAUGAG | X | CGAA | AAUCUCC | GGAGAUU | U | GGGCGUG |
| 282 | GUACCAC | CUGAUGAG | X | CGAA | AGGCCUU | AAGGCCU | U | GUGGUAC |
| 354 | GGUUUAG | CUGAUGAG | X | CGAA | AUUCGUG | CACGAAU | C | CUAAACC |
| 357 | UGAGGUU | CUGAUGAG | X | CGAA | AGGAUUC | GAAUCCU | A | AACCUCA |
| 363 | UUUCUUU | CUGAUGAG | X | CGAA | AGGUUUA | UAAACCU | C | AAAGAAA |
| 381 | UAGGUGU | CUGAUGAG | X | CGAA | ACGUUUG | CAAACGU | A | ACACCUA |
| 388 | GCGGCGG | CUGAUGAG | X | CGAA | AGGUGUU | AACACCU | A | CCGCCGC |
| 431 | CACCAAC | CUGAUGAG | X | CGAA | AUCUGAC | GUCAGAU | C | GUUGGUG |
| 434 | CUCCACC | CUGAUGAG | X | CGAA | ACGAUCU | AGAUCGU | U | GGUGGAG |
| 443 | ACACGUA | CUGAUGAG | X | CGAA | ACUCCAC | GUGGAGU | U | UACGUGU |
| 444 | AACACGU | CUGAUGAG | X | CGAA | AACUCCA | UGGAGUU | U | ACGUGUU |
| 445 | CAACACG | CUGAUGAG | X | CGAA | AAACUCC | GGAGUUU | A | CGUGUUG |
| 451 | GCGCGGC | CUGAUGAG | X | CGAA | ACACGUA | UACGUGU | U | GCCGCGC |
| 516 | CUUCCAC | CUGAUGAG | X | CGAA | AGGUUGC | GCAACCU | C | GUGGAAG |
| 688 | AUUGCGC | CUGAUGAG | X | CGAA | ACCUCCG | CGGAGGU | C | GCGCAAU |
| 702 | AUGACCU | CUGAUGAG | X | CGAA | ACCCAGA | UCUGGGU | A | AGGUCAU |
| 719 | CGCACGU | CUGAUGAG | X | CGAA | AGGGUAU | AUACCCU | C | ACGUGCG |
| 740 | ACCCCAU | CUGAUGAG | X | CGAA | AGGUCGG | CCGACCU | C | AUGGGGU |
| 861 | AUAGAGA | CUGAUGAG | X | CGAA | AGAGCAA | UUGCUCU | U | UCUCUAU |
| 862 | GAUAGAG | CUGAUGAG | X | CGAA | AAGAGCA | UGCUCUU | U | CUCUAUC |
| 863 | AGAUAGA | CUGAUGAG | X | CGAA | AAAGAGC | GCUCUUU | C | UCUAUCU |
| 865 | GAAGAUA | CUGAUGAG | X | CGAA | AGAAAGA | UCUUUCU | C | UAUCUUC |
| 867 | AGGAAGA | CUGAUGAG | X | CGAA | AGAGAAA | UUUCUCU | A | UCUUCCU |
| 869 | AGAGGAA | CUGAUGAG | X | CGAA | AUAGAGA | UCUCUAU | C | UUCCUCU |
| 871 | CAAGAGG | CUGAUGAG | X | CGAA | AGAUAGA | UCUAUCU | U | CCUCUUG |
| 872 | CCAAGAG | CUGAUGAG | X | CGAA | AAGAUAG | CUAUCUU | C | CUCUUGG |
| 875 | GGGCCAA | CUGAUGAG | X | CGAA | AGGAAGA | UCUUCCU | C | UUGGCCC |
| 877 | CAGGGCC | CUGAUGAG | X | CGAA | AGAGGAA | UUCCUCU | U | GGCCCUG |
| 889 | CAAACAG | CUGAUGAG | X | CGAA | ACAGCAG | CUGCUGU | C | CUGUUUG |
| 894 | AUGGUCA | CUGAUGAG | X | CGAA | ACAGGAC | GUCCUGU | U | UGACCAU |
| 895 | GAUGGUC | CUGAUGAG | X | CGAA | AACAGGA | UCCUGUU | U | GACCAUC |
| 902 | AAGCUGG | CUGAUGAG | X | CGAA | AUGGUCA | UGACCAU | C | CCAGCUU |
| 909 | UAAGCGG | CUGAUGAG | X | CGAA | AGCUGGG | CCCAGCU | U | CCGCUUA |
| 910 | AUAAGCG | CUGAUGAG | X | CGAA | AAGCUGG | CCAGCUU | C | CGCUUAU |
| 915 | ACCUGAU | CUGAUGAG | X | CGAA | AGCGGAA | UUCCGCU | U | AUCAGGU |
| 916 | CACCUGA | CUGAUGAG | X | CGAA | AAGCGGA | UCCGCUU | A | UCAGGUG |
| 918 | CGCACCU | CUGAUGAG | X | CGAA | AUAAGCG | CGCUUAU | C | AGGUGCG |
| 934 | CAGCCCG | CUGAUGAG | X | CGAA | AUGCGUU | AACGCAU | C | CGGGCUG |
| 943 | GACAUGG | CUGAUGAG | X | CGAA | ACAGCCC | GGGCUGU | A | CCAUGUC |
| 950 | CAUUCGU | CUGAUGAG | X | CGAA | ACAUGGU | ACCAUGU | C | ACGAAUG |
| 964 | UGAGUUG | CUGAUGAG | X | CGAA | AGCAGUC | GACUGCU | C | CAACUCA |
| 970 | AAUGCUU | CUGAUGAG | X | CGAA | AGUUGGA | UCCAACU | C | AAGCAUU |
| 977 | CAUACAC | CUGAUGAG | X | CGAA | AUGCUUG | CAAGCAU | U | GUGUAUG |
| 1008 | CCGGGGG | CUGAUGAG | X | CGAA | AUGCAUG | CAUGCAU | A | CCCCCGG |
| 1067 | UGGGAGU | CUGAUGAG | X | CGAA | AGCGCUA | UAGCGCU | C | ACUCCCA |
| 1071 | AGCGUGG | CUGAUGAG | X | CGAA | AGUGAGC | GCUCACU | C | CCACGCU |
| 1079 | UGGCCGC | CUGAUGAG | X | CGAA | AGCGUGG | CCACGCU | C | GCGGCCA |
| 1100 | UAGUGGG | CUGAUGAG | X | CGAA | AUGCUGG | CCAGCAU | C | CCCACUA |
| 1107 | AUUGUCG | CUGAUGAG | X | CGAA | AGUGGGG | CCCCACU | A | CGACAAU |
| 1115 | GGCGUCG | CUGAUGAG | X | CGAA | AUUGUCG | CGACAAU | A | CGACGCC |
| 1152 | GAACAGA | CUGAUGAG | X | CGAA | AGCGGCC | GGCCGCU | U | UCUGUUC |
| 1181 | AUCCGCA | CUGAUGAG | X | CGAA | AGGUCCC | GGGACCU | C | UGCGGAU |
| 1199 | GGGAGAC | CUGAUGAG | X | CGAA | AGGAAAA | UUUUCCU | C | GUCUCCC |
| 1202 | ACUGGGA | CUGAUGAG | X | CGAA | ACGAGGA | UCCUCGU | C | UCCCAGU |
| 1204 | CAACUGG | CUGAUGAG | X | CGAA | AGACGAG | CUCGUCU | C | CCAGUUG |
| 1210 | GGUGAAC | CUGAUGAG | X | CGAA | ACUGGGA | UCCCAGU | U | GUUCACC |
| 1213 | GAAGGUG | CUGAUGAG | X | CGAA | ACAACUG | CAGUUGU | U | CACCUUC |
| 1214 | AGAAGGU | CUGAUGAG | X | CGAA | AACAACU | AGUUGUU | C | ACCUUCU |
| 1219 | AGGCGAG | CUGAUGAG | X | CGAA | AGGUGAA | UUCACCU | U | CUCGCCU |
| 1220 | GAGGCGA | CUGAUGAG | X | CGAA | AAGGUGA | UCACCUU | C | UCGCCUC |
| 1222 | GCGAGGC | CUGAUGAG | X | CGAA | AGAAGGU | ACCUUCU | C | GCCUCGC |
| 1227 | UACCGGC | CUGAUGAG | X | CGAA | AGGCGAG | CUCGCCU | C | GCCGGUA |
| 1234 | UGUCUCA | CUGAUGAG | X | CGAA | ACCGGCG | CGCCGGU | A | UGAGACA |
| 1244 | AGUCCUG | CUGAUGAG | X | CGAA | ACUGUCU | AGACAGU | A | CAGGACU |
| 1257 | AUUGAGC | CUGAUGAG | X | CGAA | AUUGCAG | CUGCAAU | U | GCUCAAU |
| 1261 | AUAGAUU | CUGAUGAG | X | CGAA | AGCAAUU | AAUUGCU | C | AAUCUAU |
| 1265 | CGGGAUA | CUGAUGAG | X | CGAA | AUUGAGC | GCUCAAU | C | UAUCCCG |
| 1267 | GCCGGGA | CUGAUGAG | X | CGAA | AGAUGGA | UCAAUCU | A | UCCCGGC |
| 1269 | UGGCCGG | CUGAUGAG | X | CGAA | AUAGAUU | AAUCUAU | C | CCGGCCA |
| 1299 | AUAUCCC | CUGAUGAG | X | CGAA | AGCCAUG | CAUGGCU | U | GGGAUAU |
| 1305 | AUCAUCA | CUGAUGAG | X | CGAA | AUCCCAA | UUGGGAU | A | UGAUGAU |
| 1321 | UGUAGGC | CUGAUGAG | X | CGAA | ACCAGUU | AACUGGU | C | GCCUACA |
| 1326 | GCUGUUG | CUGAUGAG | X | CGAA | AGGCGAC | GUCGCCU | A | CAACAGC |
| 1337 | ACACCAC | CUGAUGAG | X | CGAA | AGGGCUG | CAGCCCU | A | GUGGUGU |
| 1345 | UAACUGC | CUGAUGAG | X | CGAA | ACACCAC | GUGGUGU | C | GCAGUUA |
| 1351 | CCGGAGU | CUGAUGAG | X | CGAA | ACUGCGA | UCGCAGU | U | ACUCCGG |
| 1352 | UCCGGAG | CUGAUGAG | X | CGAA | AACUGCG | CGCAGUU | A | CUCCGGA |
| 1355 | GGAUCCG | CUGAUGAG | X | CGAA | AGUAACU | AGUUACU | C | CGGAUCC |
| 1361 | CUGGUGG | CUGAUGAG | X | CGAA | AUCCGGA | UCCGGAU | C | CCACAAG |
| 1449 | AAGACCU | CUGAUGAG | X | CGAA | AGCCCAG | CUGGGCU | A | AGGUCUU |
| 1454 | CAAUCAA | CUGAUGAG | X | CGAA | ACCUUAG | CUAAGGU | C | UUGAUUG |
| 1456 | CACAAUC | CUGAUGAG | X | CGAA | AGACCUC | AAGGUCU | U | GAUUGUG |
| 1460 | ACAUCAC | CUGAUGAG | X | CGAA | AUCAAGA | UCUUGAU | U | GUGAUGU |
| 1468 | AAAGAGU | CUGAUGAG | X | CGAA | ACAUCAC | GUGAUGU | U | ACUCUUU |
| 1469 | CAAAGAG | CUGAUGAG | X | CGAA | AACAUCA | UGAUGUU | A | CUCUUUG |
| 1472 | CGGCAAA | CUGAUGAG | X | CGAA | AGUAACA | UGUUACU | C | UUUGCCG |
| 1474 | GCCGGCA | CUGAUGAG | X | CGAA | AGAGUAA | UUACUCU | U | UGCCGGC |
| 1475 | CGCCGGC | CUGAUGAG | X | CGAA | AAGAGUA | UACUCUU | U | GCCGGCG |
| 1484 | CCCCGUC | CUGAUGAG | X | CGAA | ACGCCGG | CCGGCGU | U | GACGGGG |
| 1493 | UGUAAGU | CUGAUGAG | X | CGAA | ACCCCGU | ACGGGGU | C | ACUUACA |
| 1497 | GUCGUGU | CUGAUGAG | X | CGAA | AGUGACC | GGUCACU | U | ACACGAC |
| 1498 | UGUCGUG | CUGAUGAG | X | CGAA | AAGUGAC | GUCACUG | A | CACGACA |
| 1513 | AGCUUGC | CUGAUGAG | X | CGAA | ACCCCCC | GGGGGGU | C | GCAAGCU |
| 1521 | GUGUGGC | CUGAUGAG | X | CGAA | AGCUUGC | GCAAGCU | C | GCCACAC |
| 1538 | AGGACGU | CUGAUGAG | X | CGAA | ACGCUCU | AGAGCGU | C | ACGUCCU |
| 1543 | GAAGAAG | CUGAUGAG | X | CGAA | ACGUGAC | GUCACGU | C | CUUCUUC |
| 1546 | GGUGAAG | CUGAUGAG | X | CGAA | AGGACGU | ACGUCCU | U | CUUCACC |
| 1547 | GGGUGAA | CUGAUGAG | X | CGAA | AAGGACG | CGUCCUU | C | UUCACCC |
| 1549 | UUGGGUG | CUGAUGAG | X | CGAA | AGAAGGA | UCCUUCU | U | CACCCAA |
| 1550 | CUUGGGU | CUGAUGAG | X | CGAA | AAGAAGG | CCUUCUU | C | ACCCAAG |
| 1574 | UGAGCUG | CUGAUGAG | X | CGAA | AUUCUCU | AGAGAAU | C | CAGCUCA |
| 1580 | UGUUUAU | CUGAUGAG | X | CGAA | AGCUGGA | UCCAGCU | C | AUAAACA |
| 1583 | UGGUGUU | CUGAUGAG | X | CGAA | AUGAGCU | AGCUCAU | A | AACACCA |
| 1607 | UCCUGUU | CUGAUGAG | X | CGAA | AUGUGCC | GGCACAU | C | AACAGGA |
| 1636 | GUUGAGG | CUGAUGAG | X | CGAA | AUCCAUG | AAUGAAU | C | CCUCAAC |
| 1640 | CGGUGUU | CUGAUGAG | X | CGAA | AGGGAUU | AAUCCCU | C | AACACCG |
| 1651 | GGCAAAG | CUGAUGAG | X | CGAA | ACCCGGU | ACCGGGU | U | CUUUGCC |
| 1652 | CGGCAAA | CUGAUGAG | X | CGAA | AACCCGG | CCGGGUU | C | UUUGCCG |
| 1654 | UGCGGCA | CUGAUGAG | X | CGAA | AGAACCC | GGGUUCU | U | UGCCGCA |
| 1655 | GUGCGGC | CUGAUGAG | X | CGAA | AAGAACC | GGUUCUU | U | GCCGCAC |
| 1666 | UGCGUAG | CUGAUGAG | X | CGAA | ACAGUGC | GCACUGU | U | CUACGCA |
| 1667 | GUGCGUA | CUGAUGAG | X | CGAA | AACAGUG | CACUGUU | C | UACGCAC |
| 1669 | GUGUGCG | CUGAUGAG | X | CGAA | AGAACAG | CUGUUCU | A | CGCACAC |
| 1681 | CGAGUUG | CUGAUGAG | X | CGAA | ACUUGUG | CACAAGU | U | CAACUCG |
| 1682 | ACGAGUU | CUGAUGAG | X | CGAA | AACUUGU | ACAAGUU | C | AACUCGU |
| 1687 | UCCGGAC | CUGAUGAG | X | CGAA | AGUUGAA | UUCAACU | C | GUCCGGA |
| 1690 | GCAUCCG | CUGAUGAG | X | CGAA | ACGAGUU | AACUCGU | C | CGGAUGC |
| 1723 | GUCGAUG | CUGAUGAG | X | CGAA | AGCUGCA | UGCAGCU | C | CAUCGAC |
| 1764 | GGCUCGG | CUGAUGAG | X | CGAA | AUAGGUG | CACCUAU | A | CCGAGCC |
| 1773 | AGGUCCC | CUGAUGAG | X | CGAA | AGGCUCG | CGAGCCU | A | GGGACCU |
| 1785 | GGCCUCU | CUGAUGAG | X | CGAA | AUCCAGG | CCUGGAU | C | AGAGGCC |
| 1794 | CAGCAGU | CUGAUGAG | X | CGAA | AGGCCUC | GAGGCCU | U | ACUGCUG |
| 1861 | GAAACAG | CUGAUGAG | X | CGAA | ACACUCG | CCAGUGU | A | CUGUUUC |
| 1866 | GGGGUGA | CUGAUGAG | X | CGAA | ACAGUAC | GUACUGU | U | UCACCCC |
| 1867 | UGGUGUG | CUGAUGAG | X | CGAA | AACAGUA | UACUGUU | U | CACCCCA |
| 1868 | UUGGGGU | CUGAUGAG | X | CGAA | AAACAGU | ACUGUUU | C | ACCCCAA |
| 1955 | UGUUGAG | CUGAUGAG | X | CGAA | AGCAGCA | UGCUGCU | U | CUCAACA |
| 1956 | UUGUUGA | CUGAUGAG | X | CGAA | AAGCAGC | GCUGCUU | C | UCAACAA |
| 1958 | UGUUGUU | CUGAUGAG | X | CGAA | AGAAGCA | UGCUUCU | C | AACAACA |
| 2020 | CUUGGUG | CUGAUGAG | X | CGAA | ACCCAGU | ACUGGGU | U | CACCAAG |
| 2021 | UCUUGGU | CUGAUGAG | X | CGAA | AACCCAG | CUGGGUU | C | ACCAAGA |
| 2094 | CGAAAGC | CUGAUGAG | X | CGAA | AUCCGUG | CACGGAU | U | GCUUUCG |
| 2098 | CUUCCGA | CUGAUGAG | X | CGAA | AGCAAUC | GAUUGCU | U | UCGGAAG |
| 2099 | GCUUCCG | CUGAUGAG | X | CGAA | AAGCAAU | AUUGCUU | U | CGGAAGC |
| 2100 | UGCUUCC | CUGAUGAG | X | CGAA | AAAGCAA | UUGCUUU | C | GGAAGCA |
| 2157 | AUACACC | CUGAUGAG | X | CGAA | AGGUGUU | AACACCU | A | GGUGUAU |
| 2163 | UCAACUA | CUGAUGAG | X | CGAA | ACACCUA | UAGGUGU | A | UAGUUGA |
| 2165 | AGUCAAC | CUGAUGAG | X | CGAA | AUACACC | GGUGUAU | A | GUUGACU |
| 2168 | GGUAGUC | CUGAUGAG | X | CGAA | ACUAUAC | GUAUAGU | U | GACUACC |
| 2173 | GUAUGGG | CUGAUGAG | X | CGAA | AGUCAAC | GUUGACU | A | CCCAUAC |
| 2179 | GAGCCUG | CUGAUGAG | X | CGAA | AUGGGUA | UACCCAU | A | CAGGCUC |
| 2186 | AGUGCCA | CUGAUGAG | X | CGAA | AGCCUGU | ACAGGCU | C | UGGCACU |
| 2194 | GCAGGGG | CUGAUGAG | X | CGAA | AGUGCCA | UGGCACU | A | CCCCUGC |
| 2207 | UAAAGUU | CUGAUGAG | X | CGAA | ACAGUGC | GCACUGU | C | AACUUUA |
| 2212 | GAUGGUA | CUGAUGAG | X | CGAA | AGUUGAC | GUCAACU | U | UACCAUC |
| 2213 | AGAUGGU | CUGAUGAG | X | CGAA | AAGUUGA | UCAACUU | U | ACCAUCU |
| 2214 | AAGAUGG | CUGAUGAG | X | CGAA | AAAGUUG | CAACUUU | A | CCAUCUU |
| 2222 | UAACCUU | CUGAUGAG | X | CGAA | AAGAUGG | CCAUCUU | U | AAGGUUA |
| 2223 | CUAACCU | CUGAUGAG | X | CGAA | AAAGAUG | CAUCUUU | A | AGGUUAG |
| 2228 | ACAUCCU | CUGAUGAG | X | CGAA | ACCUUAA | UUAAGGU | U | AGGAUGU |
| 2229 | UACAUCC | CUGAUGAG | X | CGAA | AACCUUA | UAAGGUU | A | GGAUGUA |
| 2236 | CCCCACA | CUGAUGAG | X | CGAA | ACAUCCU | AGGAUGU | A | UGUGGGG |
| 2283 | UCUCCUC | CUGAUGAG | X | CGAA | AGUCCAG | CUGGACU | C | GAGGAGA |
| 2366 | AACAGGG | CUGAUGAG | X | CGAA | AGUGUCU | AGACACU | U | CCCUGUU |
| 2367 | GAACAGG | CUGAUGAG | X | CGAA | AAGUGUC | GACACUU | C | CCUGUUC |
| 2373 | GUGAAGG | CUGAUGAG | X | CGAA | ACAGGGA | UCCCUGU | U | CCUUCAC |
| 2374 | GGUGAAG | CUGAUGAG | X | CGAA | AACAGGG | CCCUGUU | C | CUUCACC |
| 2377 | GGUGGUG | CUGAUGAG | X | CGAA | AGGAACA | UGUUCCU | U | CACCACC |
| 2378 | GGGUGGU | CUGAUGAG | X | CGAA | AAGGAAC | GUUCCUU | C | ACCACCC |
| 2387 | GAGCCGG | CUGAUGAG | X | CGAA | AGGGUGG | CCACCCU | A | CCGGCUC |
| 2394 | GUGGACA | CUGAUGAG | X | CGAA | AGCCGGU | ACCGGCU | C | UGUCCAC |
| 2398 | ACCAGUG | CUGAUGAG | X | CGAA | ACAGAGC | GCUCUGU | C | CACUGGU |
| 2406 | UGGAUCA | CUGAUGAG | X | CGAA | ACCAGUG | CACUGGU | U | UGAUCCA |
| 2407 | GUGGAUC | CUGAUGAG | X | CGAA | AACCAGU | ACUGGUU | U | GAUCCAC |
| 2411 | GGAGGUG | CUGAUGAG | X | CGAA | AUCAAAC | GUUUGAU | C | CACCUCC |
| 2443 | GUACAGG | CUGAUGAG | X | CGAA | ACUCCAC | GUGCAGU | A | CCUGUAC |
| 2449 | UAUACCG | CUGAUGAG | X | CGAA | ACAGGUA | UACCUGU | A | CGGUAUA |
| 2454 | GACCCUA | CUGAUGAG | X | CGAA | ACCGUAC | GUACGGU | A | UAGGGUC |
| 2456 | CUGACCC | CUGAUGAG | X | CGAA | AUACCGU | ACGGUAU | A | GGGUCAG |
| 2461 | AACCGCU | CUGAUGAG | X | CGAA | ACCCUAU | AUAGGGU | C | AGCGGUU |
| 2468 | AGGAGAC | CUGAUGAG | X | CGAA | ACCGCUG | CAGCGGU | U | GUCUCCU |
| 2471 | CAAAGGA | CUGAUGAG | X | CGAA | ACAACCG | CGGUUGU | C | UCCUUUG |
| 2473 | CACAAAG | CUGAUGAG | X | CGAA | AGACAAC | GUUGUCU | C | CUUUGUG |
| 2476 | GAUCACA | CUGAUGAG | X | CGAA | AGGAGAC | GUCUCCU | U | UGUGAUC |
| 2477 | UGAUCAC | CUGAUGAG | X | CGAA | AAGGAGA | UCUCCUU | U | GUGAUCA |
| 2483 | CCCAUUU | CUGAUGAG | X | CGAA | AUCACAA | UUGUGAU | C | AAAUGGG |
| 2494 | CACGAUA | CUGAUGAG | X | CGAA | ACUCCCA | UGGGAGU | A | UAUCGUG |
| 2496 | AACACGA | CUGAUGAG | X | CGAA | AUACUCC | GGAGUAU | A | UCGUGUU |
| 2498 | GCAACAC | CUGAUGAG | X | CGAA | AUAUACU | AGUAUAU | C | GUGUUGC |
| 2503 | GAAAAGC | CUGAUGAG | X | CGAA | ACACGAU | AUCGUGU | U | GCUUUUC |
| 2507 | GAAGGAA | CUGAUGAG | X | CGAA | AGCAACA | UGUUGCU | U | UUCCUUC |
| 2508 | AGAAGGA | CUGAUGAG | X | CGAA | AAGCAAC | GUUGCUU | U | UCCUUCU |
| 2509 | GAGAAGG | CUGAUGAG | X | CGAA | AAAGCAA | UUGCUUU | U | CCUUCUC |
| 2510 | GGAGAAG | CUGAUGAG | X | CGAA | AAAAGCA | UGCUUUU | C | CUUCUCC |
| 2513 | CCAGGAG | CUGAUGAG | X | CGAA | AGGAAAA | UUUUCCU | U | CUCCUGG |
| 2514 | GCCAGGA | CUGAUGAG | X | CGAA | AAGGAAA | UUUCCUU | C | UCCUGGC |
| 2516 | CCGCCAG | CUGAUGAG | X | CGAA | AGAAGGA | UCCUUCU | C | CUGGCGG |
| 2545 | CAUCCAC | CUGAUGAG | X | CGAA | AGCAGGC | GCCUGCU | U | GUGGAUG |
| 2564 | CCUGGGC | CUGAUGAG | X | CGAA | AUCAGCA | UGCUGAU | A | GCCCAGG |
| 2614 | GGCCAGG | CUGAUGAG | X | CGAA | ACGCCGC | GCGGCGU | C | CCUGGCC |
| 2636 | AGGAGAG | CUGAUGAG | X | CGAA | AUGCCAU | AUGGCAU | U | CUCUCCU |
| 2637 | AAGGAGA | CUGAUGAG | X | CGAA | AAUGCCA | UGGCAUU | C | UCUCCUU |
| 2639 | GGAAGGA | CUGAUGAG | X | CGAA | AGAAUGC | GCAUUCU | C | UCCUUCC |
| 2641 | AAGGAAG | CUGAUGAG | X | CGAA | AGAGAAU | AUUCUCU | C | CUUCCUU |
| 2644 | CACAAGG | CUGAUGAG | X | CGAA | AGGAGAG | CUCUCCU | U | CCUUGUG |
| 2645 | ACACAAG | CUGAUGAG | X | CGAA | AAGGAGA | UCUCCUU | C | CUUGUGU |
| 2648 | AAAACAC | CUGAUGAG | X | CGAA | AGGAAGG | CCUUCCU | U | GUGUUUU |
| 2653 | ACAGAAA | CUGAUGAG | X | CGAA | ACACAAG | CUUGUGU | U | UUUCUGU |
| 2654 | CACAGAA | CUGAUGAG | X | CGAA | AACACAA | UUGUGUU | U | UUCUGUG |
| 2655 | GCACAGA | CUGAUGAG | X | CGAA | AAACACA | UGUGUUU | U | UCUGUGC |
| 2656 | GGCACAG | CUGAUGAG | X | CGAA | AAAACAC | GUGUUUU | U | CUGUGCC |
| 2657 | CGGCACA | CUGAUGAG | X | CGAA | AAAAACA | UGUUUUU | C | UGUGCCG |
| 2732 | GGAGCAG | CUGAUGAG | X | CGAA | AGCAGCG | CGCUGCU | C | CUGCUCC |
| 2749 | UGGUGGU | CUGAUGAG | X | CGAA | ACGCCAG | CUGGCGU | U | ACCACCA |
| 2750 | GUGGUGG | CUGAUGAG | X | CGAA | AACGCCA | UGGCGUU | A | CCACCAC |
| 2791 | UCCACAC | CUGAUGAG | X | CGAA | AUGCAGC | GCUGCAU | C | GUGUGGA |
| 2807 | CUACAAA | CUGAUGAG | X | CGAA | ACCACCC | GGGUGGU | U | UUUGUAG |
| 2808 | CCUACAA | CUGAUGAG | X | CGAA | AACCACC | GGUGGUU | U | UUGUAGG |
| 2809 | ACCUACA | CUGAUGAG | X | CGAA | AAACCAC | GUGGUUU | U | UGUAGGU |
| 2810 | GACCUAC | CUGAUGAG | X | CGAA | AAAACCA | UGGUUUU | U | GUAGGUC |
| 2813 | UUAGACC | CUGAUGAG | X | CGAA | ACAAAAA | UUUUUGU | A | GGUCUAA |
| 2817 | AGUAUUA | CUGAUGAG | X | CGAA | ACCUACA | UGUAGGU | C | UAAUACU |
| 2819 | AGAGUAU | CUGAUGAG | X | CGAA | AGACCUA | UAGGUCU | A | AUACUCU |
| 2822 | UCAAGAG | CUGAUGAG | X | CGAA | AUUAGAC | GUCUAAU | A | CUCUUGA |
| 2825 | AGGUCAA | CUGAUGAG | X | CGAA | AGUAUUA | UAAUACU | C | UUGACCU |
| 2827 | CAAGGUC | CUGAUGAG | X | CGAA | AGAGUAU | AUACUCU | U | GACCUUG |
| 2833 | UGGUGAC | CUGAUGAG | X | CGAA | AGGUCAA | UUGACCU | U | GUCACCA |
| 2836 | GUGUGGU | CUGAUGAG | X | CGAA | ACAAGGU | ACCUUGU | C | ACCACAC |
| 2845 | CACUUUG | CUGAUGAG | X | CGAA | AGUGUGG | CCACACU | A | CAAAGUG |
| 2854 | GGCGAGG | CUGAUGAG | X | CGAA | ACACUUU | AAAGUGU | U | CCUCGCC |
| 2855 | UGGCGAG | CUGAUGAG | X | CGAA | AACACUU | AAGUGUU | C | CUCGCCA |
| 2858 | GCCUGGC | CUGAUGAG | X | CGAA | AGGAACA | UGUUCCU | C | GCCAGGC |
| 2867 | ACCAUAU | CUGAUGAG | X | CGAA | AGCCUGG | CCAGGCU | C | AUAUGGU |
| 2870 | ACCACCA | CUGAUGAG | X | CGAA | AUGAGCC | GGCUCAU | A | UGGUGGU |
| 2889 | CUGGUGA | CUGAUGAG | X | CGAA | AAAGUAU | AUACUUU | A | UCACCAG |
| 2891 | CCCUGGU | CUGAUGAG | X | CGAA | AUAAAGU | ACUUUAU | C | ACCAGGG |
| 2993 | CAAAGAU | CUGAUGAG | X | CGAA | AGCUCUG | CAGAGCU | A | AUCUUUG |
| 2996 | UGUCAAA | CUGAUGAG | X | CGAA | AUUAGCU | AGCUAAU | C | UUUGACA |
| 2998 | AAUGUCA | CUGAUGAG | X | CGAA | AGAUUAG | CUAAUCU | U | UGACAUU |
| 2999 | UAAUGUC | CUGAUGAG | X | CGAA | AAGAUUA | UAAUCUU | U | GACAUUA |
| 3005 | GUUUGGU | CUGAUGAG | X | CGAA | AUGUCAA | UUGACAU | U | ACCAAAC |
| 3006 | AGUUUGG | CUGAUGAG | X | CGAA | AAUGUCA | UGACAUU | A | CCAAACU |
| 3014 | CGAGCAG | CUGAUGAG | X | CGAA | AGUUUGG | CCAAACU | C | CUGCUCG |
| 3020 | GAAUGGC | CUGAUGAG | X | CGAA | AGCAGGA | UCCUGCU | C | GCCAUUC |
| 3026 | GACCGAG | CUGAUGAG | X | CGAA | AUGGCGA | UCGCCAU | U | CUCGGUC |
| 3027 | GGACCGA | CUGAUGAG | X | CGAA | AAUGGCG | CGCCAUU | C | UCGGUCC |
| 3029 | GCGGACC | CUGAUGAG | X | CGAA | AGAAUGG | CCAUUCU | C | GGUCCGC |
| 3033 | AUGAGCG | CUGAUGAG | X | CGAA | ACCGAGA | UCUCGGU | C | CGCUCAU |
| 3038 | GCACCAU | CUGAUGAG | X | CGAA | AGCGGAC | GUCCGCU | C | AUGGUGC |
| 3047 | CAGCCUG | CUGAUGAG | X | CGAA | AGCACCA | UGGUGCU | C | CAGGCUG |
| 3073 | UACAAAG | CUGAUGAG | X | CGAA | ACGGCAU | AUGCCGU | A | CUUUGUA |
| 3076 | GCGUACA | CUGAUGAG | X | CGAA | AGUACGG | CCGUACU | U | UGUACGC |
| 3077 | CGCGUAC | CUGAUGAG | X | CGAA | AAGUACG | CGUACUU | U | GUACGCG |
| 3080 | GAGCGCG | CUGAUGAG | X | CGAA | ACAAAGU | ACUUUGU | A | CGCGCUC |
| 3087 | AGCCCCU | CUGAUGAG | X | CGAA | AGCGCGU | ACGCGCU | C | AGGGGCU |
| 3095 | CACGAAU | CUGAUGAG | X | CGAA | AGCCCCU | AGGGGCU | U | AUUCGUG |
| 3096 | GCACGAA | CUGAUGAG | X | CGAA | AAGCCCC | GGGGCUU | A | UUCGUGC |
| 3098 | AUGCACG | CUGAUGAG | X | CGAA | AUAAGCC | GGCUUAU | U | CGUGCAU |
| 3099 | CAUGCAC | CUGAUGAG | X | CGAA | AAUAAGC | GCUUAUU | C | GUGCAUG |
| 3112 | CCGCACC | CUGAUGAG | X | CGAA | ACAUGCA | UGCAUGU | U | GGUGCGG |
| 3125 | CUCCGGC | CUGAUGAG | X | CGAA | ACUUUCC | GGAAAGU | A | GCCGGAG |
| 3180 | ACGUACG | CUGAUGAG | X | CGAA | ACCUGUC | GACAGGU | A | CGUACGU |
| 3184 | AUAGACG | CUGAUGAG | X | CGAA | ACGUACC | GGUACGU | A | CGUCUAU |
| 3188 | GGUCAUA | CUGAUGAG | X | CGAA | ACGUACG | CGUACGU | C | UAUGACC |
| 3190 | AUGGUCA | CUGAUGAG | X | CGAA | AGACGUA | UACGUCU | A | UGACCAU |
| 3198 | GGGGUAA | CUGAUGAG | X | CGAA | AUGGUCA | UGACCAU | C | UUACCCC |
| 3200 | GCGGGGU | CUGAUGAG | X | CGAA | AGAUGGU | ACCAUCU | U | ACCCCGC |
| 3201 | AGCGGGG | CUGAUGAG | X | CGAA | AAGAUGG | CCAUCUU | A | CCCCGCU |
| 3254 | CGGGCUC | CUGAUGAG | X | CGAA | ACUGCCA | UGGCAGU | A | GAGCCCG |
| 3269 | UGUCAGA | CUGAUGAG | X | CGAA | AAGACGA | UCGUCUU | C | UCUGACA |
| 3271 | CAUGUCA | CUGAUGAG | X | CGAA | AGAAGAC | GUCUUCU | C | UGACAUG |
| 3374 | GUCCCAG | CUGAUGAG | X | CGAA | AGUAUCU | AGAUACU | U | CUGGGAC |
| 3375 | GGUCCCA | CUGAUGAG | X | CGAA | AAGUAUC | GAUACUU | C | UGGGACC |
| 3390 | UCAAUGC | CUGAUGAG | X | CGAA | AUCGGCC | GGCCGAU | A | GCAUUGA |
| 3395 | GCCCUUC | CUGAUGAG | X | CGAA | AUGCUAU | AUAGCAU | U | GAAGGGC |
| 3436 | UUGGGCG | CUGAUGAG | X | CGAA | AGGCCGU | ACGGCCU | A | CGCCCAA |
| 3458 | AACCAAG | CUGAUGAG | X | CGAA | AGGCCCC | GGGGCCU | A | CUUGGUU |
| 3461 | UGCAACC | CUGAUGAG | X | CGAA | AGUAGGC | GCCUACU | U | GGUUGCA |
| 3465 | ACAAUGC | CUGAUGAG | X | CGAA | ACCAAGU | ACUUGGU | U | GCAUUGU |
| 3470 | UAGUAAC | CUGAUGAG | X | CGAA | AUGCAAC | GUUGCAU | U | GUUACUA |
| 3473 | GGCUAGU | CUGAUGAG | X | CGAA | ACAAUGC | GCAUUGU | U | ACUAGCC |
| 3474 | AGGCUAG | CUGAUGAG | X | CGAA | AACAAUG | CAUUGUU | A | CUAGCCU |
| 3477 | GUGAGGC | CUGAUGAG | X | CGAA | AGUAACA | UGUUACU | A | GCCUCAC |
| 3506 | CCCCUUC | CUGAUGAG | X | CGAA | ACCUGGU | ACCAGGU | C | GAAGGGG |
| 3544 | CAGGAAA | CUGAUGAG | X | CGAA | AUUGUGU | ACACAAU | C | UUUCCUG |
| 3546 | GCCAGGA | CUGAUGAG | X | CGAA | AGAUUGU | ACAAUCU | U | UCCUGGC |
| 3547 | CGCCAGG | CUGAUGAG | X | CGAA | AAGAUUG | CAAUCUU | U | CCUGGCG |
| 3548 | UCGCCAG | CUGAUGAG | X | CGAA | AAAGAUU | AAUCUUU | C | CUGGCGA |
| 3563 | CACCAUU | CUGAUGAG | X | CGAA | ACGCAGG | CCUGCGU | U | AAUGGUG |
| 3564 | ACACCAU | CUGAUGAG | X | CGAA | AACGCAG | CUGCGUU | A | AUGGUGU |
| 3584 | CGUGGAA | CUGAUGAG | X | CGAA | ACGGUCC | GGACCGU | C | UUCCACG |
| 3586 | GCCGUGG | CUGAUGAG | X | CGAA | AGACGGU | ACCGUCU | U | CCACGGC |
| 3587 | CGCCGUG | CUGAUGAG | X | CGAA | AAGACGG | CCGUCUU | C | CACGGCG |
| 3632 | UUUGGGU | CUGAUGAG | X | CGAA | AUUGGGC | GCCCAAU | C | ACCCAAA |
| 3643 | AUUAGUG | CUGAUGAG | X | CGAA | ACAUUUG | CAAAUGU | A | CACUAAU |
| 3648 | UCUACAU | CUGAUGAG | X | CGAA | AGUGUAC | GUACACU | A | AUGUAGA |
| 3653 | CUUGGUC | CUGAUGAG | X | CGAA | ACAUUAG | CUAAUGU | A | GACCAAG |
| 3665 | AGCCGAC | CUGAUGAG | X | CGAA | AGGUCUU | AAGACCU | C | GUCGGCU |
| 3668 | GCCAGCC | CUGAUGAG | X | CGAA | ACGAGGU | ACCUCGU | C | GGCUGGC |
| 3720 | UCCGAGC | CUGAUGAG | X | CGAA | ACCGCAG | CUGCGGU | A | GCUCGGA |
| 3758 | CCGGAAU | CUGAUGAG | X | CGAA | ACGUCAG | CUGACGU | C | AUUCCGG |
| 3815 | AAUAGGA | CUGAUGAG | X | CGAA | ACGGGUC | GACCCGU | C | UCCUAUU |
| 3817 | CAAAUAG | CUGAUGAG | X | CGAA | AGACGGG | CCCGUCU | C | CUAUUUG |
| 3820 | CUUCAAA | CUGAUGAG | X | CGAA | AGGAGAC | GUCUCCU | A | UUUGAAG |
| 3822 | CCCUUCA | CUGAUGAG | X | CGAA | AUAGGAG | CUCCUAU | U | UGAAGGG |
| 3823 | GCCCUUC | CUGAUGAG | X | CGAA | AAUAGGA | UCCUAUU | U | GAAGGGC |
| 3832 | ACCCGAA | CUGAUGAG | X | CGAA | AGCCCUU | AAGGGCU | C | UUCGGGU |
| 3834 | CCACCCG | CUGAUGAG | X | CGAA | AGAGCCC | GGGCUCU | U | CGGGUGG |
| 3925 | GGGUAUG | CUGAUGAG | X | CGAA | AGUCCAC | GUGGACU | U | CAUACCC |
| 3926 | CGGGUAU | CUGAUGAG | X | CGAA | AAGUCCA | UGGACUU | C | AUACCCG |
| 3929 | CAACGGG | CUGAUGAG | X | CGAA | AUGAAGU | ACUUCAU | A | CCCGUUG |
| 3935 | UAGACUC | CUGAUGAG | X | CGAA | ACGGGUA | UACCCGU | U | GAGUCUA |
| 3940 | UUCCAUA | CUGAUGAG | X | CGAA | ACUCAAC | GUUGAGU | C | UAUGGAA |
| 3942 | GUUUCCA | CUGAUGAG | X | CGAA | AGACUCA | UGAGUCU | A | UGGAAAC |
| 3951 | CGCAUAG | CUGAUGAG | X | CGAA | AGUUUCC | GGAAACU | A | CUAUGCG |
| 3954 | GACCGCA | CUGAUGAG | X | CGAA | AGUAGUU | AACUACU | A | UGCGGUC |
| 3961 | GACCGGG | CUGAUGAG | X | CGAA | ACCGCAU | AUGCGGU | C | CCCGGUC |
| 3968 | CCGUGAA | CUGAUGAG | X | CGAA | ACCGGGG | CCCCGGU | C | UUCACGG |
| 3970 | GUCCGUG | CUGAUGAG | X | CGAA | AGACCGG | CCGGUCU | U | CACGGAC |
| 3971 | UGUCCGU | CUGAUGAG | X | CGAA | AAGACCG | CGGUCUU | C | ACGGACA |
| 3982 | GGGAGAU | CUGAUGAG | X | CGAA | AGUUGUC | GACAACU | C | AUCUCCC |
| 3985 | CGGGGGA | CUGAUGAG | X | CGAA | AUGAGUU | AACUCAU | C | UCCCCCG |
| 3987 | GCCGGGG | CUGAUGAG | X | CGAA | AGAUGAG | CUCAUCU | C | CCCCGGC |
| 3998 | UCUGCGG | CUGAUGAG | X | CGAA | ACGGCCG | CGGCCGU | A | CCGCAGA |
| 4009 | CACUUGG | CUGAUGAG | X | CGAA | AUGUCUG | CAGACAU | U | CCAAGUG |
| 4010 | CCACUUG | CUGAUGAG | X | CGAA | AAUGUCU | AGACAUU | C | CAAGUGG |
| 4023 | GCGUGUA | CUGAUGAG | X | CGAA | AUGGGCC | GGCCCAU | C | UACACGC |
| 4025 | GAGCGUG | CUGAUGAG | X | CGAA | AGAUGGG | CCCAUCU | A | CACGCUC |
| 4032 | CCAGUGG | CUGAUGAG | X | CGAA | AGCGUGU | ACACGCU | C | CCACUGG |
| 4094 | GGACGAG | CUGAUGAG | X | CGAA | ACCUUGU | ACAAGGU | A | CUCGUCC |
| 4097 | UCAGGAC | CUGAUGAG | X | CGAA | AGUACCU | AGGUACU | C | GUCCUGA |
| 4100 | GGUUCAG | CUGAUGAG | X | CGAA | ACGAGUA | UACUCGU | C | CUGAACC |
| 4111 | GGCAACA | CUGAUGAG | X | CGAA | AUGGGUU | AACCCAU | C | UGUUGCC |
| 4126 | AAAACCC | CUGAUGAG | X | CGAA | AGGUGGC | GCCACCU | U | GGGUUUU |
| 4131 | GCCCCAA | CUGAUGAG | X | CGAA | ACCCAAG | CUUGGGU | U | UUGGGGC |
| 4132 | CGCCCCA | CUGAUGAG | X | CGAA | AACCCAA | UUGGGUU | U | UGGGGCG |
| 4133 | ACGCCCC | CUGAUGAG | X | CGAA | AAACCCA | UGGGUUU | U | GGGGCGU |
| 4141 | AGACAUA | CUGAUGAG | X | CGAA | ACGCCCC | GGGGCGU | A | UAUGUCU |
| 4143 | UUAGACA | CUGAUGAG | X | CGAA | AUACGCC | GGCGUAU | A | UGUCUAA |
| 4147 | UGCCUUA | CUGAUGAG | X | CGAA | ACAUAUA | UAUAUGU | C | UAAGGCA |
| 4149 | UGUGCCU | CUGAUGAG | X | CGAA | AGACAUA | UAUGUCU | A | AGGCACA |
| 4161 | GGGUCGG | CUGAUGAG | X | CGAA | ACCAUGU | ACAUGGU | A | CCGACCC |
| 4196 | CCGUGGU | CUGAUGAG | X | CGAA | AUGGUCC | GGACCAU | U | ACCACGG |
| 4197 | CCCGUGG | CUGAUGAG | X | CGAA | AAUGGUC | GACCAUU | A | CCACGGG |
| 4214 | AGUACGU | CUGAUGAG | X | CGAA | AUGGGGG | CCCCCAU | C | ACGUACU |
| 4219 | GGUGGAG | CUGAUGAG | X | CGAA | ACGUGAU | AUCACGU | A | CUCCACC |
| 4222 | AUAGGUG | CUGAUGAG | X | CGAA | AGUACGU | ACGUACU | C | CACCUAU |
| 4257 | CCCCCAG | CUGAUGAG | X | CGAA | ACAUCCA | UGGAUGU | U | CUGGGGG |
| 4258 | GCCCCCA | CUGAUGAG | X | CGAA | AACAUCC | GGAUGUU | C | UGGGGGC |
| 4270 | GAUAUCA | CUGAUGAG | X | CGAA | AGGCGCC | GGCGCCU | A | UGAUAUC |
| 4275 | AUUAUGA | CUGAUGAG | X | CGAA | AUCAUAG | CUAUGAU | A | UCAUAAU |
| 4277 | AUAUUAU | CUGAUGAG | X | CGAA | AUAUCAU | AUGAUAU | C | AUAAUAU |
| 4300 | GUCAGUU | CUGAUGAG | X | CGAA | AGUGGCA | UGCCACU | C | AACUGAC |
| 4309 | GGUAGUC | CUGAUGAG | X | CGAA | AGUCAGU | ACUGACU | C | GACUACC |
| 4314 | AGGAUGG | CUGAUGAG | X | CGAA | AGUCGAG | CUCGACU | A | CCAUCCU |
| 4319 | UGCCCAG | CUGAUGAG | X | CGAA | AUGGUAG | CUACCAU | C | CUGGGCA |
| 4328 | CUGUGCC | CUGAUGAG | X | CGAA | AUGCCCA | UGGGCAU | C | GGCACAG |
| 4389 | GGAGGCG | CUGAUGAG | X | CGAA | AGCGGUG | CACCGCU | A | CGCCUCC |
| 4395 | GAUCCCG | CUGAUGAG | X | CGAA | AGGCGUA | UACGCCU | C | CGGGAUC |
| 4402 | GGUAACC | CUGAUGAG | X | CGAA | AUCCCGG | CCGGGAU | C | GGUUACC |
| 4406 | GCACGGU | CUGAUGAG | X | CGAA | ACCGAUC | GAUCGGU | U | ACCGUGC |
| 4407 | GGCACGG | CUGAUGAG | X | CGAA | AACCGAU | AUCGGUU | A | CCGUGCC |
| 4427 | CCUCCUC | CUGAUGAG | X | CGAA | AUAUUUG | CAAAUAU | U | GAGGAGC |
| 4440 | UUGGACA | CUGAUGAG | X | CGAA | AGCCACC | GGUGGCU | C | UGUCCAA |
| 4465 | GCCAUAG | CUGAUGAG | X | CGAA | AGGGGAU | AUCCCCU | U | CUAUGGC |
| 4466 | UGCCAUA | CUGAUGAG | X | CGAA | AAGGGGA | UCCCCUU | C | UAUGGCA |
| 4468 | CUUGCCA | CUGAUGAG | X | CGAA | AGAAGGG | CCCUUCU | A | UGGCAAG |
| 4512 | AAAAUGA | CUGAUGAG | X | CGAA | AUGCCUU | AAGGCAU | C | UCAUUUU |
| 4514 | AGAAAAU | CUGAUGAG | X | CGAA | AGAUGCC | GGCAUCU | C | AUUUUCU |
| 4517 | GGCAGAA | CUGAUGAG | X | CGAA | AUGAGAU | AUCUCAU | U | UUCUGCC |
| 4518 | UGGCAGA | CUGAUGAG | X | CGAA | AAUGAGA | UCUCAUU | U | UCUGCCA |
| 4519 | GUGGCAG | CUGAUGAG | X | CGAA | AAAUGAG | CUCAUUU | U | CUGCCAC |
| 4520 | AGUGGCA | CUGAUGAG | X | CGAA | AAAAUGA | UCAUUUU | C | UGCCACU |
| 4550 | UUGCGGC | CUGAUGAG | X | CGAA | AGCUCAU | AUGAGCU | C | GCCGCAA |
| 4564 | GAGGCCU | CUGAUGAG | X | CGAA | ACAGCUU | AAGCUGU | C | AGGCCUC |
| 4571 | UGAUUCC | CUGAUGAG | X | CGAA | AGGCCUG | CAGGCCU | C | GGAAUCA |
| 4602 | ACGUCAA | CUGAUGAG | X | CGAA | ACCCCGG | CCGGGGU | C | UUGACGU |
| 4604 | ACACGUC | CUGAUGAG | X | CGAA | AGACCCC | GGGGUCU | U | GACGUGU |
| 4612 | UAUGACG | CUGAUGAG | X | CGAA | ACACCUC | GACGUGU | C | CGUCAUA |
| 4637 | CGAUAAC | CUGAUGAG | X | CGAA | ACAUCUC | CAGAUGU | C | GUUAUCG |
| 4640 | CCACGAU | CUGAUGAG | X | CGAA | ACGACAU | AUGUCGU | U | AUCGUGG |
| 4641 | GCCACGA | CUGAUGAG | X | CGAA | AACGACA | UGUCGUU | A | UCGUGGC |
| 4643 | UUGCCAC | CUGAUGAG | X | CGAA | AUAACGA | UCGUUAU | C | GUGGCAA |
| 4659 | GUCAUUA | CUGAUGAG | X | CGAA | AGCGUCU | AGACGCU | C | UAAUGAC |
| 4661 | CCGUCAU | CUGAUGAG | X | CGAA | AGAGCGU | ACGCUCU | A | AUGACGG |
| 4684 | CGAGUCA | CUGAUGAG | X | CGAA | AGUCACC | GGUGACU | U | UGACUCG |
| 4685 | CCGAGUC | CUGAUGAG | X | CGAA | AAGUCAC | GUGACUU | U | GACUCGG |
| 4690 | GAUCACC | CUGAUGAG | X | CGAA | AGUCAAA | UUUGACU | C | GGUGAUC |
| 4715 | UCUGGGU | CUGAUGAG | X | CGAA | ACACAUG | CAUGUGU | C | ACCCAGA |
| 4727 | UGAAAUC | CUGAUGAG | X | CGAA | ACUGUCU | AGACAGU | C | GAUUUCA |
| 4731 | AAGCUGA | CUGAUGAG | X | CGAA | AUCGACU | AGUCGAU | U | UCAGCUU |
| 4732 | CAAGCUG | CUGAUGAG | X | CGAA | AAUCGAC | GUCGAUU | U | CAGCUUG |
| 4733 | CCAAGCU | CUGAUGAG | X | CGAA | AAAUCGA | UCGAUUU | C | AGCUUGG |
| 4738 | GGGAUCC | CUGAUGAG | X | CGAA | AGCUGAA | UUCAGCU | U | GGAUCCC |
| 4743 | AAGGUGG | CUGAUGAG | X | CGAA | AUCCAAG | CUUGGAU | C | CCACCUU |
| 4750 | AAUGGUA | CUGAUGAG | X | CGAA | AGGUGGG | CCCACCU | U | UACCAUU |
| 4751 | CAAUGGU | CUGAUGAG | X | CGAA | AAGGUGG | CCACCUU | U | ACCAUUG |
| 4752 | UCAAUGG | CUGAUGAG | X | CGAA | AAAGGUG | CACCUUU | A | CCAUUGA |
| 4757 | UCCUCUC | CUGAUGAG | X | CGAA | AUGGUAA | UUACCAU | U | GAGACGA |
| 4824 | CCUCCCC | CUGAUGAG | X | CGAA | ACCCCUG | CAGGGGU | A | GGGGAGG |
| 4835 | ACCUGUA | CUGAUGAG | X | CGAA | AUGCCUC | GAGGCAU | C | UACAGGU |
| 4837 | AAACCUG | CUGAUGAG | X | CGAA | AGAUGCC | GGCAUCU | A | CAGGUUU |
| 4843 | AGUCACA | CUGAUGAG | X | CGAA | ACCUGUA | UACAGGU | U | UGUGACU |
| 4844 | GAGUCAC | CUGAUGAG | X | CGAA | AACCUGU | ACAGGUU | U | GUGACUC |
| 4851 | UCUCCCG | CUGAUGAG | X | CGAA | AGUCACA | UGUGACU | C | CGGGAGA |
| 4867 | CAUGCCC | CUGAUGAG | X | CGAA | AGGGCCG | CGGCCCU | C | GGGCAUG |
| 4876 | AGAAUCG | CUGAUGAG | X | CGAA | ACAUGCC | GGCAUGU | U | CGAUUCU |
| 4877 | AAGAAUC | CUGAUGAG | X | CGAA | AACAUGC | GCAUGUU | C | GAUUCUU |
| 4881 | ACCGAAG | CUGAUGAG | X | CGAA | AUCGAAC | GUUCGAU | U | CUUCGGU |
| 4882 | GACCGAA | CUGAUGAG | X | CGAA | AAUCGAA | UUCGAUU | C | UUCGGUC |
| 4884 | AGGACCG | CUGAUGAG | X | CGAA | AGAAUCG | CGAUUCU | U | CGGUCCU |
| 4885 | CAGGACC | CUGAUGAG | X | CGAA | AAGAAUC | GAUUCUU | C | GGUCCUG |
| 4889 | CACACAG | CUGAUGAG | X | CGAA | ACCGAAG | CUUCGGU | C | CUGUGUG |
| 4903 | CGCGUCA | CUGAUGAG | X | CGAA | AGCACUC | GAGUGCU | A | UGACGCG |
| 5011 | UUCCCAG | CUGAUGAG | X | CGAA | ACUCCAG | CUGGAGU | U | CUGGGAA |
| 5012 | UUUCCCA | CUGAUGAG | X | CGAA | AACUCCA | UGGAGUU | C | UGGGAAA |
| 5024 | CUGUGAA | CUGAUGAG | X | CGAA | ACGCUUU | AAAGCGU | C | UUCACAG |
| 5026 | GCCUGUG | CUGAUGAG | X | CGAA | AGACGCU | AGCGUCU | U | CACAGGC |
| 5027 | GGCCUGU | CUGAUGAG | X | CGAA | AAGACGC | GCGUCUU | C | ACAGGCC |
| 5036 | UGUGGGU | CUGAUGAG | X | CGAA | AGGCCUG | CAGGCCU | C | ACCCACA |
| 5045 | GGGCAUC | CUGAUGAG | X | CGAA | AUGUGGG | CCCACAU | A | GAUGCCC |
| 5056 | GGACAGG | CUGAUGAG | X | CGAA | AGUGGGC | GCCCACU | U | CCUGUCC |
| 5057 | GGGACAG | CUGAUGAG | X | CGAA | AAGUGGG | CCCACUU | C | CUGUCCC |
| 5062 | GGUUUGG | CUGAUGAG | X | CGAA | ACAGGAA | UUCCUGU | C | CCAAACC |
| 5089 | GUAAGGG | CUGAUGAG | X | CGAA | AGUUGUC | GACAACU | U | CCCUUAC |
| 5090 | GGUAAGG | CUGAUGAG | X | CGAA | AAGUUGU | ACAACUU | C | CCUUACC |
| 5094 | ACCAGGU | CUGAUGAG | X | CGAA | AGGGAAG | CUUCCCU | U | ACCUGGU |
| 5095 | UACCAGG | CUGAUGAG | X | CGAA | AAGGGAA | UUCCCUU | A | CCUGGUA |
| 5139 | GGAGGUG | CUGAUGAG | X | CGAA | AGCCUGA | UCAGGCU | C | CACCUCC |
| 5145 | CACGAUG | CUGAUGAG | X | CGAA | AGGUGGA | UCCACCU | C | CAUCGUG |
| 5149 | AUCCCAC | CUGAUGAG | X | CGAA | AUGGAGG | CCUCCAU | C | GUGGGAU |
| 5157 | CACAUUU | CUGAUGAG | X | CGAA | AUCCCAC | GUGGGAU | C | AAAUGUG |
| 5172 | CGUAUGA | CUGAUGAG | X | CGAA | ACACUUC | GAAGUGU | C | UCAUACG |
| 5174 | GCCGUAU | CUGAUGAG | X | CGAA | AGACACU | AGUGUCU | C | AUACGGC |
| 5177 | UAAGCCG | CUGAUGAG | X | CGAA | AUGAGAC | GUCUCAU | A | CGGCUUA |
| 5183 | UAGGUUU | CUGAUGAG | X | CGAA | AGCCGUA | UACGGCU | U | AAACCUA |
| 5184 | GUAGGUU | CUGAUGAG | X | CGAA | AAGCCGU | ACGGCUU | A | AACCUAC |
| 5190 | UGCAGCG | CUGAUGAG | X | CGAA | AGGUUUA | UAAACCU | A | CGCUGCA |
| 5225 | CGGCUCC | CUGAUGAG | X | CGAA | AGCCUAU | AUAGGCU | A | GGAGCCG |
| 5234 | CAUUUUG | CUGAUGAG | X | CGAA | ACGGCUC | GAGCCGU | U | CAAAAUG |
| 5235 | UCAUUUU | CUGAUGAG | X | CGAA | AACGGCU | AGCCGUU | C | AAAAUGA |
| 5246 | UGAGGGU | CUGAUGAG | X | CGAA | AUCUCAU | AUGAGAU | C | ACCCUCA |
| 5252 | GAUGUGU | CUGAUGAG | X | CGAA | AGGGUGA | UCACCCU | C | ACACAUC |
| 5259 | GUUAUGG | CUGAUGAG | X | CGAA | AUGUGUG | CACACAU | C | CCAUAAC |
| 5264 | AUUUGGU | CUGAUGAG | X | CGAA | AUGGGAU | AUCCCAU | A | ACCAAAU |
| 5272 | CAUGAUG | CUGAUGAG | X | CGAA | AUUUGGU | ACCAAAU | U | CAUCAUG |
| 5273 | CCAUGAU | CUGAUGAG | X | CGAA | AAUUUGG | CCAAAUU | C | AUCAUGG |
| 5276 | AUGCCAU | CUGAUGAG | X | CGAA | AUGAAUU | AAUUCAU | C | AUGGCAU |
| 5290 | GUCGGCC | CUGAUGAG | X | CGAA | ACAUGCA | UGCAUGU | C | GGCCGAC |
| 5349 | GCGGCCA | CUGAUGAG | X | CGAA | AGCUGCA | UGCAGCU | C | UGGCCGC |
| 5384 | CCACAAU | CUGAUGAG | X | CGAA | ACCACAC | GUGUGGU | C | AUUGUGG |
| 5387 | UACCCAC | CUGAUGAG | X | CGAA | AUGACCA | UGGUCAU | U | GUGGGUA |
| 5394 | AUGAUCC | CUGAUGAG | X | CGAA | ACCCACA | UGUGGGU | A | GGAUCAU |
| 5402 | CGGACAA | CUGAUGAG | X | CGAA | AUGAUCC | GGAUCAU | U | UUGUCCG |
| 5403 | CCGGACA | CUGAUGAG | X | CGAA | AAUGAUC | GAUCAUU | U | UGUCCGG |
| 5404 | CCCGGAC | CUGAUGAG | X | CGAA | AAAUGAU | AUCAUUU | U | GUCCGGG |
| 5407 | CCUCCCG | CUGAUGAG | X | CGAA | ACAAAAU | AUUUUGU | C | CGGGAGG |
| 5441 | GGUAGAG | CUGAUGAG | X | CGAA | ACUUCCC | GGGAAGU | C | CUCUACC |
| 5444 | CCCGGUA | CUGAUGAG | X | CGAA | AGGACUG | AAGUCCU | C | UACCGGG |
| 5446 | CUCCCGG | CUGAUGAG | X | CGAA | AGAGGAC | GUCCUCU | A | CCGGGAG |
| 5455 | UUCAUCG | CUGAUGAG | X | CGAA | ACUCCCG | CGGGAGU | U | CGAUGAA |
| 5456 | UUUCAUC | CUGAUGAG | X | CGAA | AACUCCC | GGGAGUU | C | GAUGAAA |
| 5479 | GAGGUCG | CUGAUGAG | X | CGAA | AGGCGCA | UGCGCCU | C | ACACCUC |
| 5486 | UGUAAGG | CUGAUGAG | X | CGAA | AGGUGUG | CACACCU | C | CCUUACA |
| 5490 | UCGAUGU | CUGAUGAG | X | CGAA | AGGGAGG | CCUCCCU | U | ACAUCGA |
| 5491 | UUCGAUG | CUGAUGAG | X | CGAA | AAGGGAG | CUCCCUU | A | CAUCGAA |
| 5495 | CCUGUUC | CUGAUGAG | X | CGAA | AUGUAAG | CUUACAU | C | GAACAGG |
| 5513 | GCUCGGC | CUGAUGAG | X | CGAA | AGCUGCA | UGCAGCU | C | GCCGAGC |
| 5540 | GCAACCC | CUGAUGAG | X | CGAA | AGUGCCU | AGGCACU | C | GGGUUGC |
| 5545 | UUGCAGC | CUGAUGAG | X | CGAA | ACCCGAG | CUCGGGU | U | GCUGCAA |
| 5644 | GCUGAUG | CUGAUGAG | X | CGAA | AGUUCCA | UGGAACU | U | CAUCAGC |
| 5645 | CGCUGAU | CUGAUGAG | X | CGAA | AAGUUCC | GGAACUU | C | AUCAGCG |
| 5648 | UCCCGCU | CUGAUGAG | X | CGAA | AUGAAGU | ACUUCAU | C | AGCGGGA |
| 5657 | AAUACUG | CUGAUGAG | X | CGAA | AUCCCGC | GCGGGAU | A | CAGUAUU |
| 5662 | UGCUAAA | CUGAUGAG | X | CGAA | ACUGUAU | AUACAGU | A | UUUAGCA |
| 5664 | CCUGCUA | CUGAUGAG | X | CGAA | AUACUGU | ACAGUAU | U | UAGCAGG |
| 5665 | GCCUGCU | CUGAUGAG | X | CGAA | AAUACUG | CAGUAUU | U | AGCAGGC |
| 5666 | AGCCUGC | CUGAUGAG | X | CGAA | AAAUACU | AGUAUUU | A | GCAGGCU |
| 5677 | CAGAGUG | CUGAUGAG | X | CGAA | AUAAGCC | GGCUUAU | C | CACUCUG |
| 5682 | CCAGGCA | CUGAUGAG | X | CGAA | AGUGGAU | AUCCACU | C | UGCCUGG |
| 5702 | GUGAUGC | CUGAUGAG | X | CGAA | AUCGCGG | CCGCGAU | A | GCAUCAC |
| 5707 | CAUCAGU | CUGAUGAG | X | CGAA | AUGCUAU | AUAGCAU | C | ACUGAUG |
| 5719 | GGCUGUG | CUGAUGAG | X | CGAA | AUGCCAU | AUGGCAU | U | CACAGCC |
| 5720 | AGGCUGU | CUGAUGAG | X | CGAA | AAUGCCA | UGGCAUU | C | ACAGCCU |
| 5728 | GGUGAUA | CUGAUGAG | X | CGAA | AGGCUGU | ACAGCCU | C | UAUCACC |
| 5730 | CUGGUGA | CUGAUGAG | X | CGAA | AGAGGCU | AGCCUCU | A | UCACCAG |
| 5732 | GACUGGU | CUGAUGAG | X | CGAA | AUAGAGG | CCUCUAU | C | ACCAGUC |
| 5739 | GUGAGCG | CUGAUGAG | X | CGAA | ACUGGUG | CACCAGU | C | CGCUCAC |
| 5744 | GGGUGGU | CUGAUGAG | X | CGAA | AGCGGAC | GUCCGCU | C | ACCACCC |
| 5757 | AGGAGGG | CUGAUGAG | X | CGAA | AUUCUGG | CCAGAAU | A | CCCUCCU |
| 5762 | UGAACAG | CUGAUGAG | X | CGAA | AGGGUAU | AUACCCU | C | CUGUUCA |
| 5774 | CCCCUAA | CUGAUGAG | X | CGAA | AUGUUGA | UCAACAU | C | UUAGGGG |
| 5776 | UCCCCCU | CUGAUGAG | X | CGAA | AGAUGUU | AACAUCU | U | AGGGGGA |
| 5777 | AUCCCCC | CUGAUGAG | X | CGAA | AAGAUGU | ACAUCUU | A | GGGGGAU |
| 5796 | GCGAGUU | CUGAUGAG | X | CGAA | AGCAGCC | GGCUGCU | C | AACUCGC |
| 5808 | GCACUGG | CUGAUGAG | X | CGAA | AGGAGCG | CGCUCCU | C | CCAGUGC |
| 5820 | AAGGCCG | CUGAUGAG | X | CGAA | AGCAGCA | UGCUGCU | U | CGGCCUU |
| 5885 | UGUCCAC | CUGAUGAG | X | CGAA | AGCACCU | AGGUGCU | U | GUGGACA |
| 5894 | CCGCCAG | CUGAUGAG | X | CGAA | AUGUCCA | UGGACAU | U | CUGGCGG |
| 5895 | CCCGCCA | CUGAUGAG | X | CGAA | AAUGUCC | GGACAUU | C | UGGCGGG |
| 5986 | AGGGAGC | CUGAUGAG | X | CGAA | AGUUAAC | GUUAACU | U | GCUCCCU |
| 5999 | GGGAGAG | CUGAUGAG | X | CGAA | AUGGCAG | CUGCCAU | C | CUCUCCC |
| 6002 | CGGGGGA | CUGAUGAG | X | CGAA | AGGAUGG | CCAUCCU | C | UCCCCCG |
| 6101 | CGAACGC | CUGAUGAG | X | CGAA | AUCAGCC | GGCUGAU | A | GCGUUCG |
| 6112 | ACCCCGC | CUGAUGAG | X | CGAA | AAGCGAA | UUCGCUU | C | GCGGGGU |
| 6120 | ACGUGGU | CUGAUGAG | X | CGAA | ACCCCGC | GCGGGGU | A | ACCACGU |
| 6128 | UGGGGGA | CUGAUGAG | X | CGAA | ACGUGGU | ACCACGU | U | UCCCCCA |
| 6129 | GUGGGGG | CUGAUGAG | X | CGAA | AACGUGG | CCACGUU | U | CCCCCAC |
| 6130 | CGUGGGG | CUGAUGAG | X | CGAA | AAACGUG | CACGUUU | C | CCCCACG |
| 6142 | AGGCACG | CUGAUGAG | X | CGAA | AGUGCGU | ACGCACU | A | CGUGCCU |
| 6173 | UCUGAGU | CUGAUGAG | X | CGAA | ACACGUG | CACGUGU | A | ACUCAGA |
| 6177 | AGGAUCU | CUGAUGAG | X | CGAA | AGUUACA | UGUAACU | C | AGAUCCU |
| 6182 | UGGAGAG | CUGAUGAG | X | CGAA | AUCUGAG | CUCAGAU | C | CUCUCCA |
| 6185 | GGCUGGA | CUGAUGAG | X | CGAA | AGGAUCU | AGAUCCU | C | UCCAGCC |
| 6187 | GAGGCUG | CUGAUGAG | X | CGAA | AGAGGAU | AUCCUCU | C | CAGCCUC |
| 6194 | UGAUGGU | CUGAUGAG | X | CGAA | AGGCUGG | CCAGCCU | C | ACCAUCA |
| 6200 | GCUGAGU | CUGAUGAG | X | CGAA | AUGGUGA | UCACCAU | C | ACUCAGC |
| 6204 | AGCAGCU | CUGAUGAG | X | CGAA | AGUGAUG | CAUCACU | C | AGCUGCU |
| 6221 | ACUGGUG | CUGAUGAG | X | CGAA | AGCCUCU | AGAGGCU | U | CACCAGU |
| 6222 | CACUGGU | CUGAUGAG | X | CGAA | AAGCCUC | GAGGCUU | C | ACCAGUG |
| 6233 | CCUCAUU | CUGAUGAG | X | CGAA | AUCCACU | AGUGGAU | U | AAUGAGG |
| 6234 | UCCUCAU | CUGAUGAG | X | CGAA | AAUCCAC | GUGGAUU | A | AUGAGGA |
| 6247 | UGGCGUG | CUGAUGAG | X | CGAA | AGCAGUC | GACUGCU | C | CACGCCA |
| 6259 | CGAGCCG | CUGAUGAG | X | CGAA | AGCAUGG | CCAUGCU | C | CGGCUCG |
| 6265 | UAGCCAC | CUGAUGAG | X | CGAA | AGCCGGA | UCCGGCU | C | GUGGCUA |
| 6272 | CAUCCUU | CUGAUGAG | X | CGAA | AGCCACG | CGUGGCU | A | AAGGAUG |
| 6281 | AGUCCCA | CUGAUGAG | X | CGAA | ACAUCCU | AGGAUGU | U | UGGGACU |
| 6282 | CAGUCCC | CUGAUGAG | X | CGAA | AACAUCC | GGAUGUU | U | GGGACUG |
| 6293 | CCGUGCA | CUGAUGAG | X | CGAA | AUCCAGU | ACUGGAU | A | UGCACGG |
| 6304 | GUCAGUC | CUGAUGAG | X | CGAA | ACACCGU | ACGGUGU | U | GACUGAC |
| 6313 | GGUCUUG | CUGAUGAG | X | CGAA | AGUCAGU | ACUGACU | U | CAAGACC |
| 6314 | AGGUCUU | CUGAUGAG | X | CGAA | AAGUCAG | CUGACUU | C | AAGACCU |
| 6326 | UGGACUG | CUGAUGAG | X | CGAA | AGCCAGG | CCUGGCU | C | CAGUCCA |
| 6331 | GAGCUUG | CUGAUGAG | X | CGAA | ACUGGAG | CUCCAGU | C | CAAGCUC |
| 6338 | UCGGCAG | CUGAUGAG | X | CGAA | AGCUUGG | CCAAGCU | C | CUGCCGA |
| 6349 | UCCCGGC | CUGAUGAG | X | CGAA | AUUUCGG | CCGAAAU | U | GCCGGGA |
| 6359 | AGAAAGG | CUGAUGAG | X | CGAA | ACUCCCG | CGGGAGU | C | CCUUUCU |
| 6363 | GAGAAGA | CUGAUGAG | X | CGAA | AGGGACU | AGUCCCU | U | UCUUCUC |
| 6364 | UGAGAAG | CUGAUGAG | X | CGAA | AAGGGAC | GUCCCUU | U | CUUCUCA |
| 6365 | AUGAGAA | CUGAUGAG | X | CGAA | AAAGGGA | UCCCUUU | C | UUCUCAU |
| 6367 | GCAUGAG | CUGAUGAG | X | CGAA | AGAAAGG | CCUUUCU | U | CUCAUGC |
| 6368 | GGCAUGA | CUGAUGAG | X | CGAA | AAGAAAG | CUUUCUU | C | UCAUGCC |
| 6370 | UUGGCAU | CUGAUGAG | X | CGAA | AGAAGAA | UUCUUCU | C | AUGCCAA |
| 6385 | UCCCUUG | CUGAUGAG | X | CGAA | ACCCGCG | CGCGGGU | A | CAAGGGA |
| 6395 | CCCGCCA | CUGAUGAG | X | CGAA | ACUCCCU | AGGGAGU | C | UGGCGGG |
| 6446 | GUCCGGU | CUGAUGAG | X | CGAA | AUUUGUG | CACAAAU | U | ACCGGAC |
| 6447 | UGUCCGG | CUGAUGAG | X | CGAA | AAUUUGU | ACAAAUU | A | CCGGACA |
| 6458 | CGUUUUU | CUGAUGAG | X | CGAA | ACAUGUC | GACAUGU | C | AAAAACG |
| 6468 | CUCAUGG | CUGAUGAG | X | CGAA | ACCGUUU | AAACGGU | U | CCAUGAG |
| 6469 | CCUCAUG | CUGAUGAG | X | CGAA | AACCGUU | AACGGUU | C | CAUGAUG |
| 6479 | GCCCAAC | CUGAUGAG | X | CGAA | AUCCUCA | UGAGGAU | C | GUUGGGC |
| 6482 | UAGGCCC | CUGAUGAG | X | CGAA | ACGAUCC | GGAUCGU | U | GGGCCUA |
| 6489 | CAGGUUU | CUGAUGAG | X | CGAA | AGGCCCA | UGGGCCU | A | AAACCUG |
| 6520 | GAUGGGG | CUGAUGAG | X | CGAA | ACGUUCC | GGAACGU | U | CCCCAUC |
| 6521 | UGAUGGG | CUGAUGAG | X | CGAA | AACGUUC | GAACGUU | C | CCCAUCA |
| 6527 | ACGCGUU | CUGAUGAG | X | CGAA | AUGGGGA | UCCCCAU | C | AACGCGU |
| 6535 | UGUGGUG | CUGAUGAG | X | CGAA | ACGCGUU | AACGCGU | A | CACCACA |
| 6559 | CGCCGGG | CUGAUGAG | X | CGAA | AGGGUGU | ACACCCU | C | CCCGGCG |
| 6610 | CUCCACG | CUGAUGAG | X | CGAA | ACUCUUC | GAAGAGU | A | CGUGGAG |
| 6620 | CCCGCGU | CUGAUGAG | X | CGAA | AUCUCCA | UGGAGAU | U | ACGCGGG |
| 6621 | ACCCGCG | CUGAUGAG | X | CGAA | AAUCUCC | GGAGAUU | A | CGCGGGU |
| 6654 | GUGGUCA | CUGAUGAG | X | CGAA | ACCCGUC | GACGGGU | A | UGACCAC |
| 6689 | GGGCCGG | CUGAUGAG | X | CGAA | ACCUGGC | GCCAGGU | C | CCGGCCC |
| 6781 | GACCUGG | CUGAUGAG | X | CGAA | AUGUGAC | GUCACAU | U | CCAGGUC |
| 6854 | UGGAAGU | CUGAUGAG | X | CGAA | AGCACUG | CAGUGCU | C | ACUUCCA |
| 6858 | AGCAUGG | CUGAUGAG | X | CGAA | AGUGAGC | GCUCACU | U | CCAUGCU |
| 6859 | GAGCAUG | CUGAUGAG | X | CGAA | AAGUGAG | CUCACUU | C | CAUGCUC |
| 6866 | GGUCGGU | CUGAUGAG | X | CGAA | AGCAUGG | CCAUGCU | C | ACCGACC |
| 6877 | AAUGUGG | CUGAUGAG | X | CGAA | AGGGGUC | GACCCCU | C | CCACAUU |
| 6884 | CUGCUGU | CUGAUGAG | X | CGAA | AUGUGGG | CCCACAU | U | ACAGCAG |
| 6885 | UCUGCUG | CUGAUGAG | X | CGAA | AAUGUGG | CCACAUU | A | CAGCAGA |
| 6900 | CUACGUU | CUGAUGAG | X | CGAA | AGCCGUC | GACGGCU | A | AACGUAG |
| 6945 | CUAGCUG | CUGAUGAG | X | CGAA | AGAGCUG | CAGCUCU | U | CAGCUAG |
| 6946 | GCUAGCU | CUGAUGAG | X | CGAA | AAGAGCU | AGCUCUU | C | AGCUAGC |
| 6951 | AAUUGGC | CUGAUGAG | X | CGAA | AGCUGAA | UUCAGCU | A | GCCAAUU |
| 6969 | UUCAAGG | CUGAUGAG | X | CGAA | AGGCGCA | UGCGCCU | U | CCUUGAA |
| 6970 | CUUCAAG | CUGAUGAG | X | CGAA | AAGGCGC | GCGCCUU | C | CUUGAAG |
| 6973 | UGCCUUC | CUGAUGAG | X | CGAA | AGGAAGG | CCUUCCU | U | GAAGGCA |
| 6990 | UGGUGGG | CUGAUGAG | X | CGAA | AGUGCAU | AUGCACU | A | CCCACCA |
| 7003 | GUCCGGG | CUGAUGAG | X | CGAA | AGUCAUG | CAUGACU | C | CCCGGAC |
| 7019 | CCUCGAU | CUGAUGAG | X | CGAA | AGGUCAG | CUGACCU | C | AUCGAGG |
| 7022 | UGGCCUC | CUGAUGAG | X | CGAA | AUGAGGU | ACCUCAU | C | GAGGCCA |
| 7064 | CACGGGU | CUGAUGAG | X | CGAA | AUGUUUC | GAAACAU | C | ACCCGUG |
| 7078 | AUUCUCU | CUGAUGAG | X | CGAA | ACUCCAC | GUGGAGU | C | AGAGAAU |
| 7086 | ACCACCU | CUGAUGAG | X | CGAA | AUUCUCU | AGAGAAU | A | AGGUGGU |
| 7094 | CCAAAAU | CUGAUGAG | X | CGAA | ACCACCU | AGGUGGU | A | AUUUUGG |
| 7097 | AGUCCAA | CUGAUGAG | X | CGAA | AGUACCA | UGGUAAU | U | UUGGACU |
| 7098 | GAGUCCA | CUGAUGAG | X | CGAA | AAUUACC | GGUAAUU | U | UGGACUC |
| 7099 | AGAGUCC | CUGAUGAG | X | CGAA | AAAUUAC | GUAAUUU | U | GGACUCU |
| 7105 | GUCGAAA | CUGAUGAG | X | CGAA | AGUCCAA | UUGGACU | C | UUUCGAC |
| 7107 | CGGUCCA | CUGAUGAG | X | CGAA | AGAGUCC | GGACUCU | U | UCGACCC |
| 7108 | CGGGUCG | CUGAUGAG | X | CGAA | AAGAGUC | GACUCUU | U | CGACCCG |
| 7109 | GCGGGUC | CUGAUGAG | X | CGAA | AAAGAGU | ACUCUUU | C | GACCCGC |
| 7147 | UGCAACG | CUGAUGAG | X | CGAA | AUACUUC | GAAGUAU | C | CGUUGCA |
| 7151 | CUGCUGC | CUGAUGAG | X | CGAA | ACGGAUA | UAUCCGU | U | GCAGCAG |
| 7163 | UUCGCAG | CUGAUGAG | X | CGAA | AUCUCUG | CAGAGAU | C | CUGCGAA |
| 7174 | CUUCUUG | CUGAUGAG | X | CGAA | AUUUUCG | CGAAAAU | C | CAAGAAG |
| 7183 | GGGGGGG | CUGAUGAG | X | CGAA | ACUUCUU | AAGAAGU | U | CCCCCCC |
| 7184 | CGGGGGG | CUGAUGAG | X | CGAA | AACUUCU | AGAAGUU | C | CCCCCCG |
| 7227 | AACAGUG | CUGAUGAG | X | CGAA | AGGGUUG | CAACCCU | C | CACUGUU |
| 7240 | UUUCCAG | CUGAUGAG | X | CGAA | ACUCUAA | UUAGAGU | C | CUGGAAA |
| 7308 | GGUAUUG | CUGAUGAG | X | CGAA | AGGGCCC | GGGCCCU | C | CAAUACC |
| 7313 | GAGGCGG | CUGAUGAG | X | CGAA | AUUGGAG | CUCCAAU | A | CCGCCUC |
| 7320 | UUCCGUG | CUGAUGAG | X | CGAA | AGGCGGU | ACCGCCU | C | CACGGAA |
| 7340 | UCAGAAC | CUGAUGAG | X | CGAA | ACCGUCC | GGACGGU | U | GUUCUGA |
| 7343 | CUGUCAG | CUGAUGAG | X | CGAA | ACAACCG | CGGUUGU | U | CUGACAG |
| 7344 | UCUGUCA | CUGAUGAG | X | CGAA | AACAACC | GGUUGUU | C | UGACAGA |
| 7363 | GGCAGAA | CUGAUGAG | X | CGAA | ACACGGU | ACCGUGU | C | UUCUGCC |
| 7365 | AAGGCAG | CUGAUGAG | X | CGAA | AGACACG | CGUGUCU | U | CUGCCUU |
| 7366 | CAAGGCA | CUGAUGAG | X | CGAA | AAGACAC | GUGUCUU | C | UGCCUUG |
| 7372 | CUCCGCC | CUGAUGAG | X | CGAA | AGGCAGA | UCUGCCU | U | GGCGGAG |
| 7405 | CGAUCCG | CUGAUGAG | X | CGAA | AGCUGCC | GGCAGCU | C | CGGAUCG |
| 7446 | UGAUCGG | CUGAUGAG | X | CGAA | AGGGGCG | CGCCCCU | C | CCGAUCA |
| 7452 | GAGGUCU | CUGAUGAG | X | CGAA | AUCGGGA | UCCCGAU | C | AGACCUC |
| 7459 | GUCGUCA | CUGAUGAG | X | CGAA | AGGUCUG | CAGACCU | C | UGACGAC |
| 7480 | AACGUCA | CUGAUGAG | X | CGAA | AUUCUUU | AAAGAAU | C | UGACGUU |
| 7487 | ACGACUC | CUGAUGAG | X | CGAA | ACGUCAG | CUGACGU | U | GAGUCGU |
| 7492 | GGAGUAC | CUGAUGAG | X | CGAA | ACUCAAC | GUUGAGU | C | GUACUCC |
| 7495 | GGAGGAG | CUGAUGAG | X | CGAA | ACGACUC | GAGUCGU | A | CUCCUCC |
| 7609 | CCAUGUG | CUGAUGAG | X | CGAA | AGGACAU | AUGUCCU | A | CACAUGG |
| 7631 | AUGGCGU | CUGAUGAG | X | CGAA | AUCAGGG | CCCUGAU | C | ACGCCAU |
| 7675 | GUUGCUC | CUGAUGAG | X | CGAA | ACGCGUU | AACGCGU | U | GAGCAAC |
| 7684 | CAGCAGA | CUGAUGAG | X | CGAA | AGUUGCU | AGCAACU | C | UCUGCUG |
| 7686 | CGCAGCA | CUGAUGAG | X | CGAA | AGAGUUG | CAACUCU | C | UGCUGCG |
| 7695 | UUGUGGU | CUGAUGAG | X | CGAA | ACGCAGC | GCUGCGU | C | ACCACAA |
| 7709 | UGGCAUA | CUGAUGAG | X | CGAA | ACCAUGU | ACAUGGU | C | UAUGCCA |
| 7711 | UGUGGCA | CUGAUGAG | X | CGAA | AGACCAU | AUGGUCU | A | UGCCACA |
| 7754 | CAAAGGU | CUGAUGAG | X | CGAA | ACCUUCU | AGAAGGU | C | ACCUUUG |
| 7759 | UCUGUCA | CUGAUGAG | X | CGAA | AGGUGAC | GUCACCU | U | UGACAGA |
| 7760 | GUCUGUC | CUGAUGAG | X | CGAA | AAGGUGA | UCACCUU | U | GACAGAC |
| 7802 | UCUCCUU | CUGAUGAG | X | CGAA | AGCACGU | ACGUGCU | C | AAGGAGA |
| 7825 | AACUGUG | CUGAUGAG | X | CGAA | ACGCCUU | AAGGCGU | C | CACAGUU |
| 7822 | UAGCCUU | CUGAUGAG | X | CGAA | ACUGUGG | CCACAGU | U | AAGGCUA |
| 7833 | UUAGCCU | CUGAUGAG | X | CGAA | AACUGUG | CACAGUU | A | AGGCUAA |
| 7844 | CGGAUAG | CUGAUGAG | X | CGAA | AGUUUAG | CUAAACU | U | CUAUCCG |
| 7845 | ACGGAUA | CUGAUGAG | X | CGAA | AAGUUUA | UAAACUU | C | UAUCCGU |
| 7884 | UUGGCCG | CUGAUGAG | X | CGAA | AUGUGGG | CCCACAU | U | CGGCCAA |
| 7885 | UUUGGCC | CUGAUGAG | X | CGAA | AAUGUGG | CCACAUU | C | GGCCAAA |
| 7922 | GGUUCCG | CUGAUGAG | X | CGAA | ACGUCCU | AGGACGU | C | CGGAACC |
| 7931 | UGCUGGA | CUGAUGAG | X | CGAA | AGGUUCC | GGAACCU | A | UCCAGCA |
| 7933 | CUUGCUG | CUGAUGAG | X | CGAA | AUAGGUU | AACCUAU | C | CAGCAAG |
| 7946 | UGUGGUU | CUGAUGAG | X | CGAA | AUGGCCU | AGGCCAU | U | AACCACA |
| 7947 | AUGUGGU | CUGAUGAG | X | CGAA | AAUGGCC | GGCCAUU | A | ACCACAU |
| 8000 | UGGUGUC | CUGAUGAG | X | CGAA | AUUGGUG | CACCAAU | U | GACACCA |
| 8012 | UUGCCAU | CUGAUGAG | X | CGAA | AUGGUGG | CCACCAU | C | AUGGCAA |
| 8030 | CGCAGAA | CUGAUGAG | X | CGAA | ACUUCAC | GUGAAGU | U | UUCUGCG |
| 8031 | ACGCAGA | CUGAUGAG | X | CGAA | AACUUCA | UGAAGUU | U | UCUGCGU |
| 8032 | GACGCAG | CUGAUGAG | X | CGAA | AAACUUC | GAAGUUU | U | CUGCGUC |
| 8033 | GGACGCA | CUGAUGAG | X | CGAA | AAAACUU | AAGUUUU | C | UGCGUCC |
| 8039 | CCGGUUG | CUGAUGAG | X | CGAA | ACGCAGA | UCUGCGU | C | CAACCGG |
| 8070 | AUAAGGC | CUGAUGAG | X | CGAA | AGCUGGC | GCCAGCU | C | GCCUUAU |
| 8081 | CUGGGAA | CUGAUGAG | X | CGAA | ACGAUAA | UUAUCGU | A | UUCCCAG |
| 8083 | GUCUGGG | CUGAUGAG | X | CGAA | AUACGAU | AUCGUAU | U | CCCAGAC |
| 8084 | GGUCUGG | CUGAUGAG | X | CGAA | AAUACGA | UCGUAUU | C | CCAGACC |
| 8099 | AUACACG | CUGAUGAG | X | CGAA | ACUCCCA | UGGGAGU | U | CGUGUAU |
| 8100 | CAUACAC | CUGAUGAG | X | CGAA | AACUCCC | GGGAGUU | C | GUGUAUG |
| 8105 | UCUCGCA | CUGAUGAG | X | CGAA | ACACGAA | UUCGUGU | A | UGCGAGA |
| 8121 | UCGUAAA | CUGAUGAG | X | CGAA | AGCCAUU | AAUGGCU | C | UUUACGA |
| 8123 | CGUCGUA | CUGAUGAG | X | CGAA | AGAGCCA | UGGCUCU | U | UACGACG |
| 8124 | ACCUCCU | CUGAUGAG | X | CGAA | AAGAGCC | GGCUCUU | U | ACGACGU |
| 8125 | CACGUCG | CUGAUGAG | X | CGAA | AAAGAGC | GCUCUUU | A | CGACGUG |
| 8135 | GGGUGGA | CUGAUGAG | X | CGAA | ACCACGU | ACGUGGU | C | UCCACCC |
| 8137 | AAGGGUG | CUGAUGAG | X | CGAA | AGACCAC | GUGGUCU | C | CACCCUU |
| 8144 | CCUGAGG | CUGAUGAG | X | CGAA | AGGGUGG | CCACCCU | U | CCUCAGG |
| 8145 | GCCUGAG | CUGAUGAG | X | CGAA | AAGGGUG | CACCCUU | C | CUCAGGC |
| 8148 | ACGGCCU | CUGAUGAG | X | CGAA | AGGAAGG | CCUUCCU | C | AGGCCGU |
| 8164 | GUACGAG | CUGAUGAG | X | CGAA | AGCCCAU | AUGGGCU | C | CUCGUAC |
| 8167 | UCCGUAC | CUGAUGAG | X | CGAA | AGGAGCC | GGCUCCU | C | GUACGGA |
| 8177 | AGUACUG | CUGAUGAG | X | CGAA | AAUCCGU | ACGGAUU | C | CAGUACU |
| 8185 | CCCAGGA | CUGAUGAG | X | CGAA | AGUACUG | CAGUACU | C | UCCUGGG |
| 8241 | AAGCCCA | CUGAUGAG | X | CGAA | AGGGCUU | AAGCCCU | A | UGGGCUU |
| 8248 | AUACGAG | CUGAUGAG | X | CGAA | AGCCCAU | AUGGGCU | U | CUCGUAU |
| 8249 | CAUACGA | CUGAUGAG | X | CGAA | AAGCCCA | UGGGCUU | C | UCGUAUG |
| 8251 | GUCAUAC | CUGAUGAG | X | CGAA | AGAAGCC | GGCUUCU | C | GUAUGAC |
| 8254 | GGUGUCA | CUGAUGAG | X | CGAA | ACGAGAA | UUCUCGU | A | UGACACC |
| 8269 | UGAGUCA | CUGAUGAG | X | CGAA | AGCAGCG | CGCUGCU | U | UGACUCA |
| 8270 | UUGAGUC | CUGAUGAG | X | CGAA | AAGCAGC | GCUGCUU | U | GACUCAA |
| 8275 | GACUGUU | CUGAUGAG | X | CGAA | AGUCAAA | UUUGACU | C | AACAGUC |
| 8282 | UCUCAGU | CUGAUGAG | X | CGAA | ACUGUUG | CAACAGU | C | ACUGAGA |
| 8297 | CAACACG | CUGAUGAG | X | CGAA | AUGUCGC | GCGACAU | C | CGUGUUG |
| 8303 | ACUCCUC | CUGAUGAG | X | CGAA | ACACGGA | UCCGUGU | U | GAGGAGU |
| 8311 | GUAGAUU | CUGAUGAG | X | CGAA | ACUCCUC | GAGGAGU | C | AAUCUAC |
| 8315 | AUUGGUA | CUGAUGAG | X | CGAA | AUUGACU | AGUCAAU | C | UACCAAU |
| 8317 | ACAUUGG | CUGAUGAG | X | CGAA | AGAUUGA | UCAAUCU | A | CCAAUGU |
| 8325 | AAGUCAC | CUGAUGAG | X | CGAA | ACAUUGG | CCAAUGU | U | GUGACUU |
| 8332 | GGGGGCC | CUGAUGAG | X | CGAA | AGUCACA | UGUGACU | U | GGCCCCC |
| 8400 | UUUGAAU | CUGAUGAG | X | CGAA | AGUCAGG | CCUGACU | A | AUUCAAA |
| 8403 | CCUUUUG | CUGAUGAG | X | CGAA | AUUAGUC | GACUAAU | U | CAAAAGG |
| 8404 | CCCUUUU | CUGAUGAG | X | CGAA | AAUUAGU | ACUAAUU | C | AAAAGGG |
| 8472 | GUGAGGG | CUGAUGAG | X | CGAA | AUUGCCG | CGGCAAU | A | CCCUCAC |
| 8477 | AGCAUGU | CUGAUGAG | X | CGAA | AGGGUAU | AUACCCU | C | ACAUGCU |
| 8485 | UUUCAAG | CUGAUGAG | X | CGAA | AGCAUGU | ACAUGCU | A | CUUGAAA |
| 8488 | GGCUUUC | CUGAUGAG | X | CGAA | AGUAGCA | UGCUACU | U | GAAAGCC |
| 8565 | UCACAGA | CUGAUGAG | X | CGAA | AACGACA | UGUCGUU | A | UCUGUGA |
| 8567 | UUUCACA | CUGAUGAG | X | CGAA | AUAACGA | UCGUUAU | C | UGUGAAA |
| 8606 | AGACUCG | CUGAUGAG | X | CGAA | AGGCUCG | CGAGCCU | A | CGAGUCU |
| 8612 | CCGUGAA | CUGAUGAG | X | CGAA | ACUCGUA | UACGAGU | C | UUCACGG |
| 8614 | CUCCGUG | CUGAUGAG | X | CGAA | AGACUCG | CGAGUCU | U | CACGGAG |
| 8615 | CCUCCGU | CUGAUGAG | X | CGAA | AAGACUC | GAGUCUU | C | ACGGAGG |
| 8625 | CUAGUCA | CUGAUGAG | X | CGAA | AGCCUCC | GGAGGCU | A | UGACUAG |
| 8631 | GAGUACC | CUGAUGAG | X | CGAA | AGUCAUA | UAUGACU | A | GGUACUC |
| 8635 | GGCAGAG | CUGAUGAG | X | CGAA | ACCUAGU | ACUAGGU | A | CUCUGCC |
| 8677 | CAACUCC | CUGAUGAG | X | CGAA | AGUCGUA | UACGACU | U | GGAGUUG |
| 8683 | UGUUAUC | CUGAUGAG | X | CGAA | ACUCCAA | UUGGAGU | U | GAUAACA |
| 8687 | AUGAUGU | CUGAUGAG | X | CGAA | AUCAACU | AGUUGAU | A | ACAUCAU |
| 8692 | GGAGCAU | CUGAUGAG | X | CGAA | AUGUUAU | AUAACAU | C | AUGCUCC |
| 8710 | CGCGACC | CUGAUGAG | X | CGAA | ACACGUU | AACGUGU | C | GGUCGCG |
| 8714 | CGUGCGC | CUGAUGAG | X | CGAA | ACCGACA | UGUCGGU | C | GCGCACG |
| 8743 | GAGGUAG | CUGAUGAG | X | CGAA | ACACUCU | AGAGUGU | A | CUACCUC |
| 8746 | AGUGAGG | CUGAUGAG | X | CGAA | AGUACAC | GUGUACU | A | CCUCACU |
| 8750 | CACGAGU | CUGAUGAG | X | CGAA | AGGUAGU | ACUACCU | C | ACUCGUG |
| 8754 | GGAUCAC | CUGAUGAG | X | CGAA | AGUGAGG | CCUCACU | C | GUGAUCC |
| 8760 | GUGGUGG | CUGAUGAG | X | CGAA | AUCACGA | UCGUGAU | C | CCACCAC |
| 8799 | GUGUGUC | CUGAUGAG | X | CGAA | AGCUGUC | GACAGCU | A | GACACAC |
| 8808 | UUGACUG | CUGAUGAG | X | CGAA | AGUGUGU | ACACACU | C | CAGUCAA |
| 8813 | AGGAGUU | CUGAUGAG | X | CGAA | ACUGGAG | CUCCAGU | C | AACUCCU |
| 8818 | UAGCCAG | CUGAUGAG | X | CGAA | AGUUGAC | GUCAACU | C | CUGGCUA |
| 8825 | UGUUGCC | CUGAUGAG | X | CGAA | AGCCAGG | CCUGGCU | A | GGCAACA |
| 8834 | ACAUGAU | CUGAUGAG | X | CGAA | AUGUUGC | GCAACAU | C | AUCAUGU |
| 8837 | CAUACAU | CUGAUGAG | X | CGAA | AUGAUGU | ACAUCAU | C | AUGUAUG |
| 8870 | UCAUCAA | CUGAUGAG | X | CGAA | AUCAUCC | GGAUGAU | U | UUGAUGA |
| 8872 | AGUCAUC | CUGAUGAG | X | CGAA | AAAUCAU | AUGAUUU | U | GAUGACU |
| 8884 | GGAGAAG | CUGAUGAG | X | CGAA | AGUGAGU | ACUCACU | U | CUUCUCC |
| 8885 | UGGAGAA | CUGAUGAG | X | CGAA | AAGUGAG | CUCACUU | C | UUCUCCA |
| 8887 | GAUGGAG | CUGAUGAG | X | CGAA | AGAAGUG | CACUUCU | U | CUCCAUC |
| 8888 | GGAUGGA | CUGAUGAG | X | CGAA | AAGAAGU | ACUUCUU | C | UCCAUCC |
| 8890 | AAGGAUG | CUGAUGAG | X | CGAA | AGAAGAA | UUCUUCU | C | CAUCCUG |
| 8894 | CUAGAAG | CUGAUGAG | X | CGAA | AUGGAGA | UCUCCAU | C | CUUCUAG |
| 8897 | GGGCUAG | CUGAUGAG | X | CGAA | AGGAUGG | CCAUCCU | U | CUAGCCC |
| 8898 | UGGGCUA | CUGAUGAG | X | CGAA | AAGGAUG | CAUCCUU | C | UAGCCCA |
| 8900 | CCUGGGC | CUGAUGAG | X | CGAA | AGAAGGA | UCCUUCU | A | GCCCAGG |
| 8915 | CCUUUUC | CUGAUGAG | X | CGAA | AGCUGUU | AACAGCU | U | GAAAAGG |
| 8952 | AUGGAGU | CUGAUGAG | X | CGAA | ACAGGCC | GGCCUGU | U | ACUCCAU |
| 8953 | AAUGGAG | CUGAUGAG | X | CGAA | AACAGGC | GCCUGUU | A | CUCCAUU |
| 8956 | CUCAAUG | CUGAUGAG | X | CGAA | AGUAACA | UGUUACU | C | CAUUGAG |
| 8960 | GUGGCUC | CUGAUGAG | X | CGAA | AUGGAGU | ACUCCAU | U | GAGCCAC |
| 8969 | GUAGGUC | CUGAUGAG | X | CGAA | AGUGGCU | AGCCACU | U | GACCUAC |
| 8975 | UCUGAGG | CUGAUGAG | X | CGAA | AGGUCAA | UUGACCU | A | CCUCAGA |
| 8979 | AUGAUCU | CUGAUGAG | X | CGAA | AGGUAGG | CCUACCU | C | AGAUCAU |
| 8984 | GUUGAAU | CUGAUGAG | X | CGAA | AUCUGAG | CUCAGAU | C | AUUCAAC |
| 8987 | GUCGUUG | CUGAUGAG | X | CGAA | AUGAUCU | AGAUCAU | U | CAACGAC |
| 8988 | AGUCGUU | CUGAUGAG | X | CGAA | AAUGAUC | GAUCAUU | C | AACGACU |
| 8996 | GACCAUG | CUGAUGAG | X | CGAA | AGUCGUU | AACGACU | C | CAUGGUC |
| 9003 | GCGCUAA | CUGAUGAG | X | CGAA | ACCAUGG | CCAUGGU | C | UUAGCGC |
| 9005 | AUGCGCU | CUGAUGAG | X | CGAA | AGACCAU | AUGGUCU | U | AGCGCAU |
| 9006 | AAUGCGC | CUGAUGAG | X | CGAA | AAGACCA | UGGUCUU | A | GCGCAUU |
| 9013 | GAGUGAG | CUGAUGAG | X | CGAA | AUGCGCU | AGCGCAU | U | CUCACUC |
| 9014 | GGAGUGA | CUGAUGAG | X | CGAA | AAUGCGC | GCGCAUU | C | UCACUCC |
| 9016 | AUGGAGU | CUGAUGAG | X | CGAA | AGAAUGC | GCAUUCU | C | ACUCCAU |
| 9020 | AACUAUG | CUGAUGAG | X | CGAA | AGUGAGA | UCUCACU | C | CAUAGUU |
| 9024 | GAGUAAC | CUGAUGAG | X | CGAA | AUGGAGU | ACUCCAU | A | GUUACUC |
| 9027 | GGAGAGU | CUGAUGAG | X | CGAA | ACUAUGG | CCAUAGU | U | ACUCUCC |
| 9028 | UGGAGAG | CUGAUGAG | X | CGAA | AACUAUG | CAUAGUU | A | CUCUCCA |
| 9032 | ACCUGGA | CUGAUGAG | X | CGAA | AGUAACU | AGUUACU | C | UCCAGGU |
| 9033 | UCACCUG | CUGAUGAG | X | CGAA | AGAGUAA | UUACUCU | C | CAGGUGA |
| 9044 | CCCUAUU | CUGAUGAG | X | CGAA | AUCUCAC | GUGAGAU | C | AAUAGGG |
| 9048 | GCCACCC | CUGAUGAG | X | CGAA | ACUGAUC | GAUCAAU | A | GGGUGGC |
| 9057 | AGGCAUG | CUGAUGAG | X | CGAA | AGCCACC | GGUGGCU | U | CAUGCCU |
| 9058 | GAGGCAU | CUGAUGAG | X | CGAA | AAGCCAC | GUGGCUU | C | AUGCCUC |
| 9105 | CUGGCCC | CUGAUGAG | X | CGAA | AUGUCUC | GAGACAU | C | GGGCCAG |
| 9169 | GAAGAGG | CUGAUGAG | X | CGAA | ACUUGCC | GGCAAGU | A | CCUCUUC |
| 9173 | AGUUGAA | CUGAUGAG | X | CGAA | AGGUACU | AGUACCU | C | UUCAACU |
| 9175 | CCAGUUG | CUGAUGAG | X | CGAA | AGAGGUA | UACCUCU | U | CAACUGG |
| 9176 | CCCAGUU | CUGAUGAG | X | CGAA | AAGAGGU | ACCUCUU | C | AACUGGG |
| 9188 | UGGUCCU | CUGAUGAG | X | CGAA | ACUGCCC | GGGCAGU | A | AGGACCA |
| 9200 | UGAGUUU | CUGAUGAG | X | CGAA | AGCUUGG | CCAAGCU | C | AAACUCA |
| 9206 | UUGGAGU | CUGAUGAG | X | CGAA | AGUUUGA | UCAAACU | C | ACUCCAA |
| 9210 | GGGAUUG | CUGAUGAG | X | CGAA | AGUGAGU | ACUCACU | C | CAAUCCC |
| 9215 | CGGCCGG | CUGAUGAG | X | CGAA | AUUGGAG | CUCCAAU | C | CCGGCCG |
| 9261 | CCGCUGU | CUGAUGAG | X | CGAA | ACCAGCA | UGCUGGU | U | ACAGCGG |
| 9262 | CCCGCUG | CUGAUGAG | X | CGAA | AACCAGC | GCUGGUU | A | CAGCGGG |
| 9294 | CGGGCAC | CUGAUGAG | X | CGAA | AGACAGG | CCUGUCU | C | GUGCCCG |
| 9313 | CCACAUA | CUGAUGAG | X | CGAA | ACCAGCG | CGCUGGU | U | UAUGUGG |
| 9314 | ACCACAU | CUGAUGAG | X | CGAA | AACCAGC | GCUGGUU | U | AUGUGGU |
| 9315 | CACCACA | CUGAUGAG | X | CGAA | AAACCAG | CUGGUUU | A | UGUGGUG |
| 9409 | AAAAGGG | CUGAUGAG | X | CGAA | AUGGCCU | AGGCCAU | C | CCCUUUU |
| 9414 | AAAAAAA | CUGAUGAG | X | CGAA | AGGGGAU | AUCCCCU | U | UUUUUUU |

Where “X” repesents stem II region of a HH ribozyme (Hertel et al., 1992 Nucleic Acids Res. 20: 3252). The length of stem II may be 2 base-pairs.

TABLE VII
HCV Hairpin (HP) Ribozyme and Target Sequence

| Pos. | Ribozyme Sequence | Substrate |
|---|---|---|
| 10 | CCCCCA AGAA GGGG ACCAGAGAAACA X GUACAUUACCUGGUA | CCCC CGAU UGGGGG |
| 59 | CGUGAA AGAA GUAG ACCAGAGAAACA X GUACAUUACCUGGUA | CUAC UGUC UUCACG |
| 109 | CCUGGA AGAA GCAC ACCAGAGAAACA X GUACAUUACCUGGUA | GUGC AGCC UCCAGG |
| 209 | GCAUUG AGAA GGUU ACCAGAGAAACA X GUACAUUACCUGGUA | AACC CGCU CAAUGC |
| 290 | CUAUCA AGAA GUAC ACCAGAGAAACA X GUACAUUACCUGGUA | GUAC UGCC UGAUAG |
| 390 | GUGGGC AGAA GUAG ACCAGAGAAACA X GUACAUUACCUGGUA | CUAC CGCC GCCCAC |
| 393 | CCUGUG AGAA GCGG ACCAGAGAAACA X GUACAUUACCUGGUA | CCGC CGCC CACAGG |
| 427 | CCAACG AGAA GACC ACCAGAGAAACA X GUACAUUACCUGGUA | GGUC AGAU CGUUGG |
| 505 | GGUUGC AGAA GUUC ACCAGAGAAACA X GUACAUUACCUGGUA | GAAC GGUC GCAACC |
| 549 | CCUCGG AGAA GCGA ACCAGAGAAACA X GUACAUUACCUGGUA | UCGC CGAC CCGAGG |
| 574 | UACCCA AGAA GAGC ACCAGAGAAACA X GUACAUUACCUGGUA | GCUC AGCC UGGGUA |
| 645 | GCCGGG AGAA GCGG ACCAGAGAAACA X GUACAUUACCUGGUA | CCGC GGCU CCCGGC |
| 652 | CAACUA AGAA GGGA ACCAGAGAAACA X GUACAUUACCUGGUA | UCCC GGCC UAGUUG |
| 671 | CCGGGG AGAA GUGG ACCAGAGAAACA X GUACAUUACCUGGUA | CCAC GGAC CCCCGG |
| 726 | CGGCGA AGAA GCAC ACCAGAGAAACA X GUACAUUACCUGGUA | GUGC GGCU UCGCCG |
| 734 | CAUGAG AGAA GCGA ACCAGAGAAACA X GUACAUUACCUGGUA | UCGC CGAC CUCAUG |
| 754 | CCGACG AGAA GAAU ACCAGAGAAACA X GUACAUUACCUGGUA | AUUC CGCU CGUCGG |
| 852 | AAGAGC AGAA GGGC ACCAGAGAAACA X GUACAUUACCUGGUA | GCCC GGUU GCUCUU |
| 883 | CAGGAC AGAA GGGC ACCAGAGAAACA X GUACAUUACCUGGUA | GCCC UGCU GUCCUG |
| 886 | AAACAG AGAA GCAG ACCAGAGAAACA X GUACAUUACCUGGUA | CUGC UGUC CUGUUU |
| 891 | UGGUCA AGAA GGAC ACCAGAGAAACA X GUACAUUACCUGGUA | GUCC UGUU UGACCA |
| 905 | AGCGGA AGAA GGGA ACCAGAGAAACA X GUACAUUACCUGGUA | UCCC AGCU UCCGCU |
| 911 | CUGAUA AGAA GAAG ACCAGAGAAACA X GUACAUUACCUGGUA | CUUC CGCU UAUCAG |
| 960 | AGUUGG AGAA GUCA ACCAGAGAAACA X GUACAUUACCUGGUA | UGAC UGCU CCAACU |
| 1050 | CCCAAC AGAA GGAG ACCAGAGAAACA X GUACAUUACCUGGUA | CUCC CGUU GUUGGG |
| 1145 | GAAAGC AGAA GCCC ACCAGAGAAACA X GUACAUUACCUGGUA | GGGC GGCC GCUUUC |
| 1148 | ACAGAA AGAA GCCG ACCAGAGAAACA X GUACAUUACCUGGUA | CGGC CGCU UUCUGU |
| 1155 | UGGCGG AGAA GAAA ACCAGAGAAACA X GUACAUUACCUGGUA | UUUC UGUU CCGCCA |
| 1185 | AAACGG AGAA GCAG ACCAGAGAAACA X GUACAUUACCUGGUA | CUGC GGAU CCGUUU |
| 1190 | GAGGAA AGAA GAUC ACCAGAGAAACA X GUACAUUACCUGGUA | GAUC CGUU UUCCUC |
| 1207 | GUGAAC AGAA GGGA ACCAGAGAAACA X GUACAUUACCUGGUA | UCCC AGUU GUUCAC |
| 1331 | CACUAG AGAA GUUG ACCAGAGAAACA X GUACAUUACCUGGUA | CAAC AGCC CUAGUG |
| 1357 | UGUGGG AGAA GGAG ACCAGAGAAACA X GUACAUUACCUGGUA | CUCC GGAU CCCACA |
| 1370 | AUCCAC AGAA GCUU ACCAGAGAAACA X GUACAUUACCUGGUA | AAGC UGUC GUGGAU |
| 1562 | UCUCUG AGAA GGCC ACCAGAGAAACA X GUACAUUACCUGGUA | GGCC GGCC CAGAGA |
| 1576 | UUUAUG AGAA GGAU ACCAGAGAAACA X GUACAUUACCUGGUA | AUCC AGCU CAUAAA |
| 1596 | UGUGCC AGAA GCCA ACCAGAGAAACA X GUACAUUACCUGGUA | UGGC AGCU GGCACA |
| 1616 | GUUCAG AGAA GUCC ACCAGAGAAACA X GUACAUUACCUGGUA | GGAC UGCC CUGAAC |
| 1663 | GCGUAG AGAA GUGC ACCAGAGAAACA X GUACAUUACCUGGUA | GCAC UGUU CUACGC |
| 1692 | CUGGGC AGAA GGAC ACCAGAGAAACA X GUACAUUACCUGGUA | GUCC GGAU GCCCAG |
| 1713 | AGCUGC AGAA GGCC ACCAGAGAAACA X GUACAUUACCUGGUA | GGCC AGCU GCAGCU |
| 1719 | CGAUGG AGAA GCAG ACCAGAGAAACA X GUACAUUACCUGGUA | CUGC AGCU CCAUCG |
| 1797 | AAUGCC AGAA GUAA ACCAGAGAAACA X GUACAUUACCUGGUA | UUAC UGCU GGCAUU |
| 1863 | GGGUGA AGAA GUAC ACCAGAGAAACA X GUACAUUACCUGGUA | GUAC UGUU UCACCC |
| 1880 | CACUAC AGAA GGGC ACCAGAGAAACA X GUACAUUACCUGGUA | GCCC UGUU GUAGUG |
| 1898 | GGACCG AGAA GUCG ACCAGAGAAACA X GUACAUUACCUGGUA | CGAC CGAU CGGUCC |
| 1903 | GCACCG AGAA GAUC ACCAGAGAAACA X GUACAUUACCUGGUA | GAUC GGUC CGGUGC |
| 1943 | CAGCAC AGAA GUCU ACCAGAGAAACA X GUACAUUACCUGGUA | AGAC AGAU GUGCUG |
| 1951 | UUGAGA AGAA GCAC ACCAGAGAAACA X GUACAUUACCUGGUA | GUGC UGCU UCUCAA |
| 1969 | UGUGGC AGAA GCGU ACCAGAGAAACA X GUACAUUACCUGGUA | ACGC GGCC GCCACA |
| 2082 | CCGUGG AGAA GGUC ACCAGAGAAACA X GUACAUUACCUGGUA | GACC UGCC CCACGG |
| 2090 | AAAGCA AGAA GUGG ACCAGAGAAACA X GUACAUUACCUGGUA | CCAC GGAU UGCUUU |
| 2316 | GCUCCG AGAA GUCC ACCAGAGAAACA X GUACAUUACCUGGUA | GGAC AGAU CGGAGC |
| 2328 | GCAGCG AGAA GAGC ACCAGAGAAACA X GUACAUUACCUGGUA | GCUC AGCC CGCUGC |
| 2332 | AGCAGC AGAA GGCU ACCAGAGAAACA X GUACAUUACCUGGUA | AGCC CGCU GCUGCU |
| 2335 | GACAGC AGAA GCGG ACCAGAGAAACA X GUACAUUACCUGGUA | CCGC UGCU GCUGUC |
| 2338 | GUGGAC AGAA GCAG ACCAGAGAAACA X GUACAUUACCUGGUA | CUGC UGCU GUCCAC |
| 2341 | GUCGUG AGAA GCAG ACCAGAGAAACA X GUACAUUACCUGGUA | CUGC UGUC CACGAC |
| 2370 | UGAAGG AGAA GGGA ACCAGAGAAACA X GUACAUUACCUGGUA | UCCC UGUU CCUUCA |
| 2390 | GGACAG AGAA GGUA ACCAGAGAAACA X GUACAUUACCUGGUA | UACC GGCU CUGUCC |
| 2395 | CCAGUG AGAA GAGC ACCAGAGAAACA X GUACAUUACCUGGUA | GCUC UGUC CACUGG |
| 2465 | GGAGAC AGAA GCUG ACCAGAGAAACA X GUACAUUACCUGGUA | CAGC GGUU GUCUCC |
| 2522 | GCGCGC AGAA GCCA ACCAGAGAAACA X GUACAUUACCUGGUA | UGGC GGAC GCGCGC |
| 2541 | UCCACA AGAA GGCA ACCAGAGAAACA X GUACAUUACCUGGUA | UGCC UGCU UGUGGA |
| 2557 | GCUAUC AGAA GCAU ACCAGAGAAACA X GUACAUUACCUGGUA | AUGC UGCU GAUAGC |
| 2579 | CUCUAG AGAA GCCU ACCAGAGAAACA X GUACAUUACCUGGUA | AGGC CGCC CUAGAG |
| 2627 | AAUGCC AGAA GCUC ACCAGAGAAACA X GUACAUUACCUGGUA | GAGC GGAU GGCAUU |
| 2663 | GUACCA AGAA GCAC ACCAGAGAAACA X GUACAUUACCUGGUA | GUGC CGCC UGGUAC |
| 2725 | AGGAGC AGAA GCCA ACCAGAGAAACA X GUACAUUACCUGGUA | UGGC CGCU GCUCCU |
| 2728 | AGCAGG AGAA GCGG ACCAGAGAAACA X GUACAUUACCUGGUA | CCGC UGCU CCUGCU |
| 2734 | AGCAGG AGAA GGAG ACCAGAGAAACA X GUACAUUACCUGGUA | CUCC UGCU CCUGCU |
| 2740 | AACGCC AGAA GGAG ACCAGAGAAACA X GUACAUUACCUGGUA | CUCC UGCU GGCGUU |
| 2978 | UGGGUG AGAA GCAC ACCAGAGAAACA X GUACAUUACCUGGUA | GUGC GGCC CACCCA |
| 3016 | AUGGCG AGAA GGAG ACCAGAGAAACA X GUACAUUACCUGGUA | CUCC UGCU CGCCAU |
| 3030 | UGAGCG AGAA GAGA ACCAGAGAAACA X GUACAUUACCUGGUA | UCUC GGUC CGCUCA |
| 3034 | ACCAUG AGAA GACC ACCAGAGAAACA X GUACAUUACCUGGUA | GGUC CGCU CAUGGU |
| 3260 | GAAGAC AGAA GGCU ACCAGAGAAACA X GUACAUUACCUGGUA | AGCC CGUC GUCUUC |
| 3340 | GAGACG AGAA GUCC ACCAGAGAAACA X GUACAUUACCUGGUA | GGAC UGCC CGUCUC |
| 3344 | GGCGGA AGAA GGCA ACCAGAGAAACA X GUACAUUACCUGGUA | UGCC CGUC UCCGCC |
| 3350 | CCUUCG AGAA GAGA ACCAGAGAAACA X GUACAUUACCUGGUA | UCUC CGCC CGAAGG |
| 3383 | GCUAUC AGAA GGUC ACCAGAGAAACA X GUACAUUACCUGGUA | GACC GGCC GAUAGC |
| 3431 | GGCGUA AGAA GUGA ACCAGAGAAACA X GUACAUUACCUGGUA | UCAC GGCC UACGCC |
| 3581 | GUGGAA AGAA GUCC ACCAGAGAAACA X GUACAUUACCUGGUA | GGAC CGUC UUCCAC |
| 3597 | UCUUUG AGAA GGCG ACCAGAGAAACA X GUACAUUACCUGGUA | CGCC GGCU CAAAGA |
| 3615 | CUUUUG AGAA GGCU ACCAGAGAAACA X GUACAUUACCUGGUA | AGCC GGCC CAAAAG |
| 3669 | CAUGCC AGAA GACG ACCAGAGAAACA X GUACAUUACCUGGUA | CGUC GGCU GGCAUG |
| 3725 | AUAGAG AGAA GAGC ACCAGAGAAACA X GUACAUUACCUGGUA | GCUC GGAC CUCUAU |
| 3752 | AAUGAC AGAA GCAU ACCAGAGAAACA X GUACAUUACCUGGUA | AUGC UGAC GUCAUU |
| 3771 | CACCGC AGAA GCGC ACCAGAGAAACA X GUACAUUACCUGGUA | GCGC CGAC GCGGUG |
| 3783 | UCCCCC AGAA GUCA ACCAGAGAAACA X GUACAUUACCUGGUA | UGAC GGUC GGGGGA |
| 3799 | CUGGOG AGAA GUAG ACCAGAGAAACA X GUACAUUACCUGGUA | CUAC UGUC CCCCAG |
| 3807 | AGACGG AGAA GGGG ACCAGAGAAACA X GUACAUUACCUGGUA | CCCC AGAC CCGUCU |
| 3812 | AUAGGA AGAA GGUC ACCAGAGAAACA X GUACAUUACCUGGUA | GACC CGUC UCCUAU |
| 3847 | GGGCAG AGAA GUGG ACCAGAGAAACA X GUACAUUACCUGGUA | CCAC UGCU CUGCCC |
| 3852 | CCGAAG AGAA GAGC ACCAGAGAAACA X GUACAUUACCUGGUA | GCUC UGCC CUUCGG |
| 3887 | GCACAC AGAA GCCC ACCAGAGAAACA X GUACAUUACCUGGUA | GGGC UGCU GUGUGC |
| 3932 | AGACUC AGAA GGUA ACCAGAGAAACA X GUACAUUACCUGGUA | UACC CGUU GAGUCU |
| 3958 | ACCGGG AGAA GCAU ACCAGAGAAACA X GUACAUUACCUGGUA | AUGC GGUC CCCGGU |
| 3965 | CGUGAA AGAA GGGG ACCAGAGAAACA X GUACAUUACCUGGUA | CCCC GGUC UUCACG |
| 3992 | CGGUAC AGAA GGGG ACCAGAGAAACA X GUACAUUACCUGGUA | CCCC GGCC GUACCG |
| 4064 | GUACGC AGAA GGCA ACCAGAGAAACA X GUACAUUACCUGGUA | UGCC GGCU GCGUAC |
| 4076 | CCCUUG AGAA GCGU ACCAGAGAAACA X GUACAUUACCUGGUA | ACGC AGCC CAAGGG |
| 4112 | GGCGGC AGAA GAUG ACCAGAGAAACA X GUACAUUACCUGGUA | CAUC UGUU GCCGCC |
| 4163 | GUUGGG AGAA GUAC ACCAGAGAAACA X GUACAUUACCUGGUA | GUAC CGAC CCCAAC |
| 4244 | UCCACC AGAA GCAA ACCAGAGAAACA X GUACAUUACCUGGUA | UUGC CGAC GGUGGA |
| 4304 | AGUCGA AGAA GUUG ACCAGAGAAACA X GUACAUUACCUGGUA | CAAC UGAC UCGACU |
| 4334 | GUCCAG AGAA GUGC ACCAGAGAAACA X GUACAUUACCUGGUA | GCAC AGUC CUGGAC |
| 4355 | CGCUCC AGAA GUCU ACCAGAGAAACA X GUACAUUACCUGGUA | AGAC GGCU GGAGCG |
| 4366 | ACGACG AGAA GCGC ACCAGAGAAACA X GUACAUUACCUGGUA | GCGC GGCU CGUCGU |
| 4441 | GUGUUG AGAA GAGC ACCAGAGAAACA X GUACAUUACCUGGUA | GCUC UGUC CAACAC |
| 4621 | CCGCUA AGAA GUAU ACCAGAGAAACA X GUACAUUACCUGGUA | AUAC CGAC UAGCGG |
| 4652 | UAGAGC AGAA GUUG ACCAGAGAAACA X GUACAUUACCUGGUA | CAAC AGAC GCUCUA |
| 4724 | GAAAUC AGAA GUCU ACCAGAGAAACA X GUACAUUACCUGGUA | AGAC AGUC GAUUUC |
| 4734 | GAUCCA AGAA GAAA ACCAGAGAAACA X GUACAUUACCUGGUA | UUUC AGCU UGGAUC |
| 4861 | CCCGAG AGAA GUUC ACCAGAGAAACA X GUACAUUACCUGGUA | GAAC GGCC CUCGGG |
| 4886 | ACACAG AGAA GAAG ACCAGAGAAACA X GUACAUUACCUGGUA | CUUC GGUC CUGUGU |
| 4937 | AGUCUC AGAA GGCG ACCAGAGAAACA X GUACAUUACCUGGUA | CGCC CGCU GAGACU |
| 4988 | CUGGCA AGAA GGCA ACCAGAGAAACA X GUACAUUACCUGGUA | UGCC CGUC UGCCAG |
| 5059 | GUUUGG AGAA GGAA ACCAGAGAAACA X GUACAUUACCUGGUA | UUCC UGUC CCAAAC |
| 5179 | GGUUUA AGAA GUAU ACCAGAGAAACA X GUACAUUACCUGGUA | AUAC GGCU UAAACC |
| 5212 | CUAUAC AGAA GGGG ACCAGAGAAACA X GUACAUUACCUGGUA | CCCC UGCU GUAUAG |
| 5231 | AUUUUG AGAA GCUC ACCAGAGAAACA X GUACAUUACCUGGUA | GAGC CGUU CAAAAU |
| 5291 | CAGGUC AGAA GACA ACCAGAGAAACA X GUACAUUACCUGGUA | UGUC GGCC GACCUG |
| 5294 | CUCCAG AGAA GCCG ACCAGAGAAACA X GUACAUUACCUGGUA | CGGC CGAC CUGGAG |
| 5345 | GGCCAG AGAA GCAA ACCAGAGAAACA X GUACAUUACCUGGUA | UUGC AGCU CUGGCC |
| 5417 | AACAAC AGAA GGCC ACCAGAGAAACA X GUACAUUACCUGGUA | GGCC GGCU GUUGUU |
| 5420 | GGGAAC AGAA GCCG ACCAGAGAAACA X GUACAUUACCUGGUA | CGGC UGUU GUUCCC |
| 5509 | UCGGCG AGAA GCAU ACCAGAGAAACA X GUACAUUACCUGGUA | AUGC AGCU CGCCGA |
| 5521 | UGCUUG AGAA GCUC ACCAGAGAAACA X GUACAUUACCUGGUA | GAGC AGUU CAAGCA |
| 5576 | GGGAGC AGAA GCCU ACCAGAGAAACA X GUACAUUACCUGGUA | AGGC CGCU GCUCCC |
| 5579 | CACGGG AGAA GCGG ACCAGAGAAACA X GUACAUUACCUGGUA | CCGC UGCU CCCGUG |
| 5683 | UUCCCA AGAA GAGU ACCAGAGAAACA X GUACAUUACCUGGUA | ACUC UGCC UGGGAA |
| 5710 | AAUGCC AGAA GUGA ACCAGAGAAACA X GUACAUUACCUGGUA | UCAC UGAU GGCAUU |
| 5723 | GAUAGA AGAA GUGA ACCAGAGAAACA X GUACAUUACCUGGUA | UCAC AGCC UCUAUC |
| 5736 | UGAGCG AGAA GGUG ACCAGAGAAACA X GUACAUUACCUGGUA | CACC AGUC CGCUCA |
| 5740 | GUGGUG AGAA GACU ACCAGAGAAACA X GUACAUUACCUGGUA | AGUC CGCU CACCAC |
| 5764 | AUGUUG AGAA GGAG ACCAGAGAAACA X GUACAUUACCUGGUA | CUCC UGUU CAACAU |
| 5792 | GAGUUG AGAA GCCA ACCAGAGAAACA X GUACAUUACCUGGUA | UGGC UGCU CAACUC |
| 5816 | GGCCGA AGAA GCAC ACCAGAGAAACA X GUACAUUACCUGGUA | GUGC UGCU UCGGCC |
| 5822 | CACGAA AGAA GAAG ACCAGAGAAACA X GUACAUUACCUGGUA | CUUC GGCC UUCGUG |
| 5966 | GUCCUC AGAA GAGG ACCAGAGAAACA X GUACAUUACCUGGUA | CCUC CGCC GAGGAC |
| 6094 | GCUAUC AGAA GGUU ACCAGAGAAACA X GUACAUUACCUGGUA | AACC GGCU GAUAGC |
| 6178 | GAGAGG AGAA GAGU ACCAGAGAAACA X GUACAUUACCUGGUA | ACUC AGAU CCUCUC |
| 6189 | UGGUGA AGAA GGAG ACCAGAGAAACA X GUACAUUACCUGGUA | CUCC AGCC UCACCA |
| 6205 | UUCAGC AGAA GAGU ACCAGAGAAACA X GUACAUUACCUGGUA | ACUC AGCU GCUGAA |
| 6208 | CUCUUC AGAA GCUG ACCAGAGAAACA X GUACAUUACCUGGUA | CAGC UGCU GAAGAG |
| 6243 | GCGUGG AGAA GUCC ACCAGAGAAACA X GUACAUUACCUGGUA | GGAC UGCU CCACGC |
| 6261 | GCCACG AGAA GGAG ACCAGAGAAACA X GUACAUUACCUGGUA | CUCC GGCU CGUGGC |
| 6308 | CUUGAA AGAA GUCA ACCAGAGAAACA X GUACAUUACCUGGUA | UGAC UGAC UUCAAG |
| 6328 | AGCUUG AGAA GGAG ACCAGAGAAACA X GUACAUUACCUGGUA | CUCC AGUC CAAGCU |
| 6340 | AAUUUC AGAA GGAG ACCAGAGAAACA X GUACAUUACCUGGUA | CUCC UGCC GAAAUU |
| 6426 | CACAUG AGAA GGUG ACCAGAGAAACA X GUACAUUACCUGGUA | CACC UGCC CAUGUG |
| 6465 | UCAUGG AGAA GUUU ACCAGAGAAACA X GUACAUUACCUGGUA | AAAC GGUU CCAUGA |
| 6599 | CUCUUC AGAA GCCA ACCAGAGAAACA X GUACAUUACCUGGUA | UGGC UGCU GAAGAG |
| 6692 | UUCGGG AGAA GGGA ACCAGAGAAACA X GUACAUUACCUGGUA | UCCC GGCC CCCGAA |
| 6727 | CUGUGC AGAA GCAC ACCAGAGAAACA X GUACAUUACCUGGUA | GUGC GGUU GCACAG |
| 6753 | GGAGAG AGAA GCAC ACCAGAGAAACA X GUACAUUACCUGGUA | GUGC AGAC CUCUCC |
| 6817 | CAUGGG AGAA GUGA ACCAGAGAAACA X GUACAUUACCUGGUA | UCAC AGCU CCCAUG |
| 6839 | UGCCAC AGAA GGUU ACCAGAGAAACA X GUACAUUACCUGGUA | AACC GGAU GUGGCA |
| 6869 | GGAGGG AGAA GUGA ACCAGAGAAACA X GUACAUUACCUGGUA | UCAC CGAC CCCUCC |
| 6939 | CUGAAG AGAA GGCC ACCAGAGAAACA X GUACAUUACCUGGUA | GGCC AGCU CUUCAG |
| 7007 | GUCAGC AGAA GGGG ACCAGAGAAACA X GUACAUUACCUGGUA | CCCC GGAC GCUGAC |
| 7013 | GAUGAG AGAA GCGU ACCAGAGAAACA X GUACAUUACCUGGUA | ACGC UGAC CUCAUC |
| 7114 | GCUCGA AGAA OGUC ACCAGAGAAACA X GUACAUUACCUGGUA | GACC CGCU UCGAGC |
| 7148 | UGCUGC AGAA GAUA ACCAGAGAAACA X GUACAUUACCUGGUA | UAUC CGUU GCAGCA |
| 7214 | GUUGUA AGAA GGGC ACCAGAGAAACA X GUACAUUACCUGGUA | GCCC GGAU UACAAC |
| 7253 | GACGUA AGAA GGAC ACCAGAGAAACA X GUACAUUACCUGGUA | GUCC GGAC UACGUC |
| 7291 | GUGGUA AGAA GCAA ACCAGAGAAACA X GUACAUUACCUGGUA | UUGC CGCC UACCAC |
| 7315 | CGUGGA AGAA GUAU ACCAGAGAAACA X GUACAUUACCUGGUA | AUAC CGCC UCCACG |
| 7337 | CAGAAC AGAA GUCC ACCAGAGAAACA X GUACAUUACCUGGUA | GGAC GGUU GUCCUG |
| 7367 | CGCCAA AGAA GAAG ACCAGAGAAACA X GUACAUUACCUGGUA | CUUC UGCC UUGGCG |
| 7401 | AUCCGG AGAA GCCG ACCAGAGAAACA X GUACAUUACCUGGUA | CGGC AGCU CCGGAU |
| 7407 | CCGACG AGAA GGAG ACCAGAGAAACA X GUACAUUACCUGGUA | CUCC GGAU CGUCGG |
| 7415 | GUCAAC AGAA GACG ACCAGAGAAACA X GUACAUUACCUGGUA | CGUC GGCC GUGGAC |
| 7418 | GCUGUC AGAA GCCG ACCAGAGAAACA X GUACAUUACCUGGUA | CGGC CGUU GACAGC |
| 7439 | GGGAGG AGAA GUCG ACCAGAGAAACA X GUACAUUACCUGGUA | CGAC CGCC CCUCCC |
| 7448 | GGUCUG AGAA GGAG ACCAGAGAAACA X GUACAUUACCUGGUA | CUCC CGAU CAGACC |
| 7453 | UCAGAG AGAA GAUC ACCAGAGAAACA X GUACAUUACCUGGUA | GAUC AGAC CUCUGA |
| 7460 | ACCGUC AGAA GAGG ACCAGAGAAACA X GUACAUUACCUGGUA | CCUC UGAC GACGGU |
| 7481 | CUCAAC AGAA GAUU ACCAGAGAAACA X GUACAUUACCUGGUA | AAUC UGAC GUUGAG |
| 7535 | GCUGAG AGAA GGGU ACCAGAGAAACA X GUACAUUACCUGGUA | ACCC UGAU CUCAGC |
| 7593 | UUGAGC AGAA GACG ACCAGAGAAACA X GUACAUUACCUGGUA | CGUC UGCU GCUCAA |
| 7596 | ACADUG AGAA GCAG ACCAGAGAAACA X GUACAUUACCUGGUA | CUGC UGCU CAAUGU |
| 7627 | GGCGUG AGAA GGGC ACCAGAGAAACA X GUACAUUACCUGGUA | GCCC UGAU CACGCC |
| 7660 | UUGAUG AGAA GCUU ACCAGAGAAACA X GUACAUUACCUGGUA | AAGC UGCC CAUCAA |
| 7687 | UGACGC AGAA GAGA ACCAGAGAAACA X GUACAUUACCUGGUA | UCUC UGCU GCGUCA |
| 7764 | CUUGCA AGAA GUCA ACCAGAGAAACA X GUACAUUACCUGGUA | UGAC AGAC UGCAAG |
| 7870 | GGGGGC AGAA GCUU ACCAGAGAAACA X GUACAUUACCUGGUA | AAGC UGAC GCCCCC |
| 7956 | ACACGG AGAA GAUG ACCAGAGAAACA X GUACAUUACCUGGUA | CAUC CGCU CCGUGU |
| 7975 | UCUUCC AGAA GGUC ACCAGAGAAACA X GUACAUUACCUGGUA | GACC UGCU GGAAGA |
| 8066 | AAGGCG AGAA GGCU ACCAGAGAAACA X GUACAUUACCUGGUA | AGCC AGCU CGCCUU |
| 8087 | UCCCAG AGAA GGGA ACCAGAGAAACA X GUACAUUACCUGGUA | UCCC AGAC CUGGGA |
| 8172 | ACUGGA AGAA GUAC ACCAGAGAAACA X GUACAUUACCUGGUA | GUAC GGAU UCCAGU |
| 8262 | CAAAGC AGAA GGUG ACCAGAGAAACA X GUACAUUACCUGGUA | CACC CGCU GCUUUG |
| 8265 | AGUCAA AGAA GCGG ACCAGAGAAACA X GUACAUUACCUGGUA | CCGC UGCU UUGACU |
| 8374 | AUGUAG AGAA GCUC ACCAGAGAAACA X GUACAUUACCUGGUA | GAGC GGCU CUACAU |
| 8395 | GAAUUA AGAA GGGG ACCAGAGAAACA X GUACAUUACCUGGUA | CCCC UGAC UAAUUC |
| 8452 | CUAGUC AGAA GCAC ACCAGAGAAACA X GUACAUUACCUGGUA | GUGC UGAC GACUAG |
| 8501 | UCGACA AGAA GCAG ACCAGAGAAACA X GUACAUUACCUGGUA | CUGC GGCC UGUCGA |
| 8505 | CAGCUC AGAA GGCC ACCAGAGAAACA X GUACAUUACCUGGUA | GGCC UGUC GAGCUG |
| 8639 | GGGGGG AGAA GAGU ACCAGAGAAACA X GUACAUUACCUGGUA | ACUC UGCC CCCCCC |
| 8656 | GGUUGG AGAA GGUC ACCAGAGAAACA X GUACAUUACCUGGUA | GACC CGCC CCAACC |
| 8711 | GUGCGC AGAA GACA ACCAGAGAAACA X GUACAUUACCUGGUA | UGUC GGUC GCGCAC |
| 8911 | UUUUCA AGAA GUUC ACCAGAGAAACA X GUACAUUACCUGGUA | GAAC AGCU UGAAAA |
| 8935 | CCGUAG AGAA GACA ACCAGAGAAACA X GUACAUUACCUGGUA | UGUC AGAU CUACGG |
| 8980 | UGAAUG AGAA GAGG ACCAGAGAAACA X GUACAUUACCUGGUA | CCUC AGAU CAUUCA |
| 9082 | CGCAAG AGAA GUAC ACCAGAGAAACA X GUACAUUACCUGGUA | GUAC CGCC CUUGCG |
| 9133 | CCUUGG AGAA GUAG ACCAGAGAAACA X GUACAUUACCUGGUA | CUAC UGUC CCAAGG |
| 9218 | GGACGC AGAA GGGA ACCAGAGAAACA X GUACAUUACCUGGUA | UCCC GGCC GCGUCC |
| 9229 | AAGUCC AGAA GGGA ACCAGAGAAACA X GUACAUUACCUGGUA | UCCC AGCU GGACUU |
| 9243 | CGAACC AGAA GGAC ACCAGAGAAACA X GUACAUUACCUGGUA | GUCC AGCU GGUUCG |
| 9285 | GAGACA AGAA GUGA ACCAGAGAAACA X GUACAUUACCUGGUA | UCAC AGCC UGUCUC |
| 9289 | GCACGA AGAA GGCU ACCAGAGAAACA X GUACAUUACCUGGUA | AGCC UGUC UCGUGC |
| 9300 | AGCGGG AGAA GGCA ACCAGAGAAACA X GUACAUUACCUGGUA | UGCC CGAC CCCGCU |
| 9306 | UAAACC AGAA GGGU ACCAGAGAAACA X GUACAUUACCUGGUA | ACCC CGCU GGUUUA |
| 9358 | UUGGGG AGAA GGUA ACCAGAGAAACA X GUACAUUACCUGGUA | UACC UGCU CCCCAA |

Where “X”represents stem IV region of a HP ribozyme (Berzal-Herranze et al., 1993, EMBO.J. 12,2567). The length of stem IV may be 2 base-pairs.

TABLE VIII
Additional HCV Conserved Hammerhead ribozyme and target sequence

| Nos. | Name* | Pos.^{†} | Ribozyme | Substrate |
|---|---|---|---|---|
| 1 | HCV.C-48 | 278 | UUGGUGU CUGAUGAG X CGAA ACGUUUG | CAAACGU A ACACCAA |
| 2 | HCV.C-60 | 290 | UGUGGGC CUGAUGAG X CGAA ACGGUUG | CAACCGU C GCCCACA |
| 3 | HCV.C-175 | 409 | AGGUUGU CUGAUGAG X CGAA ACCGCUC | GAGCGGU C ACAACCU |
| 4 | HCV.3-118 | 9418 | AAAAAAA CUGAUGAG X CGAA AAAAAAA | UUUUUUU U UUUUUUU |
| 5 | HCV.3-145 | 9445 | UAAGAUG CUGAUGAG X CGAA AGCCACC | GGUGGCU C CAUCUUA |
| 6 | HCV.3-149 | 9449 | GGGCUAA CUGAUGAG X CGAA AUGGAGC | GCUCCAU C UUAGCCC |
| 7 | HCV.3-151 | 9451 | UAGGGCU CUGAUGAG X CGAA AGAUGGA | UCCAUCU U AGCCCUA |
| 8 | HCV.3-152 | 9452 | CUAGGGC CUGAUGAG X CGAA AAGAUGG | CCAUCUU A GCCCUAG |
| 9 | HCV.3-158 | 9458 | CCGUGAC CUGAUGAG X CGAA AGGGCUA | UAGCCCU A GUCACGG |
| 10 | HCV.3-161 | 9461 | UAGCCGU CUGAUGAG X CGAA ACUAGGG | CCCUAGU C ACGGCUA |
| 11 | HCV.3-168 | 9468 | UCACAGC CUGAUGAG X CGAA AGCCGUG | CACGGCU A GCUGUGA |
| 12 | HCV.3-181 | 9481 | GCUCACG CUGAUGAG X CGAA ACCUUUC | GAAAGGU C CGUGAGC |

Where “X” represents stem II region of a HH ribozyme (Hertel et al., 1992 Nucleic Acids Res. 20:3252). The length of stem II may be 2 base-pairs.
Core Reference Sequence for Nos. 1-3 = HPCCOPR (Acc#L38318) 1-600 bp
*Nucleotide 231 (8 nucleotide upstream of the initiator ATG) has been designated as “1” for the purpose of numbering ribozyme sites in the core portein coding region.
3′-NCR Reference Sequence for Nos. 4-12 = D85516 (Acc#D85516) 9301-9535 bp
*Nucleotide 9301 has been designated as “1” for the purpose of numbering ribozyme sites in the 3′NCR.
⁺position number reflects the reference sequence from HPCCOPR.

Where “X”represents stem II region of a HH ribozyme (Hertel et al., 1992 Nucleic Acids Res. 20: 3252). The length of stem II may be 2 base-pairs.

Core reference Sequence for Nos. 1-3=HPCCOPR (Acc#L38318) 1-600 bp *-Nucleotide 231 (8 nucleotide upstream of the initiator ATG) has been designated as “1” for the purpose of numbering ribozyme sites in the core protein coding region.

3′-NCR Reference Sequence for Nos. 4-12=D85516 (Acc#D85516) 9301-9535 bp *-Nucleotide 9301 has been designated as “1”for the purpose of numbering ribozyme sites in the 3′NCR.

↑-position number reflects the reference sequence from HPCCOPR.

TABLE IX
Inhibition of HCV RNA in OST7 cells Using Multiple Ribozyme Motifs

| Motif | RPI Number | F_{luc}/R_{luc} | SEM | Sequence |
|---|---|---|---|---|
| RPI Motif I | Irrelevant Control | 0.22 | 0.03 | auccuUGAUₛGGCAUACACUAUGCGCGaugaucugcaB |
| RPI Motif I | 18738 | 0.13 | 0.03 | acacuuGAUₛggcauGcacuaugcgcgauacuaacgcB |
| RPI Motif I | 18739 | 0.15 | 0.01 | cacgauGAUₛggcauGcacuaugcgcgacucauacuaB |
| RPI Motif I | 18740 | 0.15 | 0.01 | ggcuguGAUₛggcauGcacuaugcgcgacgacacucaB |
| RPI Motif I | 18746 | 0.10 | 0.02 | cccaauGAUₛggcauGcacuaugcgcgacuacucggcB |
| RPI Motif I | 18747 | 0.16 | 0.02 | uuucguGAUₛggcauGcacuaugcgcggacccaacacB |
| RPI Motif I | 18750 | 0.15 | 0.03 | ucagguGAUₛggcauGcacuaugcgcgaguaccacaaB |
| RPI Motif I | 18754 | 0.12 | 0.01 | gcacuuGAUₛggcauGcacuaugcgcggcaagcacccB |
| RPI Motif II | SAC | 1.10 | 0.32 | aₛuₛuₛcₛca cUAGuGaggcguuagccGau Acgcga B |
| RPI Motif II | 20339 | 0.85 | 0.01 | uₛcₛcₛuₛcaccUGAuGaggccguuaggccGaaIgggaguB |
| RPI Motif II | 20350 | 1.04 | 0.05 | gₛuₛcₛcₛuggcUGAuGaggccguuaggccGaaIgcugcaB |
| RPI Motif III | Irrelevant Control | 1.28 | 0.07 | ggaaaggugugcaaCCGgaggaaacucCCUUCAAGGACAUCGUCCGGGacggcB |
| RPI Motif III | 18704 | 0.37 | 0.07 | uuccgcagaCGgaggaaacucCCUUCAAGGACGAAAGUCCGGGacuauggB |
| RPI Motif III | 18705 | 0.42 | 0.10 | ccgcagaCGgaggaaacucCCUUCAAGGACGAAAGUCCGGGacuauggB |
| RPI Motif III | 18700 | 0.61 | 0.16 | cagguaguaCGgaggaaacucCCUUCAAGGACAUCGUCCGGGacaaggB |
| RPI Motif III | 18701 | 0.54 | 0.10 | gcacggucUaGgaggaaacucCCUUCAAGGACAUCGUCCGGGgagaccB |
| RPI Motif III | 18835 | 0.54 | 0.04 | guguacucacGgaggaaacucCCUUCAAGGACAUCGUCCGGGgguucB |

Chemical Modifications are indicated as follows:
Lower case = 2′-O-Methyl
Bold (non-italicized): 2′-NH₂
U = 2′-C-Allyl-U
G,A = ribo G,A
s = phosphorothioate linkages
B = inverted abasic
I = ribo Inosine

TABLE X
Anti HCV minus strand Stabilized Ribozyme and Target Sequence

| RPI | | | |
|---|---|---|---|
| No. | Ribozyme Alias | Ribozyme Sequence | Target Sequence |
| 14961 | HCV.5nc-34 Rz-7 allyl stab1 | gₛgₛuₛcₛucg cUGAuGaggccguuaggccGaa Agaccgu B | ACGGUCU A CGAGACC |
| 14962 | HCV.5nc-43 Rz-7 allyl stab1 | gₛcₛcₛcₛcgg cUGAuGaggccguuaggccGaa Aggucuc B | GAGACCU C CCGGGGC |
| 14963 | HCV.5nc-54 Rz-7 allyl stab1 | uₛgₛcₛuₛugc cUGAuGaggccguuaggccGaa Agugccc B | GGGCACU C GCAAGCA |
| 14964 | HCV.5nc-66 Rz-7 allyl stab1 | uₛgₛcₛcₛuga cUGAuGaggccguuaggccGaa Agggugc B | GCACCCU A UCAGGCA |
| 14965 | HCV.5nc-88 Rz-7 allyl stab1 | gₛuₛcₛgₛcga cUGAuGaggccguuaggccGaa Aggccuu B | AAGGCCU U UCGCGAC |
| 14966 | HCV.5nc-88b Rz-7 allyl stab1 | gₛuₛuₛgₛcga cUGAuGaggccguuaggccGaa Aggccuu B | AAGGCCU U UCGCAAC |
| 14967 | HCV.5nc-107 Rz-7 allyl stab1 | gₛcₛuₛaₛgcc cUGAuGaggccguuaggccGaa Aguagug B | CACUACU C GGCUAGC |
| 14968 | HCV.5nc-162 Rz-7 allyl stab1 | uₛuₛuₛcₛuug cUGAuGaggccguuaggccGaa Aucaacc B | GGUUGAU C CAAGAAA |
| 14969 | HCV.5nc-162b Rz-7 allyl stab1 | uₛuₛuₛcₛuug cUGAuGaggccguuaggccGaa Auaaacc B | GGUUUAU C CAAGAAA |
| 14970 | HCV.5nc-192 Rz-7 allyl stab1 | uₛaₛcₛaₛccg cUGAuGaggccguuaggccGaa Aauugcc B | GGCAAUU C CGGUGUA |
| 14971 | HCV.5nc-199 Rz-7 allyl stab1 | cₛgₛgₛuₛgag cUGAuGaggccguuaggccGaa Acaccgg B | CCGGUGU A CUCACCG |
| 14972 | HCV.5nc-202 Rz-7 allyl stab1 | aₛaₛcₛcₛggu cUGAuGaggccguuaggccGaa Aguacac B | GUGUACU C ACCGGUU |
| 14973 | HCV.5nc-222 Rz-7 allyl stab1 | aₛgₛaₛgₛcca cUGAuGaggccguuaggccGaa Agugguc B | GACCACU A UGGCUCU |
| 14974 | HCV.5nc-265 Rz-7 allyl stab1 | uₛuₛaₛgₛuau cUGAuGaggccguuaggccGaa Agugucg B | CGACACU C AUACUAA |
| 14975 | HCV.5nc-33 CHz-7 allyl stab1 | gₛuₛcₛuₛcgu cUGAuGaggccguuaggccGaa Iaccgug B | CACGGUC U ACGAGAC |
| 14976 | HCV.5nc-41 CHz-7 allyl stab1 | cₛcₛcₛgₛgga cUGAuGaggccguuaggccGaa Iucucgu B | ACGAGAC C UCCCGGG |
| 14977 | HCV.5nc-42 CHz-7 allyl stab1 | cₛcₛcₛcₛggg cUGAuGaggccguuaggccGaa Igucucg B | CGAGACC U CCCGGGG |
| 14978 | HCV.5nc-44 CHz-7 allyl stab1 | uₛgₛcₛcₛccg cUGAuGaggccguuaggccGaa Iaggucu B | AGACCUC C CGGGGCA |
| 14979 | HCV.5nc-45 CNz-7 allyl stab1 | gₛuₛgₛcₛccc cUGAuGaggccguuaggccGaa Igagguc B | GACCUCC C GGGGCAC |
| 14980 | HCV.5nc-51 CHz-7 allyl stab1 | uₛuₛgₛcₛgag cUGAuGaggccguuaggccGaa Iccccgg B | CCGGGGC A CUCGCAA |
| 14981 | HCV.5nc-53 CHz-7 allyl stab1 | gₛcₛuₛuₛgcg cUGAuGaggccguuaggccGaa Iugcccc B | GGGGCAC U CGCAAGC |
| 14982 | HCV.5nc-57 CHz-7 allyl stab1 | gₛgₛgₛuₛgcu cUGAuGaggccguuaggccGaa Icgagug B | CACUCGC A AGCACCC |
| 14983 | HCV.5nc-61 CHz-7 allyl stab1 | gₛaₛuₛaₛggg cUGAuGaggccguuaggccGaa Icuugcg B | CGCAAGC A CCCUAUC |
| 14984 | HCV.5nc-63 CHz-7 allyl stab1 | cₛuₛgₛaₛuag cUGAuGaggccguuaggccGaa Iugcuug B | CAAGCAC C CUAUCAG |
| 14985 | HCV.5nc-64 CHz-7 allyl stab1 | cₛcₛuₛgₛaua cUGAuGaggccguuaggccGaa Igugcuu B | AAGCACC C UAUCAGG |
| 14986 | HCV.5nc-65 CHz-7 allyl stab1 | gₛcₛcₛuₛgau cUGAuGaggccguuaggccGaa Iggugcu B | AGCACCC U AUCAGGC |
| 14987 | HCV.5nc-73 CHz-7 allyl stab1 | gₛuₛgₛgₛuac cUGAuGaggccguuaggccGaa Iccugau B | AUCAGGC A GUACCAC |
| 14988 | HCV.5nc-78 CHz-7 allyl stab1 | gₛcₛcₛuₛugu cUGAuGaggccguuaggccGaa Iuacugc B | GCAGUAC C ACAAGGC |
| 14989 | HCV.5nc-79 CHz-7 allyl stab1 | gₛgₛc₂cₛuug cUGAuGaggccguuaggccGaa Iguacug B | CAGUACC A CAAGGCC |
| 14990 | HCV.5nc-81 CHz-7 allyl stab1 | aₛaₛgₛgₛccu cUGAuGaggccguuaggccGaa Iugguac B | GUACCAC A AGGCCUU |
| 14991 | HCV.5nc-87 CHz-7 allyl stab1 | uₛcₛgₛcₛgaa cUGAuGaggccguuaggccGaa Igccuug B | CAAGGCC U UUCGCGA |
| 14992 | HCV.5nc-87b CHz-7 allyl stab1 | uₛuₛgₛcₛgaa cUGAuGaggccguuaggccGaa Igccuug B | CAAGGCC U UUCGCAA |
| 14993 | HCV.5nc-101 CHz-7 allyl stab1 | cₛgₛaₛgₛuag cUGAuGaggccguuaggccGaa Iuugggu B | ACCCAAC A CUACUCG |
| 14994 | HCV.5nc-103 CHz-7 allyl stab1 | gₛcₛcₛgₛagu cUGAuGaggccguuaggccGaa Iuguugg B | CCAACAC U ACUCGGC |
| 14995 | HCV.5nc-106 CHz-7 allyl stab1 | cₛuₛaₛgₛccg cUGAuGaggccguuaggccGaa Iuagugu B | ACACUAC U CGGCUAG |
| 14996 | HCV.5nc-111 CHz-7 allyl stab1 | gₛaₛcₛuₛgcu cUGAuGaggccguuaggccGaa Iccgagu B | ACUCGGC U AGCAGUC |
| 14997 | HCV.5nc-119 CHz-7 allyl stab1 | cₛcₛcₛcₛgcg cUGAuGaggccguuaggccGaa Iacugcu B | AGCAGUC U CGCGGGG |
| 14998 | HCV.5nc-129 CHz-7 allyl stab1 | uₛuₛgₛgₛgcg cUGAuGaggccguuaggccGaa Icccccg B | CGGGGGC A CGCCCAA |
| 14999 | HCV.5nc-163 CHz-7 allyl stab1 | cₛuₛuₛuₛcuu cUGAuGaggccguuaggccGaa Iaucaac B | GUUGAUC C AAGAAAG |
| 15000 | HCV.5nc-163b CHz-7 allyl stab1 | cₛuₛuₛuₛcuu cUGAuGaggccguuaggccGaa Iauaaac B | GUUUAUC C AAGAAAG |
| 15001 | HCV.5nc-164 CHz-7 allyl stab1 | cₛcₛuₛuₛucu cUGAuGaggccguuaggccGaa Igaucaa B | UUGAUCC A AGAAAGG |
| 15002 | HCV.5nc-164b CHz-7 allyl stab1 | cₛcₛuₛuₛucu cUGAuGaggccguuaggccGaa Igauaaa B | UUUAUCC A AGAAAGG |
| 15003 | HCV.5nc-193 CHz-7 allyl stab1 | gₛuₛaₛcₛacc cUGAuGaggccguuaggccGaa Iaauugc B | GCAAUUC C GGUGUAC |
| 15004 | HCV.5nc-201 CHz-7 allyl stab1 | aₛcₛcₛgₛgug cUGAuGaggccguuaggccGaa Iuacacc B | GGUGUAC U CACCGGU |
| 15005 | HCV.5nc-203 CHz-7 allyl stab1 | gₛaₛaₛcₛcgg cUGAuGaggccguuaggccGaa Iaguaca B | UGUACUC A CCGGUUC |
| 15006 | HCV.5nc-205 CHz-7 allyl stab1 | cₛgₛgₛaₛacc cUGAuGaggccguuaggccGaa Iugagua B | UACUCAC C GGUUCCG |
| 15007 | HCV.5nc-211 CHz-7 allyl stab1 | gₛgₛuₛcₛugc cUGAuGaggccguuaggccGaa Iaaccgg B | CCGGUUC C GCAGACC |
| 15008 | HCV.5nc-214 CHz-7 allyl stab1 | aₛgₛuₛgₛguc cUGAuGaggccguuaggccGaa Icggaac B | GUUCCGC A GACCACU |
| 15009 | HCV.5nc-2l8 CHz-7 allyl stab1 | cₛcₛaₛuₛagu cUGAuGaggccguuaggccGaa Iucugcg B | CGCAGAC C ACUAUGG |
| 15010 | HCV.5nc-219 CHz-7 allyl stab1 | gₛcₛcₛaₛuag cUGAuGaggccguuaggccGaa Igucugc B | GCAGACC A CUAUGGC |
| 15011 | HCV.5nc-221 CHz-7 allyl stab1 | gₛaₛgₛcₛcau cUGAuGaggccguuaggccGaa Iuggucu B | AGACCAC U AUGGCUC |
| 15012 | HCV.5nc-227 CHz-7 allyl stab1 | cₛcₛgₛgₛgag cUGAuGaggccguuaggccGaa Iccauag B | CUAUGGC U CUCCCGG |
| 15013 | HCV.5nc-229 CHz-7 allyl stab1 | uₛcₛcₛcₛggg cUGAuGaggccguuaggccGaa Iagccau B | AUGGCUC U CCCGGGA |
| 15014 | HCV.5nc-231 CHz-7 allyl stab1 | cₛcₛuₛcₛccg cUGAuGaggccguuaggccGaa Iagagcc B | GGCUCUC C CGGGAGG |
| 15015 | HCV.5nc-232 CHz-7 allyl stab1 | cₛcₛcₛuₛccc cUGAuGaggccguuaggccGaa Igagagc B | GCUCUCC C GGGAGGG |
| 15016 | HCV.5nc-266 CHz-7 allyl stab1 | gₛuₛuₛaₛgua cUGAuGaggccguuaggccGaa Iaguguc B | GACACUC A UACUAAC |
| 15017 | HCV.5nc-270 CHz-7 allyl stab1 | uₛgₛgₛcₛguu cUGAuGaggccguuaggccGaa Iuaugag B | CUCAUAC U AACGCCA |
| 15018 | HCV.5-31 CHz-7 allyl stab1 | uₛcₛaₛcₛagg cUGAuGaggccguuaggccGaa Iagugau B | AUCACUC C CCUGUGA |
| 15019 | HCV.5-32 CHz-7 allyl stab1 | cₛuₛcₛaₛcag cUGAuGaggccguuaggccGaa Igaguga B | UCACUCC C CUGUGAG |
| 15020 | HCV.5-33 CHz-7 allyl stab1 | cₛcₛuₛcₛaca cUGAuGaggccguuaggccGaa Iggagug B | CACUCCC C UGUGAGG |
| 15021 | HCV.5-34 CHz-7 allyl stab1 | uₛcₛcₛuₛcac cUGAuGaggccguuaggccGaa Igggagu B | ACUCCCC U GUGAGGA |
| 15022 | HCV.5-44 CHz-7 allyl stab1 | aₛgₛaₛcₛagu cUGAuGaggccguuaggccGaa Iuuccuc B | GAGGAAC U ACUGUCU |
| 15023 | HCV.5-47 CHz-7 allyl stab1 | uₛgₛaₛaₛgac cUGAuGaggccguuaggccGaa Iuaguuc B | GAACUAC U GUCUUCA |
| 15024 | HCV.5-51 CHz-7 allyl stab1 | uₛgₛcₛgₛuga cUGAuGaggccguuaggccGaa Iacagua B | UACUGUC U UCACGCA |
| 15025 | HCV.5-54 CHz-7 allyl stab1 | uₛuₛcₛuₛgcg cUGAuGaggccguuaggccGaa Iaagaca B | UGUCUUC A CGCAGAA |
| 15026 | HCV.5-58 CHz-7 allyl stab1 | cₛgₛcₛuₛuuc cUGAuGaggccguuaggccGaa Icgugaa B | UUCACGC A GAAAGCG |
| 15027 | HCV.5-68 CHz-7 allyl stab1 | cₛaₛuₛgₛgcu cUGAuGaggccguuaggccGaa Iacgcuu B | AAGCGUC U AGCCAUG |
| 15028 | HCV.5-72 CHz-7 allyl stab1 | aₛcₛgₛcₛcau cUGAuGaggccguuaggccGaa Icuagac B | GUCUAGC C AUGGCGU |
| 15029 | HCV.5-73 CHz-7 allyl stab1 | aₛaₛcₛgₛcca cUGAuGaggccguuaggccGaa Igcuaga B | UCUAGCC A UGGCGUU |
| 15030 | HCV.5-97 CHz-7 allyl stab1 | uₛgₛgₛaₛggc cUGAuGaggccguuaggccGaa Icacgac B | GUCGUGC A GCCUCCA |
| 15031 | HCV.5-100 CHz-7 allyl stab1 | uₛcₛcₛuₛgga cUGAuGaggccguuaggccGaa Icugcac B | GUGCAGC C UCCAGGA |
| 15032 | HCV.5-101 CHz-7 allyl stab1 | gₛuₛcₛcₛugg cUGAuGaggccguuaggccGaa Igcugca B | UGCAGCC U CCAGGAC |
| 15033 | HCV.5-103 CHz-7 allyl stab1 | gₛgₛgₛuₛccu cUGAuGaggccguuaggccGaa Iaggcug B | CAGCCUC C AGGACCC |
| 15034 | HCV.5-104 CHz-7 allyl stab1 | gₛgₛgₛgₛucc cUGAuGaggccguuaggccGaa Igaggcu B | AGCCUCC A GGACCCC |
| 15035 | HCV.5-109 CHz-7 allyl stab1 | gₛaₛgₛgₛggg cUGAuGaggccguuaggccGaa Iuccugg B | CCAGGAC C CCCCCUC |
| 15036 | HCV.5-llO CHz-7 allyl stab1 | gₛgₛaₛgₛggg cUGAuGaggccguuaggccGaa Iguccug B | CAGGACC C CCCCUCC |
| 15037 | HCV.5-ll1 CHz-7 allyl stab1 | gₛgₛgₛaₛggg cUGAuGaggccguuaggccGaa Igguccu B | AGGACCC C CCCUCCC |
| 15038 | HCV.5-112 CHz-7 allyl stab1 | cₛgₛgₛgₛagg cUGAuGaggccguuaggccGaa Igggucc B | GGACCCC C CCUCCCG |
| 15039 | HCV.5-113 CHz-7 allyl stab1 | cₛcₛgₛgₛgag cUGAuGaggccguuaggccGaa Igggguc B | GACCCCC C CUCCCGG |
| 15040 | HCV.5-114 CHz-7 allyl stab1 | cₛcₛcₛgₛgga cUGAuGaggccguuaggccGaa Igggggu B | ACCCCCC C UCCCGGG |
| 15041 | HCV.5-115 CHz-7 allyl stab1 | uₛcₛcₛcₛggg cUGAuGaggccguuaggccGaa Igggggg B | CCCCCCC U CCCGGGA |
| 15042 | HCV.5-117 CHz-7 allyl stab1 | uₛcₛuₛcₛccg cUGAuGaggccguuaggccGaa Iaggggg B | CCCCCUC C CGGGAGA |
| 15043 | HCV.5-118 CHz-7 allyl stab1 | cₛuₛcₛuₛccc cUGAuGaggccguuaggccGaa Igagggg B | CCCCUCC C GGGAGAG |
| 15044 | HCV.5-127 CHz-7 allyl stab1 | cₛcₛaₛcₛuau cUGAuGaggccguuaggccGaa Icucucc B | GGAGAGC C AUAGUGG |
| 15045 | HCV.5-128 CHz-7 allyl stab1 | aₛcₛcₛaₛcua cUGAuGaggccguuaggccGaa Igcucuc B | GAGAGCC A UAGUGGU |
| 15046 | HCV.5-137 CHz-7 allyl stab1 | gₛuₛuₛcₛcgc cUGAuGaggccguuaggccGaa Iaccacu B | AGUGGUC U GCGGAAC |
| 15047 | HCV.5-145 CHz-7 allyl stab1 | aₛcₛuₛcₛacc cUGAuGaggccguuaggccGaa Iuuccgc B | GCGGAAC C GGUGAGU |
| 15048 | HCV.5-155 CHz-7 allyl stab1 | aₛuₛuₛcₛcgg cUGAuGaggccguuaggccGaa Iuacuca B | UGAGUAC A CCGGAAU |
| 15049 | HCV.5-157 CHz-7 allyl stab1 | cₛaₛaₛuₛucc cUGAuGaggccguuaggccGaa Iuguacu B | AGUACAC C GGAAUUG |
| 15050 | HCV.5-181 CHz-7 allyl stab1 | cₛaₛaₛgₛaaa cUGAuGaggccguuaggccGaa Iacccgg B | CCGGGUC C UUUCUUG |
| 15051 | HCV.5-182 CHz-7 allyl stab1 | cₛcₛaₛaₛgaa cUGAuGaggccguuaggccGaa Igacccg B | CGGGUCC U UUCUUGG |
| 15052 | HCV.5-186 CHz-7 allyl stab1 | uₛgₛaₛuₛcca cUGAuGaggccguuaggccGaa Iaaagga B | UCCUUUC U UGGAUCA |

What we claim is:

1. An enzymatic nucleic acid molecule which specifically cleaves minus strand RNA derived from hepatitis C virus (HCV), wherein the binding arms of said enzymatic nucleic acid molecule comprises sequences complementary to any of substrate sequences defined in Table X.

2. An enzymatic nucleic acid molecule which specifically cleaves minus strand RNA derived from hepatitis C virus (HCV), wherein said enzymatic nucleic acid molecule comprises sequences defined as ribozyme sequences in Table X.

3. An enzymatic nucleic acid molecule which selectively cleaves RNA derived from HCV, wherein said enzymatic nucleic acid molecule is selected from the group consisting of inozyme, G-cleaver, DNAzyme, Amberzyme, and Zinzyme motifs.

4. The enzymatic nucleic acid molecule of claim 3, wherein said enzymatic nucleic acid molecule cleaves plus strand RNA derived from HCV.

5. The enzymatic nucleic acid molecule of claim 3, wherein said enzymatic nucleic acid molecule cleaves minus strand RNA derived from HCV.

6. The enzymatic nucleic acid molecule of claim 3, wherein said inozyme enzymatic nucleic acid molecule comprises a stem II region of length greater than or equal to 2 base pairs.

7. The enzymatic nucleic acid molecule of claim 1, wherein said enzymatic nucleic acid molecule is selected from the group consisting of hammerhead (HH), G-cleaver, Inozyme, DNAzyme, Amberzyme, and Zinzyme motifs.

8. The enzymatic nucleic acid molecule of any of claims 1 and 3, wherein said enzymatic nucleic acid comprises between 12 and 100 bases complementary to said RNA derived from HCV.

9. The enzymatic nucleic acid molecule of any of claims 1 and 3, wherein said enzymatic nucleic acid comprises between 14 and 24 bases complementary to said RNA derived from HCV.

10. A pharmaceutical composition comprising the enzymatic nucleic acid molecule of any of claims 1 and 3.

11. A mammalian cell including an enzymatic nucleic acid molecule of any of claims 1 and 3.

12. The mammalian cell of claim 11, wherein said mammalian cell is a human cell.

13. An expression vector comprising nucleic acid sequence encoding at least one enzymatic nucleic acid molecule of claims 1 or 3, in a manner which allows expression of that enzymatic nucleic acid molecule.

14. A mammalian cell including an expression vector of claim 13.

15. The mammalian cell of claim 14, wherein said mammalian cell is a human cell.

16. A method for treatment of cirrhosis, liver failure or hepatocellular carcinoma comprising the step of administering to a patient the enzymatic nucleic acid molecule of any of claims 1 and 3 under conditions suitable for said treatment.

17. A method for treatment of cirrhosis, liver failure and/or hepatocellular carcinoma comprising the step of administering to a patient the expression vector of claim 13 under conditions suitable for said treatment.

18. A method of treatment of a patient having a condition associated with HCV infection, comprising contacting cells of said patient with the nucleic acid molecule of any of claims 1 and 3, and further comprising the use of one or more drug therapies under conditions suitable for said treatment.

19. A method for inhibiting HCV replication in a mammalian cell comprising the step of administering to said cell the enzymatic nucleic acid molecule of any of claims 1 and 3 under conditions suitable for said inhibition.

20. A method of cleaving a separate RNA molecule comprising, contacting the enzymatic nucleic acid molecule of any of claims 1 and 3 with said separate RNA molecule under conditions suitable for the cleavage of said separate RNA molecule.

21. The method of claim 20, wherein said cleavage is carried out in the presence of a divalent cation.

22. The method of claim 21, wherein said divalent cation is Mg²⁺.

23. The nucleic acid molecule of claims 1 or 3, wherein said nucleic acid is chemically synthesized.

24. The expression vector of claim 13, wherein said vector comprises:
a. a transcription initiation region;
b. a transcription termination region;
c. a nucleic acid sequence encoding at least one said nucleic acid molecule; and
wherein said sequence is operably linked to said initiation region and said termination region, in a manner which allows expression and/or delivery of said nucleic acid molecule.

25. The expression vector of claim 13, wherein said vector comprises:
a. a transcription initiation region;
b. a transcription termination region;
c. an open reading frame;
d. a nucleic acid sequence encoding at least one said nucleic acid molecule, wherein said sequence is operably linked to the 3′-end of said open reading frame; and
wherein said sequence is operably linked to said initiation region, said open reading frame and said termination region, in a manner which allows expression and/or delivery of said nucleic acid molecule.

26. The expression vector of claim 13, wherein said vector comprises:
a. a transcription initiation region;
b. a transcription termination region;
c. an intron;
d. a nucleic acid sequence encoding at least one said nucleic acid molecule; and
wherein said sequence is operably linked to said initiation region, said intron and said termination region, in a manner which allows expression and/or delivery of said nucleic acid molecule.

27. The expression vector of claim 13, wherein said vector comprises:
a. a transcription initiation region;
b. a transcription termination region;
c. an intron;
d. an open reading frame;
e. a nucleic acid sequence encoding at least one said nucleic acid molecule, wherein said sequence is operably linked to the 3′-end of said open reading frame; and
wherein said sequence is operably linked to said initiation region, said intron, said open reading frame and said termination region, in a manner which allows expression and/or delivery of said nucleic acid molecule.

28. The enzymatic nucleic acid molecule of claims 1 or 3, wherein said enzymatic nucleic acid comprises at least one 2′-sugar modification.

29. The enzymatic nucleic acid molecule of claims 1 or 3, wherein said enzymatic nucleic acid comprises at least one nucleic acid base modification.

30. The enzymatic nucleic acid molecule of claims 1 or 3, wherein said enzymatic nucleic acid comprises at least one phosphate modification.

31. The method of claim 18, wherein said drug therapies is type I interferon.

32. The method of claim 31, wherein said type I interferon and the enzymatic nucleic acid molecule are administered simultaneously.

33. The method of claim 31, wherein said type I interferon and enzymatic nucleic acid molecule are administered separately.

34. The method of claim 31, wherein said type I interferon is interferon alpha.

35. The method of claim 31, wherein said type I interferon is interferon beta.

36. The method of claim 31, wherein said type I interferon is interferon gamma.

37. The method of claim 31, wherein said type I interferon is consensus interferon.
